(19) United States
(12) Patent Application Publication
(10) Pub. No.: US 2025/0290128 A1
(43) Pub. Date: Sep. 18, 2025

(54) METHOD OF MEASURING MICROSATELLITE LENGTH VARIATIONS

(71) Applicant: Cold Spring Harbor Laboratory, Cold Spring Harbor, NY (US)

(72) Inventors: Michael Wigler, Cold Spring Harbor, NY (US); Dan Levy, Cold Spring Harbor, NY (US); Siran Li, Cold Spring Harbor, NY (US); Zihua Wang, Cold Spring Harbor, NY (US); Andrea Moffitt, Cold Spring Harbor, NY (US); Peter Andrews, Cold Spring Harbor, NY (US)

(73) Assignee: Cold Spring Harbor Laboratory, Cold Spring Harbor, NY (US)

(21) Appl. No.: 18/690,018

(22) PCT Filed: Sep. 9, 2022
(86) PCT No.: PCT/US2022/076178

§ 371 Date: Mar. 7, 2024

Related U.S. Application Data
(60) Provisional application No. 63/243,033, filed on Sep. 10, 2021.
(60) Provisional application No. 63/263,479, filed on Nov. 3, 2021.
(60) Provisional application No. 63/263,716, filed on Nov. 8, 2021.

(51) Int. Cl. C12Q 1/6869; C12N 15/10; C12Q 1/37; C12Q 1/6806; C12Q 1/686

(52) U.S. Cl. CPC C12Q 1/6869; C12N 15/1058; C12Q 1/37; C12Q 1/6806; C12Q 1/686; C12Q 2600/156

(57) ABSTRACT

This invention provides a method for obtaining microsatellite lengths from initial nucleic acid templates each of which comprise a microsatellite and two flanking portions, which define the microsatellite and its locus.

### CROSS-REFERENCE TO RELATED APPLICATIONS

This application is a § 371 national stage of PCT International Application No. PCT/US2022/076178, filed Sep. 9, 2022, claiming the benefit of U.S. Provisional Application No. 63/263,716, filed Nov. 8, 2021, U.S. Provisional Application No. 63/263,479 filed Nov. 3, 2021, and U.S. Provisional Application No. 63/243,033 filed Sep. 10, 2021, the contents of each of which are hereby incorporated by reference into the subject application.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “241028_91762-A-PCT-US_Sequence_Listing_DH.xml”, which is 3,433,049 bytes in size, and which was created on Oct. 25, 2024 in the IBM-PC machine format, having an operating system compatibility with MS-Windows, which is contained in the xml file filed Oct. 28, 2024 as part of this application.

### BACKGROUND OF THE INVENTION

Replication of tandem repeats of simple sequence motifs, also known as microsatellites, is error prone and variable lengths frequently occur during population expansions. Therefore, microsatellite length variations could serve as markers for cancer. However, accurate error-free quantitation of microsatellite lengths is difficult with current methods because of a high error rate during amplification and sequencing.

Tumors have genomic variants that distinguish them from the germline genome. These include single nucleotide variants (SNVs), small indels, large scale copy number variation (CNVs), and microsatellite length variation (MSLV). The profile of tumor variation has value in outcome prediction, the measurement of minimal residual disease, and possibly the early detection of cancer.

Accurate measurement of MSLV remains a challenge in the art. This class of variation is very abundant in cancers from patients with defects in mismatch repair (Eshleman, J. R. & Markowitz, S. D., 1996; Lynch, H. T. et al., 2009), but also in cancers in general (Bonneville, R. et al. 2017; Fujimoto, A. et al., 2020; Hause, R. J., et al., 2016; Kim, T. M., et al., 2013). If MS lengths could be typed accurately it would open a potentially efficient way to fingerprint a cancer and to measure its presence in clinical specimens such as tissue biopsies, blood and urine (Georgiadis, A. et al., 2019; Silveira, A. B. et al., 2020). The problem is that the same property of microsatellites that make their length unstable in cancer (and causes extensive heterogeneity in germline populations), namely, the repeat of a simple sequence motif, makes them unstable during amplification and sequencing. The microsatellite can expand or contract by units of the repeat, presumably due to polymerase slippage during replication (Clarke, L. A., et al., 2001, Kunkel, T. A., 1986; Lai, Y., et al., 2003; Shinde, D., et al. 2003). This is particularly problematic when measuring the lengths of mononucleotide repeats, which are the most highly variable type of repeat in cancers (Bonneville, R. et al. 2017; Fujimoto, A. et al., 2020; Hause, R. J., et al., 2016; Kim, T. M., et al., 2013). In addition, modern day high throughput sequence platforms fail to read mono-nucleotide tracts accurately (Stoler, N. & Nekrutenko, A., 2021; Zavodna, M., et al., 2014).

To tackle this problem, various approaches have been applied to date. Multiplex PCR and capillary electrophoresis has been described with 5-10 microsatellite loci (Bacher, J. W. et al., 2004; Boland, C. R. et al., 1998; Murphy, K. M. et al., 2006). MS lengths have been characterized from gene panels and high throughput sequencing (Georgiadis, A. et al., 2019; Middha, S. et al., 2017), and statistical methods have been developed to increase accuracy in calling MS lengths (Fungtammasan, A. et al., 2015; Highnam, G. et al., 2013). Finally, droplet digital PCR has also been employed (Silveira, A. B. et al., 2020) to increase accuracy for small numbers of loci. None of these methods has the scale, depth, generality and accuracy all of which would be required for routinely monitoring a large panel of microsatellite loci.

### SUMMARY OF THE INVENTION

This invention provides a method for measuring microsatellite lengths of initial nucleic acid templates each of which comprise a microsatellite and two flanking portions, which flanking portions delineate the microsatellite, the method comprising:
(a) generating partially mutagenized templates by:
   (i) partial mutagenesis of the initial nucleic acid templates;
   (ii) partial mutagenesis during production of first copies of the initial nucleic acid templates; and/or
   (iii) generating first copies of the initial nucleic acid templates followed by partial mutagenesis of the first copies of the initial nucleic acid templates;
(b) making a sequencing library from the partially mutagenized templates;
(c) sequencing the library from step (b) to generate sequence reads;
(d) selecting a set of sequence reads in which the microsatellite has been disrupted by mutagenesis so that
   (i) at least two, preferably more of the tandem repeat units in the microsatellite have been mutagenized; and
   (ii) the microsatellite in the sequence read has no more than 7, preferably 5 or fewer, tandem repeats in a row; and
(e) measuring microsatellite lengths from the set of sequence reads selected in step
(d), wherein the measurement is the distance between the flanking portions that delineate the microsatellite of the initial templates.

This invention also provides a method for measuring microsatellite lengths of initial nucleic acid templates each of which comprise a microsatellite and two flanking portions, which flanking portions delineate the microsatellite, the method comprising:
(a) generating partially mutagenized templates by:
   (i) partial mutagenesis of the initial nucleic acid templates;
   (ii) partial mutagenesis during production of first copies of the initial nucleic acid templates; and/or
   (iii) generating first copies of the initial nucleic acid templates followed by partial mutagenesis of the first copies of the initial nucleic acid templates; making a sequencing library from the partially mutagenized templates;
(c) sequencing the library from step (b) to generate sequence reads;
(d) selecting a set of sequence reads in which the microsatellite exceeds a disruption index so that the error rate between matched pairs is less than 2%, less than 1%, or preferably less than 0.5%, wherein matched pairs are independent reads that share a template, first copy, or locus; and
(e) measuring microsatellite lengths from the set of sequence reads selected in step (d), wherein the measurement is the distance between the flanking portions that delineate the microsatellite of the initial templates.

This invention also provides a sequencing library comprising nucleic acid templates, wherein at least 5%, preferably at least 10%, more preferably at least 20%, and more preferably at least 30% of the nucleic acid templates comprise:
(a) a mutagenized microsatellite in which:
   (i) at least two, preferably more of the tandem repeat units in the microsatellite have been mutagenized; and
   (ii) the mutagenized microsatellite has no more than 7, preferably 5 or fewer, tandem repeats in a row; and
(b) two flanking portions.

This invention also provides a sequencing library comprising nucleic acid templates which comprise:
(a) a mutagenized microsatellite in which:
   (i) at least two, preferably more of the tandem repeat units in the microsatellite have been mutagenized; and
   (ii) the mutagenized microsatellite has no more than 7, preferably 5 or fewer, tandem repeats in a row; and
(b) flanking portions which are not mutagenized.

### BRIEF DESCRIPTION OF THE DRAWINGS

FIG. 1: Template sequence with microsatellite. Top: An example of a template sequence is shown, with a C microsatellite (MS) of length 17, from position 25 to 41 in the sequence, of length 17. The left flank sequence (LF) ends with A at position 24, the left boundary. The right flank sequence (RF) starts with A at position 42, the right boundary. The MS length is delineated by the left and right boundaries, and is of length 43−24+1=17. Middle: For reads that derive from the above template after partial mutagenesis, specifically bisulfite treatment, some portion of the C's in the read will be read as T's, both within the microsatellite and within the flanks. C's that are read as T's are indicated in bold. Bottom: To process the mutated read computationally, all C's are converted to T's. These bases are shown in bold.

FIG. 2: Searching for flank sequences. Top: To search for flank sequences in the fully converted reads, the search string for the flank sequences are computationally converted as well, shown by C to T bases in bold. Bottom: Fully converted flank sequences are aligned with a fully converted read sequence to define the left and right boundaries of the microsatellite. Single nucleotide polymorphisms (SNPs) or single base sequencing errors (bold italic base) in the read can be tolerated in the flank-read match.

FIG. 3: Bench protocol for partial mutagenesis. In step 1, each of the two synthetic templates containing C were partially bisulfite converted. In steps 2-3, about 6*10\hat{ }4 of these templates underwent 9 cycles of linear amplification by using a biotinylated oligo. In step 4, double-stranded DNA fragments were obtained in another round of linear amplification by using UP1. In step 5, extra free oligo was removed by exonuclease I. After adding carrier DNA, biotinylated DNA fragments were purified by streptavidin beads. In step 6, the exponential PCR was carried out using UP1 and UP3 to generate enough material to prepare the DNA libraries for sequencing. These were sequenced as 2×150 bp paired-end runs on MiSeq (steps 7-8).

FIG. 4: Reads per first copies. For each of the 5 libraries, the reads per first copy distributions used to establish a cutoff for minimum reads for a first copy to be well-covered are shown. Each first copy has a point on the graph, with the x-axis indicating its position in a sorted list by number of reads, and the y-axis indicating the number of reads assigned to that first copy.

FIG. 5: Luria-Delbruck Diffusion Code. Python code for simulating Luria-Delbruck Diffusion is shown.

FIG. 6A to FIG. 6F: Sequencing base quality and base composition. For each of the 5 libraries, the raw sequencing reads are split into 4 sets, based on read number 1 or 2, and base composition of the microsatellite (A vs. T, C vs. G, CA vs. TG). For each set of reads, the per base Phred-scale quality score is shown, as well as the per base composition of A/C/G/T. In the base quality plot, the black dashed line indicates mean base quality; the box plot shows the 25^{th} and 75^{th} quantiles in grey shading and the 10^{th} and 90^{th} quantiles in black error bars; the grey horizontal line appearing within the grey shaded box plot shows median base quality. The microsatellite region is highlighted with shading in both plots. Base quality drops dramatically after reading through the mononucleotide microsatellites. This impact on base quality is not observed in the mutated libraries.

FIG. 7: Drop-out and matching tract lengths. For each of the unmutated mononucleotide tracts, M-17 (A−) and M-18 (C−), a scatterplot of the measured lengths from the two reads in the pair is shown, with the length measured from the read reading the mono tract as A or C track on the x-axis and the length measured from the read reading the mono tract as a T or G track on the y-axis. The −1 position is reserved for those cases where both strands were unable to make a microsatellite call. This plot contains a down-sampling to 1 million data points.

FIG. 8: MSL distributions for reads and first copies. For each of the five libraries, distribution of observed MSLV is plotted. The upper panels show the distribution of read counts and the lower panels show the distribution of first copy modal lengths. The expected length is shown in darker grey, with off-target lengths shown in lighter grey. The plot legends summarize the on and off-target rates per library. Data used to generate these plots is included in Table 1.

FIG. 9: First copy consensus per template. For three libraries, the distribution of templates with x on-target first copies and y off-target first copies is plotted. The size of the dot and darkness of the shading reflect the proportion of templates, normalized by the total template count. Templates with no on-target first copies were further divided into “U” if all the first copies were unanimous for the same off-target length, or “M” if the first copies lengths were mixed. To highlight the population, unanimous off-target template populations are shown in the circles with x's through them (®).

FIG. 10: Mutagenesis library overview. Stages of a partial mutagenesis protocol are demonstrated for initial templates with different degrees of mutagenesis and disruption. Lighter grey arrows in the template indicate unites of a microsatellite repeat. The darker grey arrows in the template indicate mutation in that repeat unit. Length of the microsatellite sequence is indicated in the circle above the repeat. The original length of the initial template is L; increases in length are indicated by +1, +2, etc.; decreases in length are indicated by −1, −2, etc. Tags for the templates are #1, #2, etc. and tags for each first copy are #1A, #1B, . . . , #1N, etc. Stages 1˜4 are part of the sequencing library preparation. Stages 6, 7, and 8 illustrate results from data analysis. The distributions of lengths observed are plotted for reads, first copies, and templates, with on-target length L shaded lighter grey and off-target lengths shaded darker grey.

FIG. 11: Protocol for enriched sequencing library. The diagram shows a protocol for making sequencing libraries with partial bisulfite mutagenesis and hybridization capture enrichment of microsatellite loci from genomic DNA. In step 1, double stranded molecules are processed with end-polishing and A-tailing. In step 2, fish-tail adapters with varietal tags, sample barcodes, and universal primers are ligated to the double-stranded molecules. Sequences shown as empty boxes indicate no C's in the sequence. Step 3 is the hybridization capture panel based enrichment with the C containing strand as the target of this enrichment. Step 4 is partial bisulfite mutagenesis. Step 5 is multiple cycles of linear amplification using a universal primer and additional varietal tag. Step 6 is one cycle of linear amplification with a biotinylated oligo. Step 7 is purification of the biotinylated oligos and exponential amplification with the p5 and p7 primers to generate a DNA sequencing library. Libraries are then sequenced to generate sequencing read data.

FIG. 12: Coverage across panel loci. The number of reads mapped to each of 630 panel loci is shown as a ranked plot, with the locus with the most mapped reads on the right and the locus with the least mapped reads on the left. Coverage is relatively uniform for both the C panel (top) and AC panel (bottom) indicating even enrichment across the panel.

FIG. 13: Degrees of disruption. Examples of different degrees of disruption is shown for the same sequence, with 8 different levels of mutagenesis. The microsatellite sequence in the middle of the sequence is indicated with and C's that have been converted to T's via mutagenesis are bolded. The longest remaining repeat sequence is indicated with the horizontal bracket. The table on the right shows the overall conversion rate (percentage of C's in the full sequence converted to T); the MS conversion rate (percentage of C's in the 20 base microsatellite sequence converted to T); and the max repeat length (largest number of consecutive C's or T's in the sequence). No mutagenesis leads to a 0% overall and MS conversion rate, while full mutagenesis leads to a 100% overall and MS conversion rate, both with 20 base max repeat lengths. Partial mutagenesis may lead to a MS that is not disrupted, mildly disrupted, or highly disrupted, depending on the MS conversion rate and max repeat length remaining after mutagenesis.

FIG. 14: Distinguishing mixtures of populations with different microsatellite lengths. The diagram shows a conceptual example of microsatellite length distributions from different types of samples, with and without disrupting the microsatellite sequence. With disruption, the observed length distribution is a highly accurate representation of the true distribution. Without disruption, the observed distribution is a highly distorted representation of the true distribution. The first two rows show distributions from samples with only one microsatellite length (at a single locus), such as pure germline (dark grey) or pure tumor (black with white dots). The lower three rows show observed distributions when the tumor and germline are mixed (light grey), as is expected in a tumor biopsy, or in cell-free DNA. The distortion without disruption results in data that cannot reliably distinguish the presence of a rare microsatellite length from the tumor present in 1% of cell-free DNA from a case where no tumor DNA is present in the cell-free DNA (bottom two rows).

FIG. 15: Determining and detecting the tumor microsatellite signature in a tumor biopsy and cell-free DNA. The diagram shows a conceptual example of characterizing microsatellite lengths in a tumor biopsy and in cell-free DNA. The top panel shows the comparison of the MS length distributions in a tumor biopsy and a blood cell sample to determine that the length 17 (black with white dots) is associated with the tumor. The middle panel extends this comparison to many loci, identifying the tumor-associated length in black with white dots and determining which loci have a unique tumor-associated length that will be useful for distinguishing tumor from normal in a mixture. The bottom panel shows the frequencies of MS length variants that might be expected when examining those same loci in cell-free DNA before and after treatment.

FIG. 16: Early detection of a neoplasm from circulating cell-free DNA. The diagram shows a conceptual example of early detection of a neoplasm by monitoring microsatellite lengths in circulating cell-free DNA. For N loci (columns) MS length distributions are shown for the putative germline, where each locus has 1 or 2 lengths present (top panel). In the lower panels, the distributions are shown for the same loci in a baseline, +1 year, and +2 year samples. Dark grey indicates the majority germline length; black with white dots indicates a lower frequency somatic variation that is detected in both blood cells and the circulating cell-free DNA; light grey with black dots indicates a somatic variation that is not in the baseline and grows in frequency over time. The light grey with black dots length is not detected in the blood cells, indicating that it may arise from cells in a non-blood tissue. Its rapid expansion and presence only in cell-free DNA may indicate a rapid clonal expansion somewhere in the individual and may suggest more intensive cancer screenings for the individual at that time.

FIG. 17: Partial disruption lowers error rate of MSL in panel data, exactly as predicted. Using panels, microsatellites from loci were enriched, 630 with mononucleotide C repeats (top panel) and 630 with dinucleotide AC repeats (bottom panel), each of varying lengths (see X-axis). These enriched fragments were partially mutagenized with bisulfite, or not. Sequencing libraries were separately prepared from the mutagenized and unmutagenized fragments (top line in each graph). Following sequencing, the reads from the mutagenized library were scored as mutated but insufficiently disrupted (middle line in each graph) or sufficiently disrupted as described in the current application (bottom line in each graph). The error rate (Y-axis, log scale) is the proportion of PCR duplicates, defined by template tag and mapping, disagreeing for the MS length. Disagreements are assigned to the longer of the two lengths. 95% confidence intervals for the error rates are determined from the total number of observations using Jefferys interval, with intervals including zero truncated to 10⁻⁶. The expected error rate, based on the data for synthetic templates C-18 and AC-26, are indicated with the star shaded to match the corresponding case.

FIG. 18: Demonstration that directed partial bisulfite mutagenesis works. The figure shows every cytosine in the synthetic template: its position in the template displayed on the X-axis, and the proportion of conversion from C to T on the Y-axis. Panel A shows the proportion of conversion from C to T with templates alone, while Panels B and C show the proportion of conversion from C to T when blockers are used. In Panel B the reaction conditions were: 55 degrees centigrade for 30 minutes. In Panel C the reaction conditions were: 40 degrees for 70 minutes. The vertical lines denote the boundaries of the microsatellite.

FIG. 19: A plot of tumor vs normal eccentricity. For both high-MSI patients (53 and 55) the eccentricity in the tumor exceeded the blood in ˜94% of mono-C loci and ˜62% of di-AC loci. In contrast, in a low MSI patient (61), the tumor eccentricity exceeded the blood 51% of the time (practically random) and for the di-AC loci, the tumor eccentricity exceeded the blood 38% of the time.

FIG. 20: The distribution of mean eccentricity values for the three patients and the accompanying power curves computed using pure blood data as the null distribution.

### DETAILED DESCRIPTION OF THE INVENTION

This invention provides a method for measuring microsatellite lengths of initial nucleic acid templates each of which comprise a microsatellite and two flanking portions, which flanking portions delineate the microsatellite, the method comprising:
(a) generating partially mutagenized templates by:
   (i) partial mutagenesis of the initial nucleic acid templates;
   (ii) partial mutagenesis during production of first copies of the initial nucleic acid templates; and/or
   (iii) generating first copies of the initial nucleic acid templates followed by partial mutagenesis of the first copies of the initial nucleic acid templates;
(b) making a sequencing library from the partially mutagenized templates;
(c) sequencing the library from step (b) to generate sequence reads;
(d) selecting a set of sequence reads in which the microsatellite has been disrupted by mutagenesis so that
   (i) at least two, preferably more of the tandem repeat units in the microsatellite have been mutagenized; and
   (ii) the microsatellite in the sequence read has no more than 7, preferably 5 or fewer, tandem repeats in a row; and
(e) measuring microsatellite lengths from the set of sequence reads selected in step (d), wherein the measurement is the distance between the flanking portions that delineate the microsatellite of the initial templates.

This invention also provides a method for obtaining a distribution of microsatellite lengths from initial nucleic acid templates each of which comprise a microsatellite and two flanking portions, which define the microsatellite and its locus, the method comprising:
(a) generating partially mutagenized copies of the initial nucleic acid templates by:
   (i) partial mutagenesis of the initial nucleic acid templates;
   (ii) producing partially mutagenized first copies of the initial nucleic acid templates;
   (iii) producing first copies of the initial nucleic acid templates followed by partial mutagenesis of the first copies of the initial nucleic acid templates;
(b) sequencing the partially mutagenized copies of the initial nucleic acid templates to generate sequence reads,
(c) selecting a set of sequence reads in which the microsatellite has been disrupted by mutagenesis so that:
   (i) at least two, preferably more of the tandem repeat units in the microsatellite have been mutagenized;
   (ii) the microsatellite in the sequence read has no more than 7, preferably 5 or fewer, tandem repeats in a row; and
(d) measuring microsatellite lengths from the set of sequence reads selected in step (c) as the distance between the flanking portions that delineate the microsatellite of the initial templates, wherein a distribution of measured microsatellite lengths from the selected set of sequence reads corresponding to an individual locus is obtained.

In embodiments, as an alternative to the step of “selecting a set of sequence reads in which the microsatellite has been disrupted by mutagenesis so that (i) at least two, preferably more of the tandem repeat units in the microsatellite have been mutagenized, and (ii) the microsatellite in the sequence read has no more than 7, preferably 5 or fewer, tandem repeats in a row” in the methods described herein, the step instead comprises selecting a set of sequence reads in which the microsatellite exceeds a disruption index so that the error rate between matched pairs is less than 2%, or 1%, or preferably less than 0.5%; wherein matched pairs are independent reads that share a template, first copy, or locus. In this context, “independent reads” means two reads that do not come from the same sequencing library element.

Accordingly, this invention provides a method for measuring microsatellite lengths of initial nucleic acid templates each of which comprise a microsatellite and two flanking portions, which flanking portions delineate the microsatellite, the method comprising:
(a) generating partially mutagenized templates by:
   (i) partial mutagenesis of the initial nucleic acid templates;
   (ii) partial mutagenesis during production of first copies of the initial nucleic acid templates; and/or
   (iii) generating first copies of the initial nucleic acid templates followed by partial mutagenesis of the first copies of the initial nucleic acid templates; making a sequencing library from the partially mutagenized templates;
(c) sequencing the library from step (b) to generate sequence reads;
(d) selecting a set of sequence reads in which the microsatellite exceeds a disruption index so that the error rate between matched pairs is less than 2%, less than 1%, or preferably less than 0.5%, wherein matched pairs are independent reads that share a template, first copy, or locus; and
(e) measuring microsatellite lengths from the set of sequence reads selected in step (d), wherein the measurement is the distance between the flanking portions that delineate the microsatellite of the initial templates.

In embodiments, as an alternative to the step of “selecting a set of sequence reads in which the microsatellite has been disrupted by mutagenesis so that (i) at least two, preferably more of the tandem repeat units in the microsatellite have been mutagenized, and (ii) the microsatellite in the sequence read has no more than 7, preferably 5 or fewer, tandem repeats in a row” in the methods described herein, the step instead comprises selecting a set of sequence reads selects reads in which the microsatellite has been disrupted by mutagenesis sufficiently to reduce the replication error rate of the disrupted microsatellite to 10⁻¹ or less, preferably 10⁻² or less, or more preferably 10⁻³ or less. The error rate in this context is the proportion of duplicates that deviate from the actual microsatellite length. Error rates for a given degree of disruption can be estimated, for example, using a look-up table of disruption and error rates that was generated using synthetic templates of known composition and length.

Accordingly, this invention provides a method for measuring microsatellite lengths of initial nucleic acid templates each of which comprise a microsatellite and two flanking portions, which flanking portions delineate the microsatellite, the method comprising:
(a) generating partially mutagenized templates by:
   (i) partial mutagenesis of the initial nucleic acid templates;
   (ii) partial mutagenesis during production of first copies of the initial nucleic acid templates; and/or
   (iii) generating first copies of the initial nucleic acid templates followed by partial mutagenesis of the first copies of the initial nucleic acid templates;
(b) making a sequencing library from the partially mutagenized templates;
(c) sequencing the library from step (b) to generate sequence reads;
(d) selecting a set of sequence reads in which the microsatellite has been disrupted by mutagenesis sufficiently to reduce the replication error rate of the disrupted microsatellite to 10⁻² or less; and
(e) measuring microsatellite lengths from the set of sequence reads selected in step (d), wherein the measurement is the distance between the flanking portions that delineate the microsatellite of the initial templates.

### Partial Mutagenesis

In embodiments, the partial mutagenesis comprises:
(a) chemical mutagenesis;
(b) enzymatic mutagenesis;
(c) incorporating nonstandard nucleotides during a step of replication; or
(d) combinations thereof.

In embodiments, the partial mutagenesis comprises treating the initial nucleic acid template or a first copy of the initial nucleic acid template with an enzyme that deaminates nucleotides.

In embodiments, the enzyme that deaminates nucleotides is adenine deaminase.

In embodiments, the partial mutagenesis comprises deamination of cytosines, preferably deamination of cytosines by bisulfite mutagenesis.

In embodiments, the partial mutagenesis comprises nick translation of the initial nucleic acid template or a first copy of the initial nucleic acid template to replace nucleotides of the template with nonstandard nucleotides having altered base-pairing activity.

In embodiments, the partial mutagenesis comprises copying the initial nucleic acid templates or first copies of the initial nucleic acid templates in the presence of a mixture of standard nucleotides and nonstandard nucleotides to generate copies comprising standard and nonstandard nucleotides, wherein the nonstandard nucleotides have altered base-pairing activity.

In embodiments, the nonstandard nucleotides are deoxyinosine triphosphate.

In embodiments, the partial mutagenesis comprises:
(a) copying the initial nucleic acid templates or first copies of the initial nucleic acid templates in the presence of a mixture of standard nucleotides and nonstandard nucleotides to generate copies comprising standard and nonstandard nucleotides;
(b) subjecting the copies comprising standard and nonstandard nucleotides to a chemical or enzymatic treatment that alters the base-pairing activity of a standard nucleotide without altering the base-pairing activity of its corresponding nonstandard nucleotide.

In embodiments, the nonstandard nucleotides are 5-methylcytosine

In embodiments, the chemical or enzymatic treatment comprises:
(a) using a TET2 enzyme to oxidize 5-methylcytosine into 5-carboxycytosine; and
(b) using an APOBEC enzyme to convert cytosines to uracils.

In embodiments, the partial mutagenesis is followed by production of first copies in the presence of a mixture of 5-methyl-dCTP and standard nucleotides, preferably wherein 5-methyl-dCTP is present in the mixture at about the same concentration as dCTP.

In embodiments, the partial mutagenesis comprises:
(a) copying the initial nucleic acid templates or first copies of the initial nucleic acid templates in the presence of a mixture of standard nucleotides and nonstandard nucleotides to generate copies comprising standard and nonstandard nucleotides;
(b) subjecting the copies comprising standard and nonstandard nucleotides to a chemical or enzymatic treatment that alters the base-pairing activity of the nonstandard nucleotide without altering the base-pairing activity of its corresponding standard nucleotide.

In embodiments, the method comprises a step of protecting the flanking portions of the initial nucleic acid templates or first copies of the initial nucleic acid templates from partial mutagenesis.

In embodiments, protecting the flanking portions of the initial nucleic acid templates or first copies of the initial nucleic acid templates comprises using an excess of oligonucleotides complementary to the flanking portions to protect the flanking portions from partial mutagenesis, preferably wherein the partial mutagenesis comprises deamination of cytosines, preferably deamination of cytosines by bisulfite mutagenesis.

### Initial Templates

### Sources

In embodiments, the initial nucleic acid templates:
(a) are from a biological sample;
(b) are copies of nucleic acids from a biological sample; or
(c) are synthetic templates.

In embodiments, the initial nucleic acid templates are from a biological sample or are copies of nucleic acids from a biological sample, and the biological sample is:
(a) from a tissue biopsy;
(b) from blood or a blood product;
(c) from excreta, preferably urine or fecal matter; or
(d) sputum.

In embodiments, the initial nucleic acid templates are synthetic templates, wherein the synthetic templates each comprise two flanking portions and a microsatellite of known composition and length, each optionally comprising a varietal tag, a sample barcode, and/or a universal primer binding site, wherein the microsatellite:
(a) comprises nucleotides susceptible to being altered by the step of partial mutagenesis; or
(b) comprises a known pattern of mutation.

### Fragmentation

In embodiments, the initial nucleic acid templates were prepared by random fragmentation of nucleic acids.

In embodiments, the random fragmentation was by:
(a) a natural process, preferably degradation of nucleic acids;
(b) shearing; and/or
(c) endonucleases.

In embodiments, the initial nucleic acid templates were prepared by restriction endonuclease cleavage of nucleic acids

### Enrichment

In embodiments, the initial nucleic acid templates are in a sample that has been enriched for microsatellites by a panel comprising oligonucleotides with sequence complementarity to:
(a) one or more microsatellite flanking portions; or
(b) a microsatellite repeat motif.

In embodiments, the initial nucleic acid templates are in a sample enriched for microsatellites and the method comprises a step of enriching a sample comprising a population of nucleic acid templates for microsatellites using a panel comprising oligonucleotides with sequence complementarity to:
(a) one or more microsatellite flanking portions;
(b) a microsatellite repeat motif.

In embodiments, step (a) comprises a step of enriching partially mutagenized templates for microsatellites using a panel comprising oligonucleotides with sequence complementarity to:
(a) one or more microsatellite flanking portions; or
(b) a microsatellite repeat motif.

In embodiments, the panel is:
(a) a panel of hybridization capture probes; or
(b) a panel of primers to initiate replication.

### Individual Identifiers

In embodiments, the initial nucleic acid templates and/or first copies thereof comprise one or more individual identifiers.

In embodiments, the one or more individual identifiers comprise:
(a) fragment end sequences if the initial nucleic acid template is from a biological sample and is randomly fragmented;
(b) fragment end sequences if the initial nucleic acid template was prepared using random fragmentation;
(c) a mutational pattern caused by the step of partial mutagenesis, wherein the step of partial mutagenesis is partial random mutagenesis;
(d) a varietal tag attached to the initial nucleic acid templates or first copies of the initial nucleic acid templates;
(e) a sequence of the flanking portions of the microsatellite, which:
   (i) specify the locus of the microsatellite in a reference genome if the initial nucleic acid template is from a biological sample; or
   (ii) specify a synthetic nucleic acid molecule if the initial nucleic acid template is a synthetic template; or
(f) any combination of the above.

### Adaptors

In embodiments, the initial nucleic acid templates comprise one or more adaptors, preferably wherein the one or more adaptors convey template identity and/or sample identity.

In embodiments, the one or more adaptors comprise one or more or all of the following:
(a) a varietal tag;
(b) a sample barcode;
(c) a universal primer binding site;
(d) a purification moiety, preferably biotin; and
(e) a sequencing primer binding site.

In embodiments, the one or more adaptors consist of nucleotides that are:
(a) not susceptible to being altered in the step of partial mutagenesis; or
(b) complementary to nucleotide that are not susceptible to being altered in the step of partial mutagenesis.

In embodiments, the one or more adaptors are added to the initial nucleic templates, first copies of the initial nucleic acid templates, and/or partially mutagenized copies of the initial nucleic acid templates by:
(a) ligation; or
(b) primer extension.

In embodiments, the one or more adaptors have a fish-tail structure.

### First Copies

In embodiments, step (a), part (i) comprises generating first copies of the partially mutagenized templates.

In embodiments, one or more adaptors are added to the first copies.

In embodiments, the one or more adaptors comprise one or more or all of the following:
(a) a varietal tag;
(b) a sample barcode;
(c) a universal primer binding site; and
(d) a purification moiety, preferably biotin.

### Preparing Sequencing Libraries

In embodiments, step (b) comprises amplification of the partially mutagenized templates.

In embodiments, amplification comprises linear amplification, exponential amplification, or both.

In embodiments, amplification is with:
(a) a DNA polymerase;
(b) a RNA polymerase; or
(c) a reverse transcriptase.

In embodiments, amplification is with primers consisting of nucleotides that are not susceptible to being altered in the step of partial mutagenesis.

In embodiments, the partially mutagenized templates comprise a purification moiety and step (b) comprises purifying the partially mutagenized templates using the purification moiety, preferably prior to a step of exponential amplification, preferably wherein the purification moiety is biotin and the partially mutagenized templates are purified by binding of the purification moiety to streptavidin.

In embodiments, step (b) comprises enriching the partially mutagenized templates for microsatellites, before or after amplification.

In embodiments, enriching is with a panel comprising oligonucleotides with sequence complementarity to:
(a) one or more microsatellite flanking portions; or
(b) a microsatellite repeat motif.

In embodiments, the panel is:
(a) a panel of hybridization capture probes; or
(b) a panel of primers to initiate replication.

In embodiments, amplification is by polymerase chain reaction (PCR).

In embodiments, step (b) comprises end-polishing, A-tailing, and sequencing adaptor ligation.

### Sequence Reads

In embodiments, the sequence reads:
(a) are single reads or paired end reads;
(b) have sample barcode sequences; and/or
(c) have varietal tag sequences.

### Microsatellites

In embodiments, the microsatellites:
(a) are at least four repeat units in length;
(b) comprise repeat units, each of which is no more than 10 nucleotides;
(c) are at least 12 nucleotides in length;
(d) are mononucleotide tracts, preferably mono-C tracts;
(e) are dinucleotide tracts, preferably C/G tracts or C/A tracts;
(f) comprise cytosines;
(g) comprise adenines;
(h) are susceptible to a method of partial mutagenesis;
(i) are known to have unstable replication;
(j) are more than 5 repeat units in length, more than 7 repeat units in length, more than 10 repeat units in length, more than 15 repeat units in length, more than 20 repeat units in length, more than 30 repeat units in length, between 6 and 70 repeat units in length, between 6 and 32 repeat units in length or between 12 and 64 repeat units in length; and/or
(k) are from a genome of an organism and adjoin flanking portions in the genome of the organism, wherein a flanking portion together with the microsatellite map uniquely to the genome to define the locus of the microsatellite, and wherein the flanking portions delineate the length of the microsatellite.

### Provenance

In embodiments, the method comprises establishing one or more provenances of the sequence reads.

In embodiments, the one or more provenances are:
(a) a locus in a reference genome and the provenance is established using a sequence in one or both of the flanking portions;
(b) a synthetic nucleic acid template and the provenance is established using a sequence in one or both of the flanking portions;
(c) an initial nucleic acid template with a specific individual identifier and the provenance is established using said individual identifier;
(d) a first copy of an initial nucleic acid template with a specific individual identifier and the provenance is established using said individual identifier;
(e) a partially mutagenized template with a specific individual identifier and the provenance is established using said individual identifier;
(f) a partially mutagenized first copy of an initial nucleic acid template with a specific individual identifier and the provenance is established using said individual identifier;
(g) a sample with a specific sample barcode and the provenance is established using said sample barcode; and/or
(h) a partially mutagenized template with a specific degree of microsatellite disruption preferably wherein the provenance is established based a common maximum repeat length and/or a common proportion of mutagenized bases.

In embodiments, the individual identifier comprises:
(a) fragment end sequences if the initial nucleic acid template is from a biological sample and is randomly fragmented;
(b) fragment end sequences if the initial nucleic acid template was prepared using random fragmentation;
(c) a mutational pattern caused by the step of partial mutagenesis, wherein the step of partial mutagenesis is partial random mutagenesis;
(d) a varietal tag attached to the initial nucleic acid templates or first copies of the initial nucleic acid templates;
(e) a sequence of the flanking portions of the microsatellite, which
   (i) specify the locus of the microsatellite in a reference genome if the initial nucleic acid template is from a biological sample; or
   (ii) specify a synthetic nucleic acid molecule if the initial nucleic acid template is a synthetic template; or
(f) any combination of the above.

### Distributions

In embodiments of the method for obtaining a distribution of microsatellite lengths from initial nucleic acid templates, step (c) or (d) comprises applying a consensus rule to the sequence reads or set of selected sequence reads, so that the distribution of measured microsatellite lengths is from:
(a) a set of consensus lengths identified as originating from the same first copies;
(b) a set of consensus lengths identified as originating from the same initial nucleic acid templates;
(c) a set of consensus lengths identified as originating from the same first copies and initial nucleic acid templates.

In embodiments, the consensus rule assigns a microsatellite length to sequence reads originating from the same first copy or initial nucleic acid template if:
(a) a microsatellite length is agreed upon by at least P % of sequence reads originating from the same first copy or initial nucleic acid template, wherein there is no microsatellite length in said sequence reads originating from the same first copy or initial nucleic acid template found more frequently that P %, preferably wherein P is a value from 30 to 100;
(b) a microsatellite length is agreed upon by a plurality of sequence reads originating from the same first copy or initial nucleic acid template; or
(c) a microsatellite length is agreed upon by all sequence reads originating from the same first copy or initial nucleic acid template.

In embodiments, sequence reads are identified as originating from the same initial nucleic acid template or first copy of initial template nucleic acid template based on:
(a) fragment end sequences;
(b) a mutational pattern caused by the step of partial mutagenesis, if the partial mutagenesis is partial random mutagenesis; or
(c) one or more varietal tags attached to the initial nucleic acid templates or first copies of the initial nucleic acid templates.

In embodiments, the distribution of microsatellite lengths in the initial nucleic acid templates consists of:
(a) a distribution of microsatellite lengths at an individual locus;
(b) a set of distributions of microsatellite lengths at two or more individual loci.

In embodiments of the methods of measuring microsatellite lengths, the method further comprises generating a distribution of microsatellite read lengths by counting the number of microsatellites of a given length across all measured microsatellite lengths in a set of sequence reads having a shared provenance, wherein the shared provenance is selected from the group consisting of:
(a) sample;
(b) locus;
(c) synthetic template;
(d) initial template identity;
(e) first copy identity; or
(f) degree of disruption.

In embodiments, the method comprises generating a distribution of consensus microsatellite lengths, wherein:
(a) the consensus microsatellite lengths derive from the distribution of microsatellite read lengths over a set of identified templates by applying a consensus rule;
(b) the consensus microsatellite lengths derive from the distribution of microsatellite read lengths over a set of identified first copies by applying a consensus rule; or
(c) the consensus microsatellite lengths derive from a distribution of consensus microsatellite lengths over a set of identified first copies sharing a set of identified initial nucleic acid templates by applying a consensus rule.

In embodiments, the consensus rule is chosen from the group consisting of:
(a) a unanimity rule, in which the consensus microsatellite length is the only microsatellite length in the distribution and all other microsatellite lengths have a count of zero;
(b) a plurality rule, in which the consensus microsatellite length is the most common microsatellite length in the distribution;
(c) the majority P-rule, in which the consensus microsatellite length is the microsatellite length with a count that is greater than or equal to N×P where N is the total number of microsatellite lengths and P is greater than or equal to 0.5.

In embodiments:
(a) the sequencing library is enriched for microsatellites by a panel comprising oligonucleotides with sequence complementarity to one or more microsatellite flanking portions at one or more loci and optionally comprises synthetic nucleic acid templates of known microsatellite composition, length, and degree of disruption; and
(b) distributions of microsatellite lengths at one or more loci are generated by:
   (i) using an exact matching algorithm to identify matches between the sequence reads and an alignment index database, wherein the alignment index database comprises, for each locus corresponding to a microsatellite of the panel and for each synthetic nucleic acid template, if present:
      (1) a subsequence for each flanking portion and all possible variations that can arise from partial mutagenesis; and
      (2) the distance of each said subsequence to the microsatellite;
   (ii) for each sequence read that matches a subsequence of the alignment index database measuring the microsatellite length using the distance in the alignment index database; and
   (iii) counting the number of microsatellites of a given length across all measured microsatellite lengths in a set of sequence reads having a shared provenance, wherein the shared provenance is selected from the group consisting of:
      (1) sample;
      (2) locus;
      (3) synthetic template;
      (4) initial template identity;
      (5) first copy identity; or
      (6) degree of disruption.

### Look-Up Tables

In embodiments, the step of selecting a set of sequence reads comprises:
(a) using a look-up table of disruption and error rates to estimate the replication error associated with a given pattern of disruption in a sequence read;
(b) selecting sequence reads based on the estimated replication error.

In embodiments, the look-up table of disruption and error rates was generated using synthetic templates that match the microsatellite tracts of the initial templates in composition and length.

In embodiments, the look-up table of disruption and error rates was generated by:
(a) preparing distributions of read lengths and consensus read lengths of synthetic nucleic acid templates of known microsatellite composition, length, and degree of disruption;
(b) from the distributions of read lengths and consensus read lengths, estimating an error rate per round of replications as a function of the degree of disruption;
(c) from the estimated error rate per round of replication, generating a look-up table of the moments of error as a function of the degree of disruption and number of rounds of replication.

Accordingly, this invention provides a method for generating a look-up table to estimate the replication error associated with a given pattern of disruption in a sequence read.

### Reports

In embodiments, the methods described herein further comprise a step of generating a report based on the measured microsatellite lengths. A person skilled in the art will appreciate that such a report may include any information that is produced by the methods described herein. For example, where applicable and without limitation, the report may include the measured microsatellite lengths at one or more loci and the confidence interval of these measurements.

Accordingly, this invention also provides reports produced according the methods described herein.

### Applications

This invention also provides a method for obtaining a distribution of microsatellite lengths of nucleic acid templates from two or more samples derived from the same organism, the method comprising:
(a) performing any of the methods for obtaining a distribution of microsatellite lengths described herein on the two or more samples derived from the same organism to thereby determine the distribution of microsatellite lengths in initial nucleic acid templates in each of the two or more samples; and
(b) combining the distribution of microsatellite lengths in initial nucleic acid templates in each of the two or more samples.

In embodiments, the two or more samples are derived from:
(a) different tissues of the organism; and/or
(b) the same tissue of the organism sampled at different times.

This invention also provides a method for detecting microsatellite length variation (MSLV) in initial nucleic acid templates of a first sample derived from an organism:
(a) performing any of the methods for obtaining a distribution of microsatellite lengths described herein on the sample to thereby determine the distribution of microsatellite lengths in initial nucleic acid templates in the first sample; and
(b) comparing the distribution of microsatellite lengths in initial nucleic acid templates in the first sample to a reference distribution, wherein the reference distribution was produced from a sample derived from the same organism according to the same method.

In embodiments, the reference distribution was produced from a sample derived from a different tissue of the organism than the first sample.

In embodiments, the comparison of distributions is used to infer the somatic MSLV of the first sample.

In embodiments, the first sample, or the sample from which the reference distribution was produced, is derived from a neoplasm.

In embodiments, the reference distribution was produced from a sample derived from the same tissue of the organism as the first sample, sampled at a different time.

In embodiments, the comparison of distributions is used to determine residual neoplasm following therapy.

In embodiments, the comparison of distributions is used to detect the presence or absence of clonal growth in the tissue.

In embodiments, the clonal growth is a previously undetected neoplasm.

A method for detecting microsatellite length variation (MSLV) in initial nucleic acid templates of two or more samples derived from the same organism, the method comprising:
(a) performing any of the methods for obtaining a distribution of microsatellite lengths described herein on the two or more samples derived from the same organism to thereby determine the distribution of microsatellite lengths in initial nucleic acid templates in each of the two or more samples; and
(b) comparing the distribution of microsatellite lengths in initial nucleic acid templates in each of the two or more samples.

In embodiments, the two or more samples:
(a) are derived from different tissues of the organism; and/or
(b) are derived from the same tissue of the organism sampled at different times.

In embodiments, the two or more samples are derived from different tissues of the organism and the comparison of distributions is used to infer the somatic MSLV of each sample.

In embodiments, one of the two or more samples is derived from a neoplasm.

In embodiments, the two or more samples are derived from the same tissue of the organism sampled at different times and the comparison of distributions is used to determine residual neoplasm following therapy.

In embodiments, the two or more samples are derived from the same tissue of the organism sampled at different times and the comparison of distributions is used to detect the presence or absence of clonal growth in the tissue.

In embodiments, the clonal growth is a previously undetected neoplasm.

A method for detecting the presence or absence of clonal populations of cells in a tissue of an organism, the method comprising performing any of the methods for obtaining a distribution of microsatellite lengths described herein on a sample derived from a single tissue of the organism to detect the presence or absence of minor variant microsatellite lengths, wherein the presence or absence of minor variant microsatellite lengths indicate the presence or absence of a clonal population of cells.

In embodiments, the clonal population of cells is a previously undetected neoplasm.

In embodiments, the comparison is used to detect the presence or absence of a disease in the organism.

This invention also provides a method for detecting the presence or absence of a rare microsatellite length (MSL) in a population, the method comprising:
(a) performing any of the methods for obtaining a distribution of microsatellite lengths described herein on a sample derived from an individual in the population to thereby determine the distribution of microsatellite lengths in initial nucleic acid templates in the sample;
(b) comparing the distribution of microsatellite lengths in initial nucleic acid templates in the sample to a distribution of microsatellite lengths in the population that was prepared by the same method to thereby detect the presence or absence of a rare MSL.

In embodiments, the method is used to detect a rare MSL in:
(a) sperm cells;
(b) eggs;
(c) embryos;
(d) a fetus;
(e) a biological sample of unknown origin;
(f) a population of biological samples, preferably wherein the population of biological samples is mass-produced;
(g) animals;
(h) gametes of animals
(i) plants;
(j) gametes of plants;
(k) livestock;
(l) gametes of livestock;
(m) crops; or
(n) gametes of crops.

This invention also provides a method of selective breeding comprising:
(a) using the method of detecting a rare MSL on one or more individuals in a population to detect the presence or absence of a rare MSL in the one or more individuals of the population;
(b) selecting an individual in the population based on the presence or absence of a rare MSL detected in step (a); and producing one or more individuals or one or more generations of individuals from reproductive material of the individual selected in step (b).

In embodiments, the method comprises estimating a confidence interval for the proportion of initial templates with a microsatellite length L at a single locus in a single sample, wherein the confidence interval is estimated using the distribution of measured microsatellite lengths in reads with a given degree of disruption and a look-up table of disruption and error rates.

In embodiments, the look-up table of disruption and error rates was generated using synthetic templates that match the microsatellite tracts of the initial templates in composition and length.

In embodiments, the look-up table of disruption and error rates was generated by:
(a) preparing distributions of read lengths and consensus read lengths of synthetic nucleic acid templates of known microsatellite composition, length, and degree of disruption;
(b) from the distributions of read lengths and consensus read lengths, estimating an error rate per round of replications as a function of the degree of disruption;
(c) from the estimated error rate per round of replication, generating a look-up table of the moments of error as a function of the degree of disruption and number of rounds of replication.

In embodiments, the confidence interval is used:
(a) to determine the profile of microsatellite length variation in a tumor;
(b) to genotype an individual;
(c) to detect disease loci;
(d) for early detection of cancer; or
(e) to determine the health of a sampled tissue.

This invention also provides a method of comparing two or more samples over one or more microsatellite loci for microsatellites of length L comprising:
(a) estimating a confidence interval for each sample, each locus, and each microsatellite length L according to any of the above methods; and
(b) comparing the estimated confidence intervals.

In embodiments, the two or more samples are from:
(a) different tissues of the same person, preferably a tumor biopsy and a blood sample;
(b) same tissues sampled at different times, preferably blood, before and after a treatment;
(c) same tissue, fractionated into components, preferably blood cells and cell-free components of blood; or
(d) different persons, preferably for forensics, parentage determination, and population studies.

This invention also provides a method of measuring deviation of microsatellite lengths at one or more loci in a sample relative to microsatellite lengths (A, B) at the one or more loci of a mono-allelic or bi-allelic baseline sample, the method comprising:
(a) determining a distribution of microsatellite lengths P(l) in the sample at each of the one or more loci according to any of the methods described herein for determining a distribution of microsatellite lengths,
(b) calculating a K-eccentricity E_{K}(A,B,P) of the sample to the baseline sample at each of the one or more loci, wherein for a given locus with baseline microsatellite lengths (A, B)

$${E}_{K} ( A , B , P ) = {\sum }_{l} P ( l ) min {( \vert A - l \vert , \vert B - l \vert )}^{K}$$

wherein K is a positive integer and the K-eccentricity E_{K}(A,B,P) is a measure of the deviation of microsatellite lengths at each locus in the sample relative to the microsatellite lengths (A, B) at the locus of a mono-allelic or bi-allelic baseline sample.

In embodiments of the invention, the mono-allelic or bi-allelic baseline sample is a germline sample. In embodiments of the invention, the sample is a blood sample, preferably blood cells or cell-free component of the blood sample. In embodiments of the invention, the sample is a sample from a tumor.

In embodiments of the invention, the method further comprises:
(a) calculating the mean K-eccentricity of all mono-C and all d-AC loci of the sample for which a distribution of microsatellite lengths P(l) was determined; and/or
(b) calculating the mean K-eccentricity of all mono-C and all d-AC loci that exhibit low eccentricity in a normal sample.

### Sequencing Libraries, Composition of Matter

This invention also provides a sequencing library comprising nucleic acid templates, wherein at least 5%, preferably at least 10%, more preferably at least 20%, and more preferably at least 30% of the nucleic acid templates comprise:
(a) a mutagenized microsatellite in which:
   (i) at least two, preferably more of the tandem repeat units in the microsatellite have been mutagenized; and
   (ii) the mutagenized microsatellite has no more than 7, preferably 5 or fewer, tandem repeats in a row; and
(b) two flanking portions.

In embodiments, the sequencing library comprises at least 100, at least 1,000, at least 1×10⁴, at least 3×10⁴, or at least 6×10⁴ nucleic acid templates.

In embodiments, the sequencing library was enriched from a whole genome.

This invention also provides a sequencing library comprising nucleic acid templates that have been partially mutagenized according to any of the methods of directed partial mutagenesis described herein, i.e. the methods of partial mutagenesis in which the flanking portions are protected from partial mutagenesis.

This invention also provides a sequencing library comprising nucleic acid templates which comprise:
(a) a mutagenized microsatellite in which:
   (i) at least two, preferably more of the tandem repeat units in the microsatellite have been mutagenized; and
   (ii) the mutagenized microsatellite has no more than 7, preferably 5 or fewer, tandem repeats in a row; and
(b) flanking portions which are not mutagenized.

This invention also provides a whole genome sequencing library comprising nucleic acid templates according to the above two embodiments, i.e. where the nucleic acid templates of the whole genome sequencing library have been subjected to directed partial mutagenesis.

### Panels, Compositions of Matter

This invention also provides a panel comprising a set of 6 or more oligonucleotides with sequences complementary to the flanking portions of microsatellites that are susceptible to the partial mutagenesis described in any of the above embodiments.

In embodiments:
(a) the microsatellites are at least four repeat units in length;
(b) the microsatellites comprise repeat units, each of which is no more than 10 nucleotides;
(c) the microsatellites are at least 12 nucleotides in length;
(d) the microsatellites are mononucleotide tracts, preferably mono-C tracts;
(e) the microsatellites are dinucleotide tracts, preferably C/G tracts or C/A tracts;
(f) the microsatellites comprise cytosines;
(g) the microsatellites comprise adenines;
(h) the microsatellites are known to have repeat length variability;
(i) the microsatellites are more than 5 repeat units in length, more than 7 repeat units in length, more than 10 repeat units in length, more than 15 repeat units in length, more than 20 repeat units in length, more than 30 repeat units in length, between 6 and 70 repeat units in length, between 6 and 32 repeat units in length or between 12 and 64 repeat units in length;
(j) the microsatellite comprises a flanking portion which, together with the microsatellite, maps uniquely to the genome; and/or
(k) the oligonucleotides are complementary to flanking portions that do not hybridize to other flanking portions.

In embodiments, the panel is:
(a) a panel of hybridization capture probes; or
(b) a panel of primers to initiate replication.

In embodiments of the panel, the sequences of the oligonucleotides are complementary to one or more of the sequences set forth in SEQ ID NOs: 1-1260 and 1891-3150.

### Kits

This invention also provides a kit for performing the methods described herein.

In embodiments, the kit comprises the panels described herein.

In embodiments, the kit further comprises one or more or all of the following:
(a) synthetic nucleic acid templates, each comprising two flanking portions and a microsatellite of known composition and length, each optionally comprising a varietal tag, a sample barcode, and/or a universal primer binding site, wherein the microsatellite:
   (i) comprises nucleotides susceptible to being altered by a step of partial mutagenesis; or
   (ii) comprises a known pattern of mutation;
(b) oligonucleotide adaptors, optionally fish-tail adaptors, each of which comprise one or more of the following:
   (i) a varietal tag;
   (ii) a sample barcode;
   (iii) a universal primer binding site; and
   (iv) a purification moiety, preferably biotin;
(c) primers to initiate replication, wherein the primers are complementary to:
   (i) a universal primer binding site;
   (ii) a flanking portion of a synthetic nucleic acid template;
   (iii) a flanking portion of an initial nucleic acid template, which initial nucleic acid templates comprise a microsatellite and two flanking portions, optionally wherein the sequences of the primers are complementary to one or more of the sequences set forth in SEQ ID NOs: 1-1260 and 1891-3150; and/or
   (iv) a flanking portion of a first copy of an initial nucleic acid template, which initial nucleic acid templates comprise a microsatellite and two flanking portions; and
(d) a set of oligonucleotide blockers complementary to flanking portions of the microsatellites of the panel, optionally wherein the sequences of the oligonucleotide blockers are complementary to one or more of the sequences set forth in SEQ ID NOs: 1-1260 and 1891-3150;
wherein the oligonucleotide adaptors and primers to initiate replication preferably consist of nucleotides which (1) are not susceptible to being altered by a step of partial mutagenesis, and/or (2) are complementary to nucleotides which are not susceptible to being altered by a step of partial mutagenesis.

In embodiments, the kit further comprises:
(a) enzymes or chemicals for partial mutagenesis of nucleic acid templates; and/or
(b) computer-readable media comprising:
   (i) an alignment index database comprising, for each locus corresponding to a microsatellite of the panel of the kit, and for each synthetic nucleic acid template of the kit, if present:
      (1) a subsequence for each flanking portion and all possible variations that can arise from partial mutagenesis; and
      (2) the distance of each said subsequence to the microsatellite; and
   (ii) software for matching sequence reads to subsequences of the alignment index database.

In embodiments, the kit comprises:
(a) double-stranded synthetic nucleic acid templates, comprising a flanking portion complementary to a sequence of the panel;
(b) single-stranded synthetic nucleic acid templates, each comprising a microsatellite comprising nucleotides susceptible to being altered by a step of partial mutagenesis; and
(c) single-stranded synthetic nucleic acid templates, each comprising a microsatellite with a known pattern of mutation.

### Alternatives

A person skilled in the art will readily appreciate that an equivalent alternative to generating partially mutagenized templates for the purposes of the methods described herein is to obtain partially mutagenized templates that have been prepared by partial mutagenesis as described herein. Accordingly, for each embodiment of the invention which includes a step of generating partially mutagenized templates, there is an alternative embodiment wherein the step of generating partially mutagenized templates is replaced by a step of obtaining (for example from a third party) partially mutagenized templates that were produced by partial mutagenesis.

Similarly, a person skilled in the art will readily appreciate that an equivalent alternative to making a sequencing library for the purposes of the methods described herein is to obtain a sequencing library comprising mutagenized templates wherein said mutagenized templates are ready for sequencing (i.e. the sequencing library comprises mutagenized templates that have been replicated sufficiently to be sequenced and may comprise sequencing-platform specific adaptors). Accordingly, for each embodiment of the invention which includes a step of making a sequencing library, there is an alternative embodiment wherein the step of making a sequencing library is replaced by a step of obtaining a sequencing library comprising mutagenized templates (for example from a third party), followed by sequencing of the sequencing library.

Further, a person skilled in the art will readily appreciate that an equivalent alternative to sequencing the sequencing library for the purposes of the methods described herein is to obtain sequence reads of the sequencing library (for example from a third party). Accordingly, for each embodiment of the invention which includes a step of sequencing a sequencing library, there is an alternative embodiment wherein the step of sequencing the sequencing library is replaced by a step of obtaining sequence reads from a sequencing library.

In an embodiment, the method comprises a step of generating a distribution of measured microsatellite lengths from the selected set of sequence reads at an individual locus. As discussed herein, this distribution is representative of the distribution of microsatellite lengths in the initial nucleic acid templates at that individual locus. In an embodiment, the method comprises a step of generating a set of distributions of measured microsatellite lengths at two or more individual loci from the selected set of sequence reads. As discussed herein, each distribution in the set of distributions is representative of the distribution of microsatellite lengths in the initial nucleic acid templates at each given individual locus.

In an embodiment, the method comprises determining the degree of disruption of the tandem repeat structure in the sequence reads prior to the step of selecting a set of sequence reads.

### Terms and Concepts

Unless otherwise defined, all technical and scientific terms used herein have the same meaning as commonly understood by a person of ordinary skill in the art to which this invention belongs. As used herein, and unless stated otherwise, each of the following terms shall have the definition set forth below.

About: In the context of a numerical value or range means±10% of the numerical value or range recited or claimed, unless the context requires a more limited range.

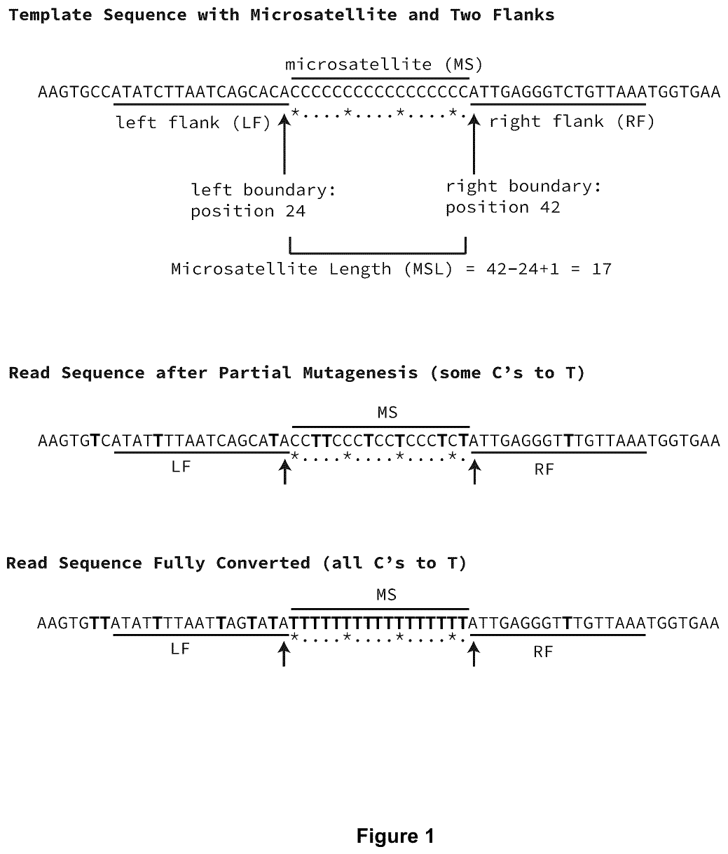

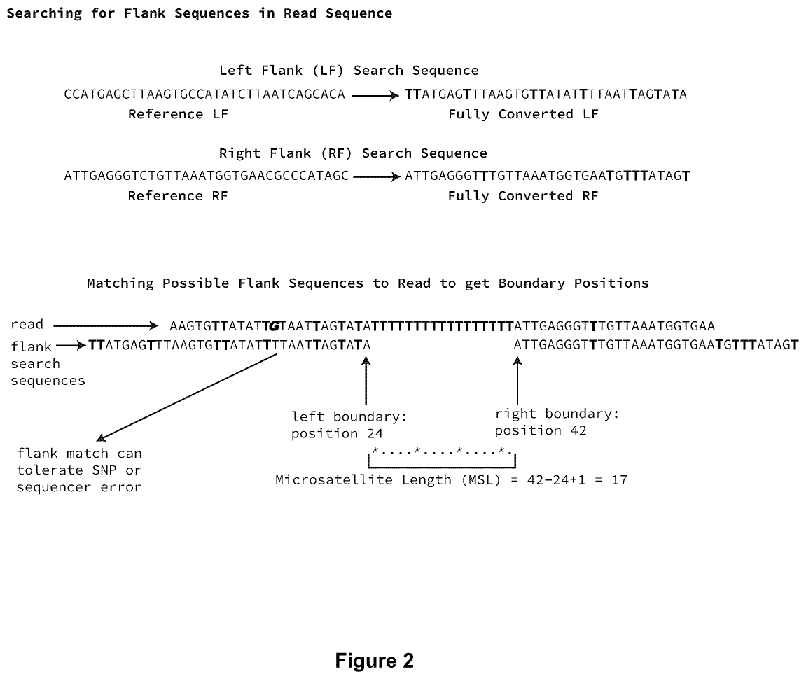

Microsatellite: A microsatellite (MS) is a portion of the genome comprised of tandem repeats of a simple sequence. An MS is characterized by that repeat unit, the number of repeat units, and the total length which equals the number of repeats times the length of the repeat. For example in the FIG. 1, the microsatellite is a mononucleotide repeat, of 17 units, and a total length of 17. Each microsatellite has flanking sequences that place a given MS at a specific locus in the genome. The distance between the flanking sequences determines the microsatellite length (see FIG. 2). There are millions of microsatellites in the genome of complex organisms such as humans. Avvaru, A. K., et al., 2020 provides a database of microsatellites in the human genome, and the genomes of other organisms, which can be searched by repeat unit, location and flanking sequence. The database may be found at the following top-level domain: data.ccmb.res.in.

Biological variability of microsatellites: The tandem repeat structure of a microsatellite gives rise to high variability during replication, most frequently losses or gains of some number of the repeat unit. Microsatellites replicate during new cell growth, and as such, in a population of organisms or cells, the microsatellite lengths over any given locus form a distribution reflecting the history of that population. Variations in microsatellite length can be used to distinguish individual organisms, useful in forensics, genetic testing or breeding. Due to their instability, variations in microsatellite lengths can also cause genetic disorders (i.e. Huntington's Disease (Snell, R. G., et al. 1993), fragile X syndrome (Verkerk, A. J. et al. 1991), myotonic dystrophy (Ranum, L. P. & Day, J. W., 2002), and other neurological disorders (Brouwer, J. R., et al., 2009)), and are associated with proliferative genetic disorders, such as neoplasms (Fujimoto, A., et al., 2020; Bonneville, R. et al., 2017; Hause, R. J. et al., 2016; Kim, T. M., et al., 2013). Monitoring rare microsatellite variation could facilitate de novo detection of any new clonal expansion, from early detection of cancer to clonal hematopoiesis (Jaiswal, S. & Ebert, B. L, 2019), or clonal expansions in the germline (Goriely, A. & Wilkie, 2012).

Biological sample: Any material, including without limitation, blood, serum, fluid biopsy samples, tissue biopsy samples, and forensic samples, that originates from a biological source.

Disrupting the microsatellite repeat structure by partial mutagenesis: The same property that creates variable microsatellite length during replication makes it difficult to assay these markers following exponential amplification. The approach described herein is to disrupt the microsatellite repeat structure sufficiently during the processing of templates in a biological sample prior to their exponential amplification, to stabilize their length, and allow detection of length variation in the biological sample with accuracy and sensitivity. The method uses partial mutagenesis of templates or their first copies that can alter the repeat unit, thereby reducing the number of tandem repeats while not changing the original distance between the microsatellite flanks. But if mutagenesis is not partial, but complete or nearly complete, then a new tandem repeat structure as unstable as the original structure can be created. Therefore, disruption of a tandem repeat structure is the criterion for creating a stable replicating molecule. Typically, the degree of disruption is determined for each sequence by the presence of sequence alterations within the tandem repeats that would be expected from the mutagenesis.

Replicative error during the first round and during exponential amplification: Microsatellite length is not stable under most conditions of replication. It is hypothesized that is due to polymerase slippage, because the changes in length typically come in units of the length of the tandem repeat that make up the microsatellite (Clarke, L. A., et al., 2001, Kunkel, T. A., 1986). Because the error in a read is dependent on the number of rounds of replication, it is useful to refer to the replication error rate as error per round of replication. As gain in length is less common than loss of length, it is also useful to specify the nature of the length error, for example, as a loss of one unit per round. There are two ways to estimate the replication error: assess the number of first round copies of a template of known length that are in error; or model the number of reads that are in error after N rounds of replication. If the latter is after N rounds of exponential amplification, then the proper model is that derived from Luria-Delbruck distributions, as described and performed in the Example 1. The replication error will depend on the nature of the microsatellite as well as on its length. For a given microsatellite length, mononucleotides will have a higher replication error rate than longer repeat units, and for a given repeat unit, replication error will increase as the number of repeat units increase.

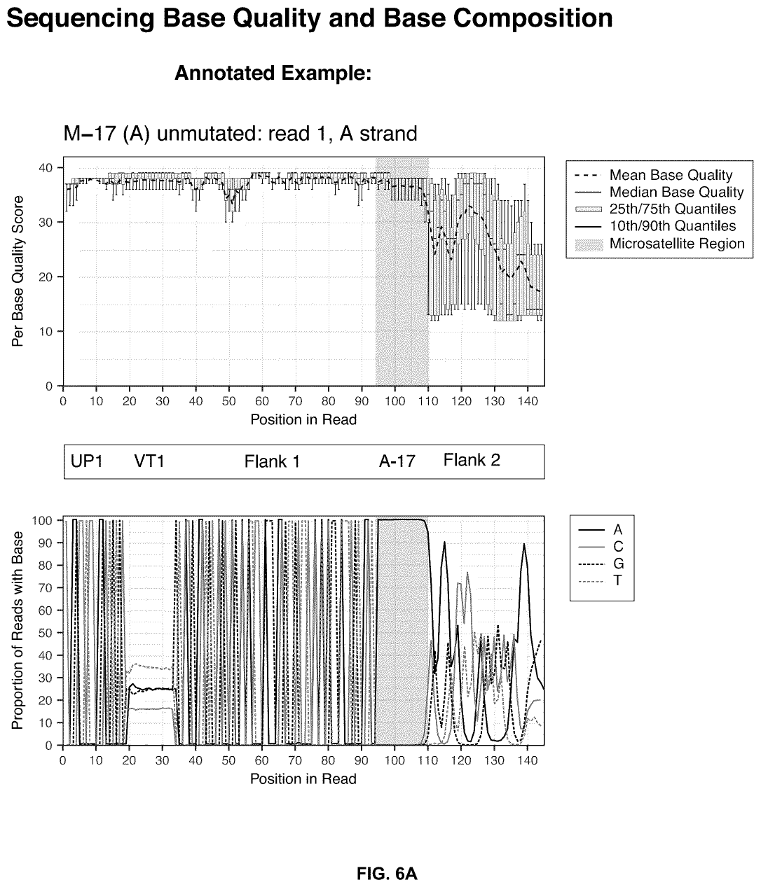

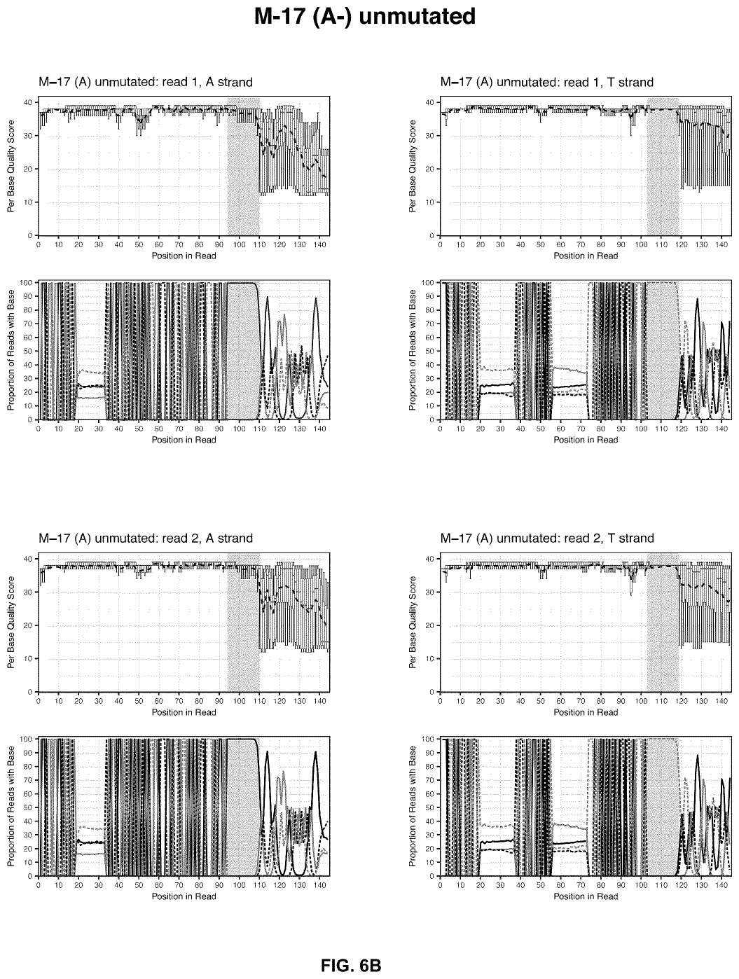

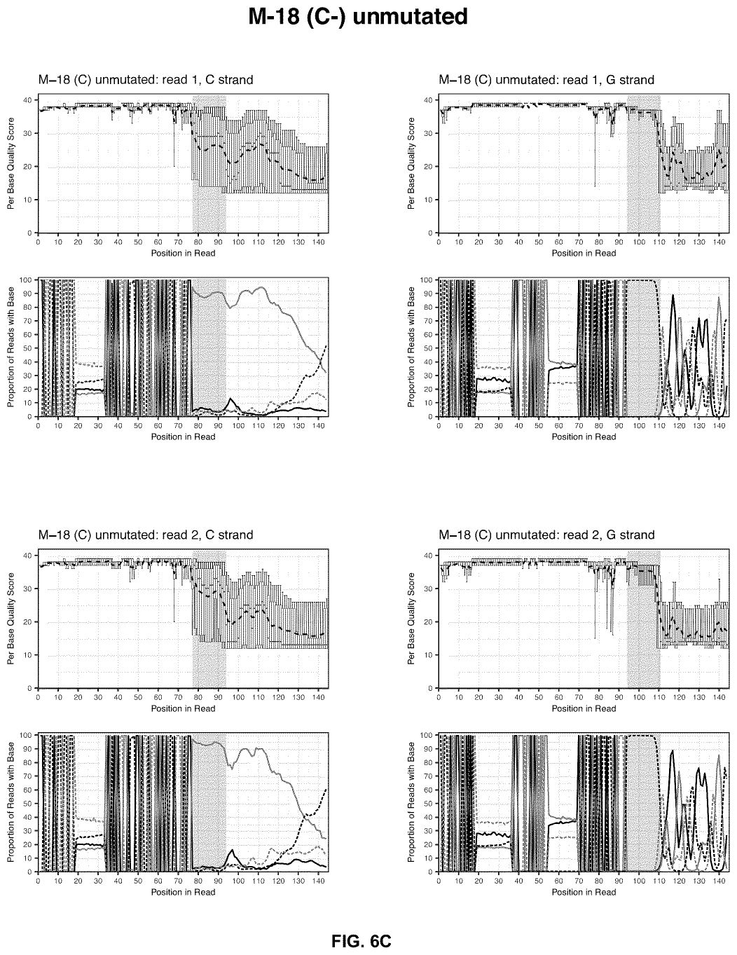

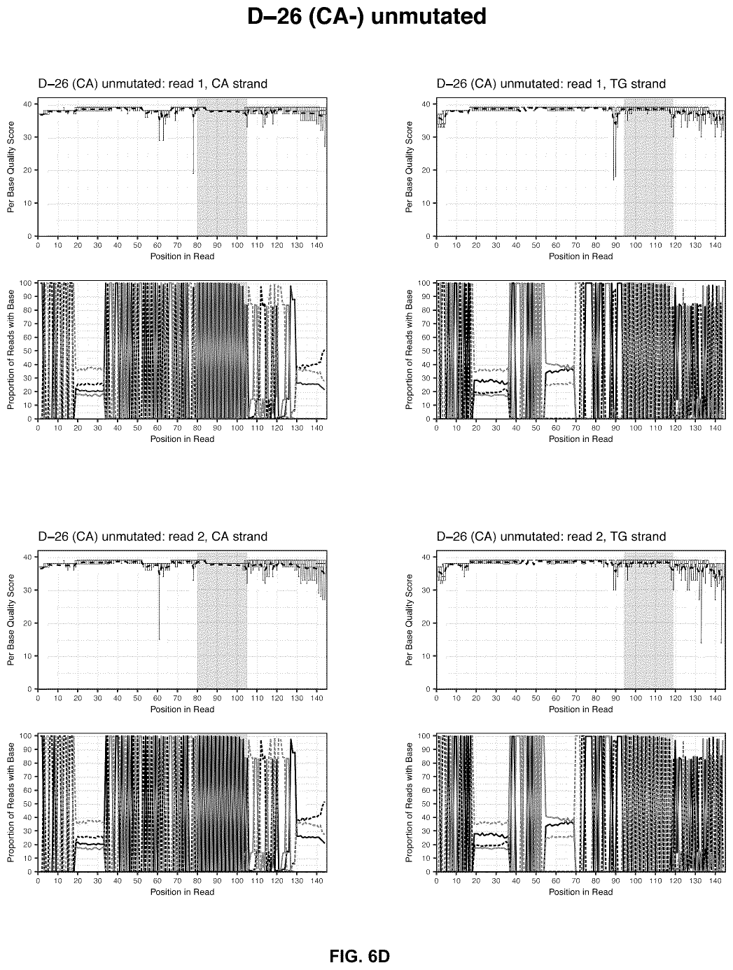

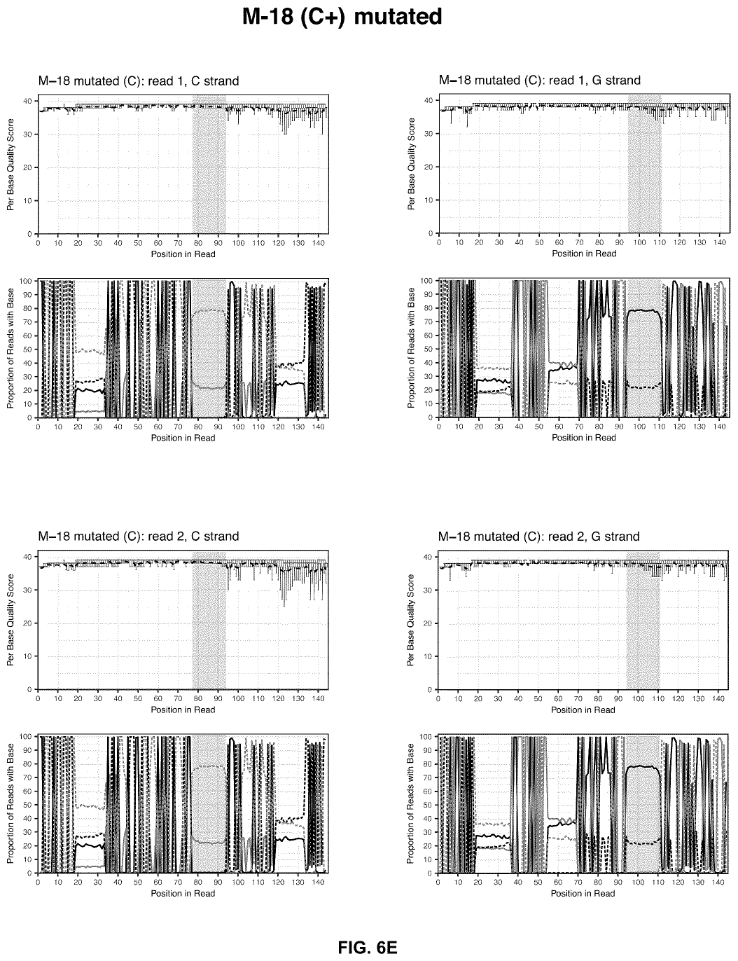

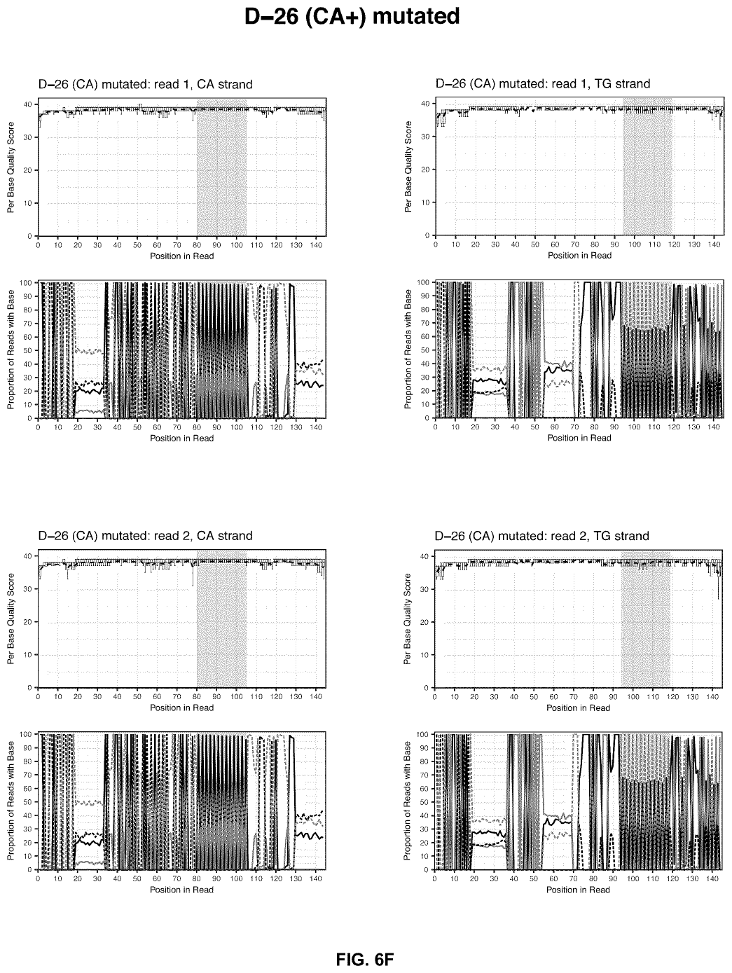

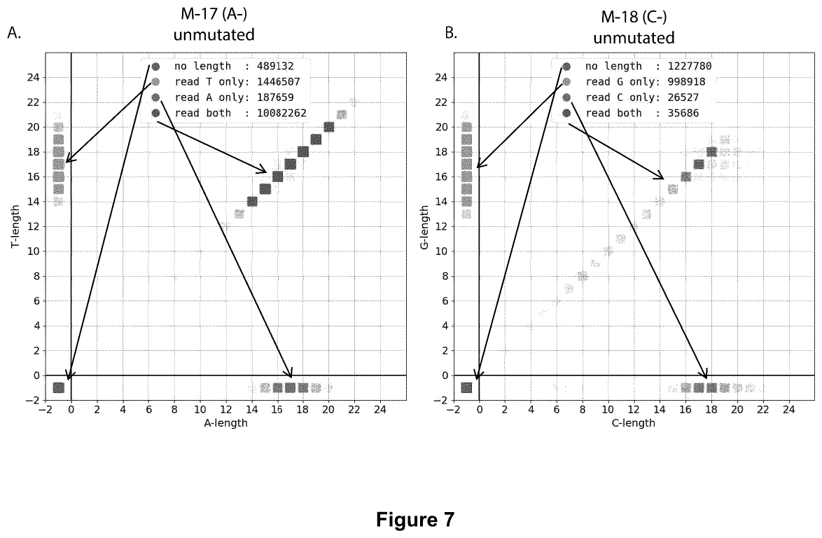

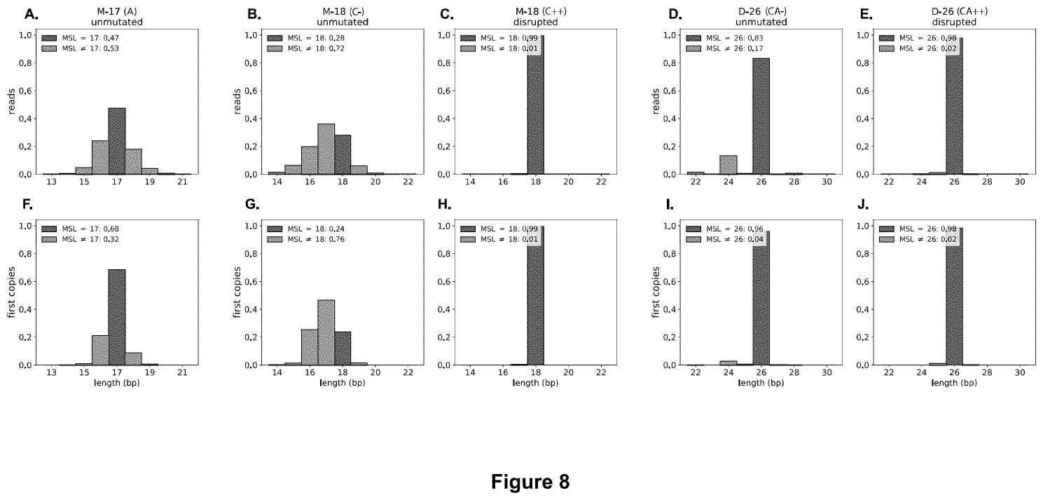

Sequence platform error: The measurement of repeats is further aggravated by the constraints of modern high-throughput sequencing platforms which are known to perform poorly with repeat sequences (Stoler, N. & Nekrutenko, A., 2021; Zavodna, M., et al., 2014; Nakamura, K. et al., 2011). Short-read sequencers (i.e. Illumina MiSeq, NextSeq, NovaSeq), which operate by sequencing-by-synthesis, require an initial amplification step on the sequencer. This amplification results in a cluster of molecules that are expected to contain identical sequences. However, due to replicative error discussed above, clusters generated from templates containing MS have many molecules that are out-of-phase with each other. This results in a high degree of uncertainty in the base calling algorithm during and after the repeat sequence, reflected in the per-base quality scores see in FIG. 6A to FIG. 6F of Example 1. When the base quality is sufficiently degraded by trying to read through a MS, the post-MS flank sequence is either not called or no longer resembles the actual sequence, which makes it impossible to determine the length of the MS in that read (see FIG. 7 of Example 1). Mononucleotide repeats are most affected by this phenomenon, but even dinucleotide repeat reads suffer from reduced sequencing quality scores in the post-MS flanks, particularly as they increase in length. Disruption of the microsatellite eliminates this machine error by reducing replicative error and thus avoiding the problems with clusters getting out of phase.

Previous methods for measuring microsatellite length: To tackle the problem of measuring microsatellite length, various approaches have been attempted to-date. Multiplex PCR and capillary electrophoresis methods have been described to measure 5-10 microsatellite loci (Bacher, J. W. et al., 2004; Boland, C. R. et al., 1998; Murphy, K. M. et al., 2006). MS lengths have been characterized from gene panels and high throughput sequencing data (Georgiadis, A. et al., 2019; Middha, S. et al., 2017), and statistical methods have been developed to increase accuracy in calling MS lengths from standard NGS data (Fungtammasan, A. et al., 2015; Highnam, G. et al., 2013). Finally, droplet digital PCR has been employed (Silveira, A. B. et al., 2020) to increase accuracy for small numbers of loci. None of these methods have the scale, depth, generality and accuracy needed for routinely monitoring a large set of microsatellite loci.

Partial mutagenesis: Mutagenesis is defined herein as a process applied to a template whereby copies of the template have a related but altered sequence. As used in the context of mutagenesis to aid in measurement of microsatellite lengths, one uses protocols that result in nucleotide substitutions in subsequent copies of the template, but preserve lengths.

Such length-preserving protocols can be achieved in either or both of two ways: first, modify the template so that its base pairing functionality is changed; second, copy the template under conditions such that nonstandard nucleotides are incorporated. In the first instance, one may use chemical modification, enzymatic treatment, nonstandard nucleotide substitutions, or combinations thereof. In each instance, the nonstandard nucleotides either have altered base pairing properties in and of themselves, or have altered base pairing properties following chemical or enzymatic treatment. After these steps are executed, copying the treated molecules using polymerases and standard nucleotides produce copies with altered sequences.

The desired mutagenic protocol useful for treatment of microsatellites is partial mutagenesis. If the mutagenesis is complete it may not destroy the repeat structure which makes replication error prone. With complete mutagenesis, the original repeat unit sequence may change to a new repeat sequence, but the number of repeat units may not change.

Any mutagenic process can be controlled. For example, the extent of chemical mutagenesis will depend on time, temperature, the concentration of reactants, pH and the like. If one is incorporating nonstandard nucleotides, the ratio of the nonstandard nucleotide to its standard counterpart (for examples, the ratio of methyl-cytosine to cytosine or inosine to adenine) will determine the extent of incorporation or replacement. The degree of mutagenesis achieved over each read can be determined from the substitutions observed in the flanking regions and in the microsatellite repeats themselves.

If mutagenesis is partial it will have these desirable properties:
(a) It will induce a “signature” nucleotide substitution in the final copies of the template that can distinguish the mutagenesis from random mutation that can occur by natural processes or polymerase error.
(b) Typically, when mutagenesis is partial, a variety of mutational patterns are created by the process. When this happens we can refer to “partial random mutagenesis.”

Partially mutagenized templates: As used herein “partially mutagenized templates” refers to any nucleic acid template that differs from an initial nucleic acid template as a result of partial mutagenesis. Therefore, an initial nucleic acid template that has been subjected to partial mutagenesis is a “partially mutagenized template.” Similarly, if a first copy of the partially mutagenized template is made, that is also referred to herein as a “partially mutagenized template.” Further, if a first copy of an initial nucleic acid template is made during a step of partial mutagenesis, that is also referred to herein as a “partially mutagenized template.” Still further, if a first copy of an initial nucleic acid template is made, and that first copy is then subjected to partial mutagenesis, that is also referred to herein as a “partially mutagenized template.”

Corresponding standard or nonstandard nucleotide: In the context of incorporating standard and nonstandard nucleotides into a template or a copy of a template, a standard nucleotide's “corresponding” nonstandard nucleotide refers to the nonstandard nucleotide that can be incorporated into a template or copy of a template instead of that standard nucleotide. Conversely, a nonstandard nucleotide's “corresponding” standard nucleotide refers to the standard nucleotide that can be incorporated into a template or copy of a template instead of that nonstandard nucleotide. For example, when incorporating standard and nonstandard nucleotides into a copy of a template by copying a template in the presence of a mixture of standard nucleotides and 5-methylcytosine, the corresponding standard nucleotide of 5-methylcytosine is cytosine because each can be incorporated into a copy of a template instead of the other.

Measuring length of MS in Sequencing reads: Microsatellites are tandem repeats, and have left and right flank sequences (flanking portions) that delineate them. This is illustrated in FIG. 1. The flank sequences are useful for two purposes:
(a) They map a read with a microsatellite to its locus;
(b) They delineate the boundaries of the microsatellite in the read.

Flank sequences: Flank sequences are also referred to herein as “flanking portions.” There is a flank length sufficient for purpose (a) above. This may be on the order of 12-15 bases when mapping a read to a population of templates enriched for microsatellite loci by panels, or on the order of 20-30 when mapping the locus to the entire genome. It is possible to map the read to its locus using only matches of the left or the right flank. Given that the read has been mapped to a specific locus with one flank, then a shorter length in the other flank may be sufficient for purpose (b), on the order of 3-5 bases to delineate the microsatellite-flank boundary (see FIG. 2). The flank sequences in a read are subjected to mutagenesis along with the microsatellite sequence. Thus, when identifying and mapping the flank sequences, both the reference flank search sequences and the read sequences can be fully converted to aid the mapping and alignment process. Once the read has been assigned to its locus and the boundaries of the microsatellite are identified using the flank sequences (flanking portions), the microsatellite length (MSL) can be determined from the difference in the position of the two flank boundary positions.

Distributions of Microsatellite Lengths: Over any population of templates, there will be a distribution of microsatellite lengths, the initial microsatellite length distribution, reflecting the replicative history of the cells or organisms from which those templates were drawn. Upon sequencing the reads from copies of the mutagenized templates, one obtains instead a distribution of microsatellite lengths, which, if the reads are selected to have a disrupted microsatellite structure, will accurately reflect that initial distribution.

In practice, one makes distributions over sets of reads where the reads are partitioned into sets by the locus of their provenance, as identified by flanking sequences. For each such set, one can make further subsets of reads further partitioned by first copies or templates of origin, or both, provided sequenced reads include identifiers, such as varietal tags or partial mutagenesis patterns, to make these assignments. For any distribution over a set of reads, one can apply a consensus rule to get a consensus length. One then can obtain thereby distributions of consensus lengths. For example, from the consensus over first copies, one obtains a distribution of first copy consensus lengths over initial templates. Furthermore, one can take a consensus from the distribution of first copy consensus lengths to obtain a consensus length for each initial template. These are all steps one makes to more accurately assess the initial microsatellite length distribution.

A consensus rule is a function that takes as input a microsatellite length distribution and returns a single value if possible. Examples of consensus rules include:
(a) Unanimous rule: L if the distribution has a single length L.
(b) Plurality rule: L if L is the most common length in the distribution.
(c) Majority p-rule: L if there is a length L in the distribution with a frequency greater than p where p is >=0.5.

For data with multiple loci, each locus has a unique microsatellite length distribution. This collection of microsatellite length distributions determines the microsatellite length distribution profile for the sample.

Individual identifiers or individual tags: Initial templates and/or first copies are tagged in a way such that individual templates can be distinguished, and subsequent copies retain information from the tags. Without limitation, the individual tags, also referred to herein as “individual identifiers”, may be any combination of: (1) randomized nucleotide sequences added to the ends of templates by extension or ligation (i.e., varietal tags); (2) diverse fragment end positions created during extraction from biological specimens, following physical fragmentation, or following treatment with non-specific endonucleases; or (3) a pattern of partial mutation, as introduced into the initial template in a step of partial mutagenesis. As a result of this tagging, two or more copies derived from the same initial template can be so identified.

Varietal tags: Varietal tags are described in U.S. Pat. No. 9,404,156, the entire contents of which is specifically incorporated herein by reference. As discussed in U.S. Pat. No. 9,404,156, varietal tags comprise nucleotide sequences that are sufficiently unique with respect to one another such that the combination of a varietal tag with a nucleic acid molecule from the input sample (e.g. initial nucleic acid templates) provides an essentially unique tagged nucleic acid molecule that can only with very low probability be replicated by chance from another like molecule (a chance of roughly 1/N where N is the number of available tags). Accordingly, within a pool of tagged nucleic acid molecules, each tagged nucleic acid molecule is likely unique in the pool when a sufficiently large number of distinct tags is used. Without limitation, varietal tags of the invention may be 4, 5, 6, 7, 8, 9, 10, 11, 12, 13, 14, 15 up to 1000 nucleotides long. A person skilled in the art can readily determine the number of distinct tags that should be used for a given sample to ensure that each tagged nucleic acid molecule is likely unique in the pool. Varietal tags may also comprise sample tags. Sample tags are similar to varietal tags except that the same sample tag is attached to each member of a unique sample in order to identify the nucleic acid molecule as a member of that unique sample. Using sample tags, multiple unique samples can be pooled and processed simultaneously according to the methods described herein because the sequence reads of nucleic acids originating from the same unique sample can be identified and deconvoluted. Varietal tags may also comprise universal primer sequences which, in conjunction with a universal primer, may be used to amplify tagged nucleic acid molecules. In an embodiment, the set of tags are sufficiently large, such that tagged nucleic acid molecules differ at more than one nucleotide, and so tagged nucleic acid molecules that differ by a single nucleotide are determined to originate from the same initial template. In an embodiment, the varietal tags lack nucleotides that are susceptible to the step of partial mutagenesis used in the method.

First copies: Copies of nucleic acid molecules produced directly from a template nucleic acid molecule. First copies may be produced from a template nucleic acid molecule by linear amplification.

Linear amplification: A method which amplifies a nucleic acid molecule by producing copies only from an original nucleic acid molecule and not from its copies. One method of linear amplification involves a polymerase chain reaction (PCR) in the presence of only a forward primer or only a reverse primer, such that only the original nucleic acid molecule (and not its copies) are used as the template from which additional copies of the nucleic acid molecule are produced.

Standard nucleotides: In the context of amplification or coyping a nucleic acid templates, standard nucleotides refers to the nucleotides present in a standard polymerase chain reaction (PCR) nucleotide mixture, i.e. deoxyadenosine triphosphate (dATP), deoxycytidine triphosphate (dCTP), deoxyguanosine triphosphate (dGTP), and deoxythymidine triphosphate (dTTP).

Purification moiety: A purification moiety refers to a chemical moiety which has a specific binding affinity with another molecule and may be used in affinity purification. One example of a purification moiety is biotin, which has a high specific binding affinity to streptavidin.

### Sequence Read Analysis

Proper read: A proper read pair has a good match (up to one mismatch) to each of the UP1, UP2, and UP3 regions (if used in the protocol) and the proximal flank of the microsatellite in both reads. A proper read always has a proximal flank, but it is possible that the distal flank could not be clearly identified. In those cases, the read does not report a length.

Qualified: A read pair is considered qualified if both paired-end reads agree on the MSL, or if only one read of the pair reports a length. These MSL are then labeled as on-target if they are the expected length (i.e. 17, 18, or 26), and off-target, otherwise.

Disruption indices: For qualified reads, the degree to which the microsatellite is disrupted by the mutagenesis is measured in two ways. The first is the MS conversion rate, for example the proportion of C bases converted to T in the MS, for a bisulfite mutagenesis protocol. The second is the maximum repeat length, which is the largest number of tandem repeat units remaining in the microsatellite.

Disrupted: A read is defined as disrupted if the MS conversion rate is between 0.15 and 0.85 and the maximum repeat length does not exceed a defined threshold. In embodiments, this defined threshold is five. In other embodiments, this threshold is six or seven. A first copy is labeled disrupted if the median disruption parameters over its qualified reads are within the bounds to call a read disrupted.

Properly-covered: A first copy is called properly-covered if it has a sufficient number of proper reads. The threshold for the number of reads required to define properly-covered depends on the complexity and depth of the library.

Well-covered: A first-copy is called well-covered if it is properly-covered and has at least three qualified reads.

Modal length: The modal length of a first copy is defined as the value most commonly seen among all the reads associated with it.

### General

All combinations of the various elements and embodiments disclosed herein are within the scope of the invention.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

This invention will be better understood by reference to the Examples which follow, but those skilled in the art will readily appreciate that the specific experiments detailed are only illustrative of the invention as described more fully in the claims which follow thereafter.

### EXAMPLES

In the examples that follow, the problem of accurate measurement of MSLV is solved by using partial mutagenesis to disrupt enough of the repeat structure so that templates containing microsatellites can replicate faithfully, yet not so much that the flanking regions cannot be reliably identified. Compared to untreated templates, the methods described herein achieve three orders of magnitude reduction in the rate of error per round of replication. By requiring two independent first copies of an initial template, the methods described herein reach error rates below one in a million.

### Example 1—Accurate Measurement of Microsatellite Length Following Disruptive Mutation on Synthetic Templates

The following example demonstrates the microsatellite length determination of synthetic templates. It illustrates many of the principles of the method described herein: the high level of sequence error for unmutated microsatellites, especially mononucleotide tracts; high levels of platform sequence error over unmutated mononucleotide tracts; partial bisulfite mutagenesis; estimating replicative error; the relationship between accuracy of length measurement and the degree of microsatellite disruption; labeling initial templates and first copies for later identification; improvement in accuracy by aggregating length measurements over many reads, first copies and initial templates; and how to compute error over small numbers of reads distributed over templates and first copies.

### Example 1a: Template Design

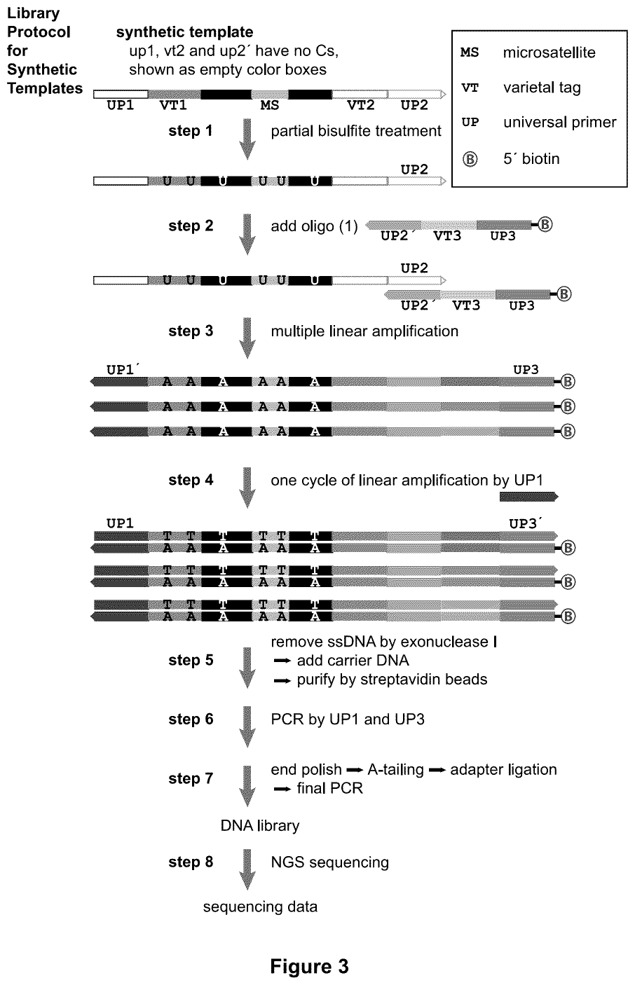

For the testing and development of this method, three synthetic templates containing microsatellite (MS) tracts were used. The MS sequences are: a 17 base-pair mononucleotide A repeat called M-17 (A), an 18 base-pair mononucleotide C repeat called M-18 (C), and a 26 base-pair dinucleotide CA repeat called D-26 (CA). The templates were ordered from Integrated DNA Technologies (IDT). The full sequences of the synthetic templates, oligonucleotide adaptors, and primers are given in Table 1. As shown in FIG. 3, the structure of synthetic templates used for the partial mutagenesis protocol, M-18 (C) and D-26 (CA), is as follows: a 5′ primer binding site without cytosine (UP1), a 15-mer varietal tag sequence (VT1) with random nucleotides represented as “NNN . . . ”, a 5′ flanking sequence, a C or CA microsatellite, another 3′ flanking sequence, another 15-mer varietal tag (VT2) with random nucleotides represented as “DDD . . . ”, and finally a 3′ binding site (UP2) without cytosine. Templates containing mononucleotide A, denoted as M-17 (A), which did not undergo mutagenesis were also examined. These had a very similar design to the C microsatellite templates detailed in Table 1. The notation M-18 (C+) and D-26 (CA+) is used to denote the templates and libraries after mutagenesis, and M-18 (C−), D-26 (CA−), and M-17 (A−) are used to refer to libraries without mutagenesis.

TABLE 1
Sequence information of the templates and the oligonucleotides
The sequences for the 3 template sequences: M-18 (C), D-26 (CA), and M-17 (A) are
listed. N's indicate random nucleotides; D's indicate random nucleotides excluding
C. Oligo (1) and primer sequences UP1 and UP3 are also listed for the two protocols used.
A. For the templates M-18 (C) and D-26 (CA)

| Template | Sequence (5′ to 3′) |
|---|---|
| M-18 (C) | AAGTTGGATGGTGGTGGTNNNNNNNNNNNNNNNCACGACATTCCAGGGAACGTTGCT |
| | TCACATATGGATTTCGTAACCCCCCCCCCCCCCCCCCGAAAGTACCTCCTATCGTCC |
| | GTATDDDDDDDDDDDDDDDGGAGAGTGTGGAGGGTTG (SEQ ID NO: 3781) |
| D-26 (CA) | AAGTTGGATGGTGGTGGTNNNNNNNNNNNNNNNAGCCTAATACTACAGTGCCGAGCG |
| | CACGCGTGCCTGCTGACATTGTCACACACACACACACACACACACACATTTCCTGGG |
| | CTTGTATTTTCCAAADDDDDDDDDDDDDDDGGAGAGTGTGGAGGGTTG (SEQ ID |
| | NO: 3782) |
| Oligonucleotide | Sequence (5′ to 3′) |
| oligo (1) | /5Biosg/GTGGTATCAACGCAGAGTNNNNNNNNNNNNNNNNNNCAACCCTCCACAC |
| | TCTCC (SEQ ID NO: 3783) |
| UP1 | AAGTTGGATGGTGGTGGT (SEQ ID NO: 3784) |
| UP3 | GTGGTATCAACGCAGAGT (SEQ ID NO: 3785) |

B. For the template M-17 (A)

| Template | Sequence (5′ to 3′) |
|---|---|
| M-17 (A) | CAAGCCTCCAAGCTGTGCNNNNNNNNNNNNNNNGTACCGAACTGTACGCACGACATT |
| | CCAGGGAACGTTGCTTCACATATGGATTTCGTAACTAAAAAAAAAAAAAAAAACGAA |
| | AGTACCTCCTATCGTCGTCGACAAANNNNNNNNNNNNNNNNNNACTCTGCGTTGATA |
| | CCAC (SEQ ID NO: 3786) |
| Oligonucleotide | Sequence (5′ to 3′) |
| oligo (1) | /5Biosg/AAAGTTGCATGGTGCTGGTNNNNNNNNNNNNNNNNNNGTGGTATCAACG |
| | CAGAGT (SEQ ID NO: 3787) |
| UP1 | CAAGCCTCCAAGCTGTGC (SEQ ID NO: 3788) |
| UP3 | AAAGTTGCATGGTGCTGGT (SEQ ID NO: 3789) |

### Example 1B: Protocol for Partial Mutagenesis, Library Preparation, and Sequencing

An operational protocol for partial mutagenesis of microsatellite templates is described here and in FIG. 3. In step 1 of the mutagenesis protocol, 80 ng of each of the two templates containing C, M-18 (C) and D-26 (CA), was partially bisulfite converted (or not) by EZ DNA Methylation-Direct Kit (Zymo Research). Incubation time and temperature for bisulfite conversion were chosen to approach an ideal bisulfite conversion rate of close to 50%. In this protocol, DNA was incubated at 55° C. for 40-50 min. In fact, this protocol achieved 77% and 66% conversion for the M-18 (C+) tract and the D-26 (CA+) tract, respectively.

After conversion, about 6×10⁴ original templates underwent 9 cycles of linear amplification (steps 2 and 3) using a biotinylated oligo. This produced first round copies (first copies, for short) with a structure that had a 5′ biotinylated UP3 and a VT3 represented as “NNN . . . ” in the Table 1 and in gray scale in FIG. 3. Double-stranded DNA fragments were obtained in another round of linear amplification by using UP1 (step 4). In step 5 extra free oligo was removed by Thermolabile Exonuclease I (NEB). After adding 50 ng of carrier DNA (poly (A), Sigma-Aldrich), biotinylated DNA fragments were purified by streptavidin beads (NEB). In step 6, 18 cycles of the exponential PCR were carried out using UP1 and UP3 to generate enough material to prepare the DNA libraries for sequencing. Standard steps for library preparation (end polishing, A-tailing, adapter ligation) were utilized to complete the sequencing library preparation (step 7). All libraries were prepared with variable length library barcodes (Shi, J. et al., 2015), and then pooled. The pooled libraries were sequenced as 2×150 bp paired-end runs on an Illumina MiSeq™ (step 8).

In steps 3 and 4 (linear amplification of first copies) NEBNext® Q5U® Master Mix was used. This master mix contains modified Q5® High Fidelity DNA Polymerase, optimized for amplification of uracil-containing templates. In step 6 Phusion Flash High-Fidelity PCR Master Mix (Thermo Fisher Scientific) was used for 18 cycles of PCR. This master mix contains Phusion Flash II DNA Polymerase which has high-fidelity and is excellent for multiplex PCR. In step 7, for library preparation, NEBNext® Ultra™ II Q5® Master Mix (NEB) was used. This master mix contains Q5® High Fidelity DNA Polymerase, optimized for amplification of NGS libraries.

The parallel protocol without bisulfite treatment, used for the unmutated C templates, M-18 (C) and D-26 (CA) and M-17 (A), had the following differences: the number of original templates was about 3×10⁴, and in step 6, 14 or 15 cycles of PCR were employed.

### Example 1C: Sequence Processing and Tabulation

All read pairs were first evaluated for having the proper structure. A proper read pair has a good match (up to one mismatch) to each of the UP1, UP2, and UP3 regions and the proximal flank of the microsatellite in both reads. From a proper read pair, it is possible to extract the three varietal tags which identify the template (VT1, VT2) and first copy (VT3). From each read of the pair, a search was also conducted for a good match to the distal flank sequence (up to one mismatch), and if the distal flank was found, a microsatellite length (MSL) was reported based on the distance in base pairs (bp) between the flanks within the read. A proper read always has a proximal flank, but it is possible that the distal flank could not be clearly identified. In those cases, the read did not report a length. A read pair is considered qualified if both paired-end reads agree on the MSL, or if only one read of the pair reports a length. These MSL are then labeled as on-target if they are the expected length (i.e. 17, 18, or 26), and off-target, otherwise.

For qualified reads, the degree to which the microsatellite is disrupted by the mutagenesis is measured in two ways (disruption indices). The first is the MS conversion rate or the proportion of C bases converted to T in the MS. The second is the maximum repeat length, which is the largest number of tandem repeat units remaining in the microsatellite. A read is defined as disrupted if the MS conversion rate is between 0.15 and 0.85 and the maximum repeat length does not exceed five. See Table 2 for the expected yield of microsatellite disruption as a function of the average bisulfite conversion rate, as determined by simulation. The observed levels of disruption follow closely the expectations from simulations.

TABLE 2
Disruption yields as a function of mutation rate
For the M-18 (C+) and D-26 (CA+) templates, the proportion of reads passing each
of the two disruption parameter thresholds is modeled as a function of overall mutation rate.
The reads that pass the rate cutoff are the reads with between 15% to 85% of C's mutated,
and the reads that pass the repeat length cutoff have 5 or fewer units of the repeat intact.
Highlighted in bold are the mutation rate bins that most closely match the observed data.

| M-18 (C+) | | | | D-26 (CA+) | | | |
|---|---|---|---|---|---|---|---|
| | pass | pass | | | pass | pass | |
| | conversion | repeat | | | conversion | repeat | |
| | rate | length | | | rate | length | |
| mutation | thresholds | threshold | | mutation | thresholds | threshold | |
| rate | (15-85%) | (<=5) | pass both | rate | (15-85%) | (<=5) | pass both |
| 0.05 | 0.05936 | 0.00622 | 0.00622 | 0.05 | 0.1356 | 0.03421 | 0.03421 |
| 0.1 | 0.26615 | 0.04262 | 0.04262 | 0.1 | 0.38117 | 0.12517 | 0.12517 |
| 0.15 | 0.51932 | 0.12061 | 0.12061 | 0.15 | 0.60058 | 0.24773 | 0.24773 |
| 0.2 | 0.72837 | 0.2326 | 0.2326 | 0.2 | 0.76585 | 0.38283 | 0.38283 |
| 0.25 | 0.86473 | 0.36285 | 0.36285 | 0.25 | 0.8742 | 0.51469 | 0.51469 |
| 0.3 | 0.93828 | 0.49772 | 0.49772 | 0.3 | 0.93599 | 0.63861 | 0.63861 |
| 0.35 | 0.97632 | 0.61845 | 0.61845 | 0.35 | 0.97089 | 0.73097 | 0.73097 |
| 0.4 | 0.99163 | 0.70939 | 0.70939 | 0.4 | 0.9873 | 0.80323 | 0.80323 |
| 0.45 | 0.99737 | 0.77244 | 0.77244 | 0.45 | 0.99455 | 0.84632 | 0.84632 |
| 0.5 | 0.99879 | 0.79238 | 0.79238 | 0.5 | 0.99659 | 0.86161 | 0.86161 |
| 0.55 | 0.99728 | 0.76924 | 0.76924 | 0.55 | 0.99435 | 0.848 | 0.848 |
| 0.6 | 0.99224 | 0.71046 | 0.71046 | 0.6 | 0.98754 | 0.80341 | 0.80341 |
| 0.65 | 0.97657 | 0.61781 | 0.61781 | 0.65 | 0.9702 | 0.73203 | 0.73203 |
| 0.7 | 0.93949 | 0.49742 | 0.49742 | 0.7 | 0.93576 | 0.63515 | 0.63515 |
| 0.75 | 0.86632 | 0.36552 | 0.36552 | 0.75 | 0.87283 | 0.51461 | 0.51461 |
| 0.8 | 0.72752 | 0.23116 | 0.23116 | 0.8 | 0.76624 | 0.38251 | 0.38251 |
| 0.85 | 0.51784 | 0.11924 | 0.11924 | 0.85 | 0.60403 | 0.24738 | 0.24738 |
| 0.9 | 0.26888 | 0.04391 | 0.04391 | 0.9 | 0.38108 | 0.12797 | 0.12797 |
| 0.95 | 0.05703 | 0.00595 | 0.00595 | 0.95 | 0.1354 | 0.03507 | 0.03507 |

A set of 5 tables were created from the proper reads for each of the 5 libraries (3 templates and 2 protocols): M-17 (A−), M-18 (C−), D-26 (CA−), M-18 (C+), D-26 (CA+), which are called the READ TABLE. The READ TABLE records the varietal tag information for the read, the MSL if the read is qualified (−1, otherwise), and its two disruption indices.

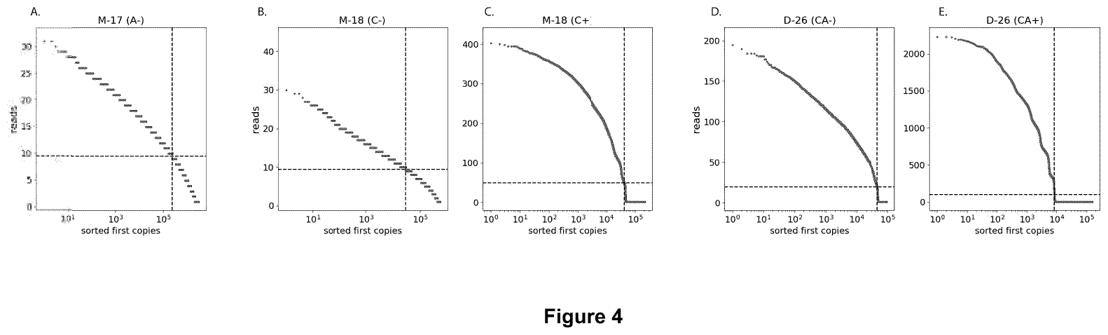

From the READ TABLES, the FIRST COPY TABLES were then created. A first copy is marked by its template tag-pair (VT1, VT2) and its first copy tag (VT3). A first copy is called properly-covered if it has a sufficient number of proper reads. The threshold for the number of reads required to define properly-covered depends on the complexity and depth of the library and was 10, 10, 20, 50, and 100 for M-17 (A−), M-18 (C−), D-26 (CA−), M-18 (C+), D-26 (CA+), respectively. The choice for these cutoffs, given the actual distribution of available reads, are shown in FIG. 4. A first-copy is called well-covered if it is properly-covered and has at least three qualified reads. For each well-covered first copy, the MSL was counted over all qualified reads to determine the modal length, which is the most common length reported by all reads associated with that first copy. A first copy is labeled disrupted if the median disruption parameters over its qualified reads are within the bounds to call a read disrupted.

For each first copy, the number of proper reads, the number of qualified reads, the modal length, and the number of qualified reads that report the modal length were all tabulated. For each first copy, the median disruption indices of its qualified reads were also recorded, where applicable. A WELL-COVERED FIRST COPY TABLE was then created by restricting to rows with a sufficient number of proper and qualified reads. This filtering step eliminated VT combinations with low read coverage that result from single-base errors in the varietal tag sequences.

From the WELL-COVERED FIRST COPY TABLE, a TEMPLATE TABLE was generated. A template is any template tag-pair (VT1, VT2) with at least one well-covered first copy. For each template, the number of qualified reads, the number of well-covered first copies, and the median disruption indices from its first copies were all counted. If those median disruption indices fall within the criteria defined for a disrupted read, the template is called disrupted. The modal lengths of the well-covered first copies for each template was also recorded. A template is called well-covered if it has at least three well-covered first copies. Well-covered templates are flagged as synthetic variants if three or more first copies unanimously agree on an MSL length different from expected.

### Example 1D: Modeling Error

Two methods are used for modeling error. The first uses the method of moments; the second uses Luria-Delbruck Diffusions, or LDD (Luria, S. E. & Delbrück, 1943). The first method utilizes the varietal tags to identify reads with the first copies and the template from which they arose. Even if all templates are of the same initial length, the first copies may have different lengths due to replication error. Moreover, each first copy has its own probability of error due to random events occurring during exponential growth. The N-th moment for length L in the read data is defined as the mean probability that N reads from each chosen first copy are unanimous for length L. Thus read error is nearly identical to the 1-st moment for any given L.

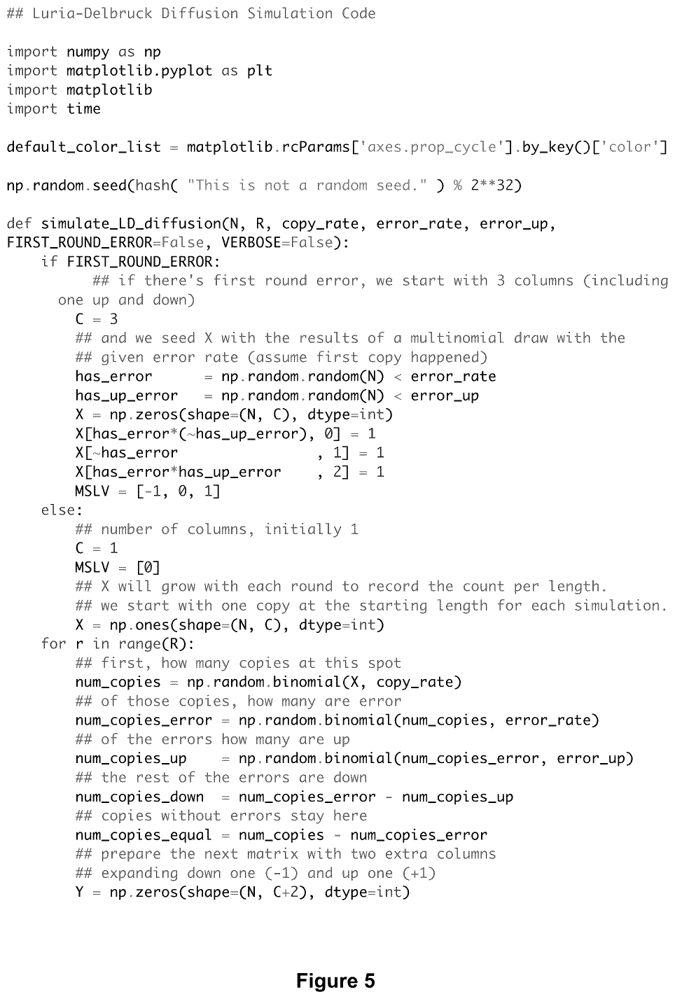

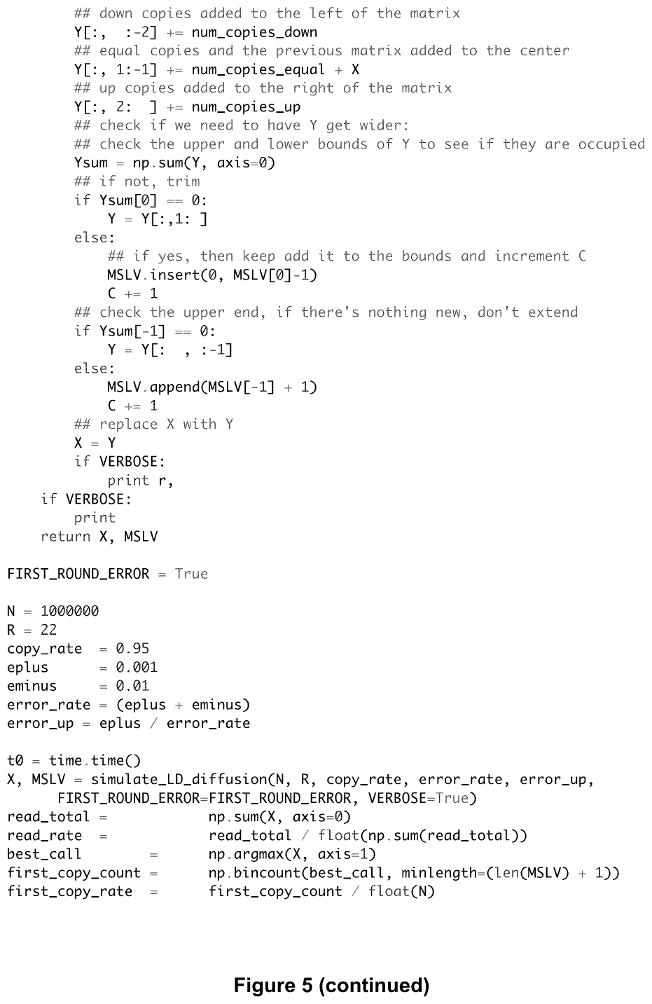

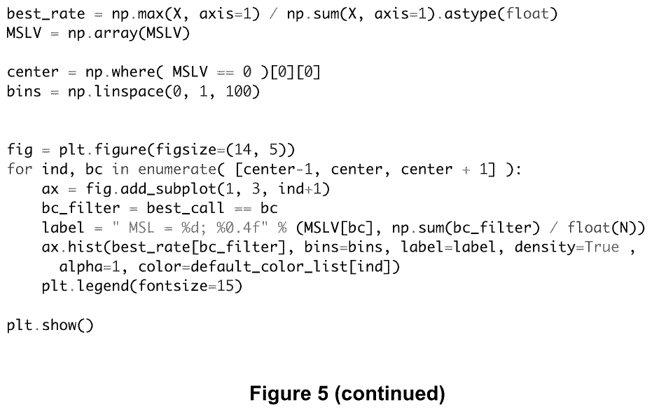

In the second method, per-round PCR error rates are estimated with LDD. In a simple LDD, each round of an exponential amplification is modeled such that each single stranded nucleic acid of length L replicates with efficiency e, and its copy then retains its length, or decreases or increases its length by one unit according to two errors rate parameters: α and β. For simplicity it is assumed that α and β are not functions of strand or L, and the length increases or decreases only by the length of a single tandem repeat unit. All copies are retained in each round, but to simulate the protocol, the original template is not retained. After seeding with a single original template and R rounds of replication, a simulated distribution of MS lengths is obtained. α and β are estimated for the templates by simulating LDD distributions over a grid of parameter values and identifying the best match to the observed read error rate (see FIG. 5 for algorithm details).

### Example 1E: Experimental Design

The performance of the partial mutagenesis protocol for microsatellites are evaluated by examining 5 sequencing libraries generated from 3 synthetic templates using 2 protocols.

As shown in the top of FIG. 3, the templates contain mononucleotide A, mononucleotide C, and dinucleotide CA microsatellites (MS), flanked on either side by common sequence (black). 5′ and 3′ to the common sequence are randomly generated varietal tag sequences (VT1 and VT2) that uniquely label each template molecule. Flanking the varietal tags are universal primer sequences (UP1 and UP2′) designed without C nucleotides if needed to resist bisulfite mutagenesis.

FIG. 3 shows a protocol for bisulfite mutagenesis, followed by library preparation and sequencing. The protocol is discussed in more detail in the Example 1B. Briefly, the templates were either bisulfite treated or not, in step 1. A biotinylated primer complementary to the 3′ universal primer UP2′, with its own unique varietal tag (VT3, gray) and a universal primer sequence (UP3) is added in step 2. Multiple first copies are generated (step 3), each with the same VT1-VT2 and a unique first copy tag VT3. The first copies are made double-stranded (step 4), purified by streptavidin chromatography (step 5), and amplified by PCR (step 6). Finally, the PCR products are made into libraries with specific barcodes, pooled and sequenced to high depth (steps 7-8).

The 5 sequencing libraries are named M-17 (A−), M-18 (C−), D-26 (CA−), M-18 (C+), D-26 (CA+), corresponding to M (mono-) or D (di-nucleotide), their microsatellite length, the sequence of the microsatellite repeat unit, and whether mutagenesis was applied (+/−). These are abbreviated to A−, C−, CA−, C+ and CA+, respectively. A template is disrupted if the mutagenesis resulted in sufficient conversion to corrupt the repeat structure (see Materials and Methods). In the analyses below, when the data for the mutated libraries is restricted to only the disrupted templates, these datasets are referred to as M-18 (C++) or C++, and D-26 (CA++) or CA++. The proportion of disrupted reads in the C++ and CA++ libraries is 29% and 73%, respectively, which falls close to the expected proportion, given the conversion rates (see Table 2).

Below the properties of the microsatellite lengths observed in the data for A−, C−, CA−, C++, and CA++, are described at three levels of organization: (1) reads, (2) first copies, and (3) templates. At the top level are the templates which refer to the original synthesized molecules. These are uniquely identified by their VT1-VT2 tag-pair. The next level is the first copies which are generated during the first round of linear amplification (FIG. 3, step 2). First copies are identified by the unique triplet: the VT1-VT2 pair from their initial template, and the unique VT3 added to the molecule during linear amplification. At the bottom level are the reads of the sequencing library. For those reads with the correct structure, its three varietal tags and, when possible, the length of the microsatellite are determined. For details on data processing of reads, first copies and templates and for definitions of the terms used below, see Example 1C.

### Example 1F: Estimates of MSL Error Rates from Data

### Reads

Within a sequencing read, measuring the MSL depends on identifying the expected proximal and distal flank sequences and measuring their distance in the read. When parsing the unmutated mononucleotide reads, in particular the M-18 (C−) library, it was discovered that the base quality of the read decays considerably after reading through the microsatellite sequence (FIG. 6A to FIG. 6F). In many cases, this decay of base quality is so bad that the distal flank sequence could not be identified in the read. In the M-18 (C−) dataset, only 46% of proper reads are qualified, and of those 94% failed to report a length from the C-repeat read. In contrast, for the M-17 (A−) 95% of proper reads are qualified, and for the remaining sets, 99% of reads are qualified (FIG. 7).

In FIG. 8, panels A-E, the microsatellite length determinations per read are shown as a histogram for each of the five libraries. In the histogram, qualified reads that match the expected length are shown in darker grey (on-target), while those reporting a different length are shown in lighter grey (off-target). In general, off-target lengths tend to be shorter rather than longer. Read counts for each MS length and library are shown in Table 3, part A.

TABLE 3
MS length distributions in reads and first copies
For each of the five libraries, this table shows the number of reads and number of first copies that show each possible
microsatellite length, from <=−5 to >=+5 from the expected length. For the three most stable libraries, where it was possible
to identify and remove synthetic variants, the resulting counts after removing synthetic variants are shown.

| | | | MSLV deviation from expected length | | | | | | | | | | |
|---|---|---|---|---|---|---|---|---|---|---|---|---|---|
| A. READS | | | <=−5 | −4 | −3 | −2 | −1 | 0 | +1 | +2 | +3 | +4 | >=+5 |
| M-17 (A−) | unmutated | all | 1540 | 2382 | 15687 | 129032 | 662810 | 1318654 | 496859 | 114349 | 19831 | 3342 | 5390 |
| M-18 (C−) | unmutated | all | 4913 | 2511 | 10068 | 31175 | 56529 | 44531 | 9785 | 1360 | 229 | 51 | 200 |
| M-18 (C++) | disrupted | all | 1072 | 1 | 5 | 14 | 8451 | 1641719 | 60 | 2 | 0 | 0 | 0 |
| D-26 (CA−) | unmutated | all | 8957 | 37395 | 2357 | 339586 | 14455 | 2120035 | 5291 | 18719 | 18 | 702 | 121 |
| D-26 (CA++) | disrupted | all | 8395 | 206 | 848 | 2544 | 51425 | 4413601 | 14353 | 244 | 1 | 10 | 3 |
| M-18 (C++) | disrupted | wc - synth | 2 | 1 | 5 | 12 | 50 | 1612051 | 54 | 2 | 0 | 0 | 0 |
| D-26 (CA−) | unmutated | wc - synth | 3777 | 32367 | 413 | 303560 | 1321 | 1910332 | 450 | 16818 | 0 | 589 | 115 |
| D-26 (CA++) | disrupted | wc - synth | 61 | 196 | 2 | 2453 | 2076 | 4312537 | 405 | 236 | 0 | 10 | 3 |

| | | | MSLV deviation from expected length | | | | | | | | | | |
|---|---|---|---|---|---|---|---|---|---|---|---|---|---|
| B. FIRST COPIES | | | <=−5 | −4 | −3 | −2 | −1 | 0 | +1 | +2 | +3 | +4 | >=+5 |
| M-17 (A−) | unmutated | all | 96 | 58 | 180 | 2250 | 50762 | 164225 | 20588 | 1515 | 112 | 13 | 5 |
| M-18 (C−) | unmutated | all | 294 | 81 | 383 | 7171 | 13146 | 6717 | 428 | 11 | 0 | 0 | 4 |
| M-18 (C++) | disrupted | all | 7 | 0 | 0 | 0 | 58 | 11644 | 0 | 0 | 0 | 0 | 0 |
| D-26 (CA−) | unmutated | all | 86 | 82 | 23 | 1211 | 275 | 44142 | 95 | 36 | 0 | 2 | 1 |
| D-26 (CA++) | disrupted | all | 13 | 0 | 2 | 0 | 61 | 5852 | 18 | 0 | 0 | 0 | 0 |
| M-18 (C++) | disrupted | wc - synth | 0 | 0 | 0 | 0 | 0 | 11415 | 0 | 0 | 0 | 0 | 0 |
| D-26 (CA−) | unmutated | wc - synth | 6 | 54 | 5 | 1047 | 25 | 39557 | 8 | 32 | 0 | 1 | 1 |
| D-26 (CA++) | disrupted | wc - synth | 0 | 0 | 0 | 0 | 0 | 5680 | 0 | 0 | 0 | 0 | 0 |

For the M-17 (A−) tract, only 47% of the reads report the expected length of 17 bp. For the M-18 (C−) tract, the results are even worse: 28% of reads report the expected length. In contrast, the M-18 (C++) disrupted templates have 99% of reads on-target. For the D-26 (CA−) unmutated library, 83% of reads report the on-target length. The most frequently reported variants occur at 2 bp increments, equivalent to the size of the repeat unit. In contrast, the disrupted D-26 (CA++) templates have a high on-target rate with 98% reporting a length of 26. Unlike the unmutated D-26 (CA−), the off-target reads in D-26 (CA+) are almost entirely 1 bp off, reporting a length of 25 or 27.

### First Copies

Accuracy can, in principle, be improved by taking a consensus of lengths from reads over first copies. The modal length of a first copy is defined as the value most commonly seen among all the reads associated with it. The distributions of modal lengths are displayed in FIG. 8, in panels F-J, and reported as counts in Table 3, part B. For the M-17 (A−) library, there is a slight improvement in the proportion of MS lengths that are on-target, from 47% when counting reads to 68% when counting the first copy consensus. For the M-18 (C−) library, there is a decline in lengths that are on-target from 28% to 24%. MS length estimates from the D-26 (CA−) unmutated library improve significantly when based on first copies, with 96% on-target, compared to 83% for reads alone. Unexpectedly, the lengths based on disrupted MS have nearly identical on-target rates, about 98% for D-26 (CA++) and 99% for M-18 (C++), whether using reads or first copy consensus. It is suspected that synthetic variant templates are the cause.

The proportion of synthetic variants, off-target length templates created during the synthesis of the original material, would not exceed the proportion of off-target first copies observed in the best data sets. The proportions in the disrupted data are: 2% for the D-26 (CA++) and 1% for the M-18 (C++). For the unmutated mononucleotide M-17 (A−) and M-18 (C−), the off-target rates are so high that they dwarf any potential improvement from removing these synthetic variant templates. However, identifying and removing the synthetic variant templates could dramatically improve the estimations of off-target rates of M-18 (C++), D-26 (CA++), and D-26 (CA−). To resolve this issue, data aggregated over the initial templates for these three libraries is analyzed.

### Templates

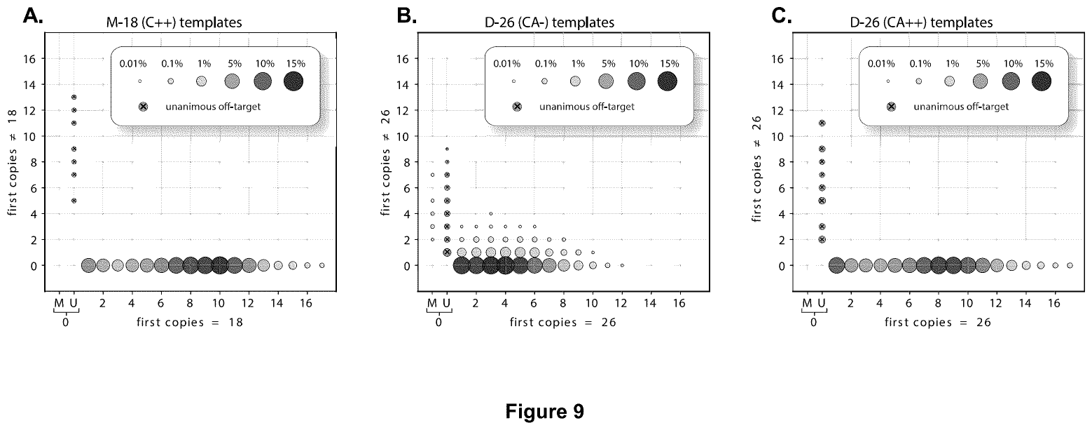

For each initial template, the modal lengths over all of its first copies are tabulated. This information is condensed by counting the number of first copies on-target (x) and the number of first copies that are off-target (y). FIG. 9 shows a scatter plot summarizing the distribution of (x, y) over all templates for each of the three libraries: M-18 (C++), D-26 (CA−), and D-26 (CA++). The size of the dot and darkness of shading reflect the proportion of templates with those values. For templates with no on-target first copies, the population is split between those whose first copies are unanimous for an off-target length (circles with x's through them, column U) and those whose first copies show multiple off-target lengths (column M).

Table 4, below, presents the data underlying FIG. 9, for the libraries M-18 (C++), D-26 (CA−), and D-26 (CA++). Each template is tabulated by the number of well-covered first copies on-target (equal to 18 or 26) and the number of well-covered first copies off-target. For the disrupted libraries, templates have either all first copies on-target or all first copies off-target. For the D-26 (CA−), many templates are mixed with both on-target and off-target first copies.

TABLE 4
On-target and off-target rates of first copies, per template. Companion to FIG. 9.
part 1

| D-26 (CA−) | | well-covered first copies ≠ 26 | | | | | | | | | | | | | | | | | |
|---|---|---|---|---|---|---|---|---|---|---|---|---|---|---|---|---|---|---|---|
| unmutated | | 0 | 1 | 2 | 3 | 4 | 5 | 6 | 7 | 8 | 9 | 10 | 11 | 12 | 13 | 14 | 15 | 16 | 17 |
| well-covered | 0 | 0 | 68 | 22 | 21 | 24 | 20 | 14 | 12 | 3 | 1 | 0 | 0 | 0 | 0 | 0 | 0 | 0 | 0 |
| first | 1 | 1569 | 108 | 6 | 1 | 0 | 0 | 0 | 0 | 0 | 0 | 0 | 0 | 0 | 0 | 0 | 0 | 0 | 0 |
| copies = 26 | 2 | 1454 | 133 | 17 | 1 | 0 | 0 | 0 | 0 | 0 | 0 | 0 | 0 | 0 | 0 | 0 | 0 | 0 | 0 |
| | 3 | 1703 | 163 | 17 | 2 | 1 | 0 | 0 | 0 | 0 | 0 | 0 | 0 | 0 | 0 | 0 | 0 | 0 | 0 |
| | 4 | 1772 | 208 | 11 | 1 | 0 | 0 | 0 | 0 | 0 | 0 | 0 | 0 | 0 | 0 | 0 | 0 | 0 | 0 |
| | 5 | 1519 | 187 | 19 | 1 | 0 | 0 | 0 | 0 | 0 | 0 | 0 | 0 | 0 | 0 | 0 | 0 | 0 | 0 |
| | 6 | 1056 | 143 | 12 | 1 | 0 | 0 | 0 | 0 | 0 | 0 | 0 | 0 | 0 | 0 | 0 | 0 | 0 | 0 |
| | 7 | 674 | 55 | 4 | 0 | 0 | 0 | 0 | 0 | 0 | 0 | 0 | 0 | 0 | 0 | 0 | 0 | 0 | 0 |
| | 8 | 319 | 23 | 3 | 0 | 0 | 0 | 0 | 0 | 0 | 0 | 0 | 0 | 0 | 0 | 0 | 0 | 0 | 0 |
| | 9 | 127 | 15 | 0 | 0 | 0 | 0 | 0 | 0 | 0 | 0 | 0 | 0 | 0 | 0 | 0 | 0 | 0 | 0 |
| | 10 | 43 | 1 | 0 | 0 | 0 | 0 | 0 | 0 | 0 | 0 | 0 | 0 | 0 | 0 | 0 | 0 | 0 | 0 |
| | 11 | 9 | 0 | 0 | 0 | 0 | 0 | 0 | 0 | 0 | 0 | 0 | 0 | 0 | 0 | 0 | 0 | 0 | 0 |
| | 12 | 1 | 0 | 0 | 10 | 0 | 0 | 0 | 0 | 0 | 0 | 0 | 0 | 0 | 0 | 0 | 0 | 0 | 0 |
| | 13 | 0 | 0 | 0 | 0 | 0 | 0 | 0 | 0 | 0 | 0 | 0 | 0 | 0 | 0 | 0 | 0 | 0 | 0 |
| | 14 | 0 | 0 | 0 | 0 | 0 | 0 | 0 | 0 | 0 | 0 | 0 | 0 | 0 | 0 | 0 | 0 | 0 | 0 |
| | 15 | 0 | 0 | 0 | 0 | 0 | 0 | 0 | 0 | 0 | 0 | 0 | 0 | 0 | 0 | 0 | 0 | 0 | 0 |
| | 16 | 0 | 0 | 0 | 0 | 0 | 0 | 0 | 0 | 0 | 0 | 0 | 0 | 0 | 0 | 0 | 0 | 0 | 0 |
| | 17 | 0 | 0 | 0 | 0 | 0 | 0 | 0 | 0 | 0 | 0 | 0 | 0 | 0 | 0 | 0 | 0 | 0 | 0 |

| part 2 | |
|---|---|
| D-26 (CA++) | well-covered first copies ≠ 26 |

| disrupted | | 0 | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|---|---|
| well-covered | 0 | 0 | 0 | 3 | 2 | 0 | 3 | 2 | 1 | 1 | 2 | 0 | 2 | 0 | 0 | 0 | 0 | 0 | 0 |
| first | 1 | 86 | 0 | 0 | 0 | 0 | 0 | 0 | 0 | 0 | 0 | 0 | 0 | 0 | 0 | 0 | 0 | 0 | 0 |
| copies = 26 | 2 | 43 | 0 | 0 | 0 | 0 | 0 | 0 | 0 | 0 | 0 | 0 | 0 | 0 | 0 | 0 | 0 | 0 | 0 |
| | 3 | 41 | 0 | 0 | 0 | 0 | 0 | 0 | 0 | 0 | 0 | 0 | 0 | 0 | 0 | 0 | 0 | 0 | 0 |
| | 4 | 40 | 0 | 0 | 0 | 0 | 0 | 0 | 0 | 0 | 0 | 0 | 0 | 0 | 0 | 0 | 0 | 0 | 0 |
| | 5 | 51 | 0 | 0 | 0 | 0 | 0 | 0 | 0 | 0 | 0 | 0 | 0 | 0 | 0 | 0 | 0 | 0 | 0 |
| | 6 | 61 | 0 | 0 | 0 | 0 | 0 | 0 | 0 | 0 | 0 | 0 | 0 | 0 | 0 | 0 | 0 | 0 | 0 |
| | 7 | 88 | 0 | 0 | 0 | 0 | 0 | 0 | 0 | 0 | 0 | 0 | 0 | 0 | 0 | 0 | 0 | 0 | 0 |
| | 8 | 109 | 0 | 0 | 0 | 0 | 0 | 0 | 0 | 0 | 0 | 0 | 0 | 0 | 0 | 0 | 0 | 0 | 0 |
| | 9 | 104 | 0 | 0 | 0 | 0 | 0 | 0 | 0 | 0 | 0 | 0 | 0 | 0 | 0 | 0 | 0 | 0 | 0 |
| | 10 | 88 | 0 | 0 | 0 | 0 | 0 | 0 | 0 | 0 | 0 | 0 | 0 | 0 | 0 | 0 | 0 | 0 | 0 |
| | 11 | 62 | 0 | 0 | 0 | 0 | 0 | 0 | 0 | 0 | 0 | 0 | 0 | 0 | 0 | 0 | 0 | 0 | 0 |
| | 12 | 35 | 0 | 0 | 0 | 0 | 0 | 0 | 0 | 0 | 0 | 0 | 0 | 0 | 0 | 0 | 0 | 0 | 0 |
| | 13 | 1 | 6 | 0 | 0 | 0 | 0 | 0 | 0 | 0 | 0 | 0 | 0 | 0 | 0 | 0 | 0 | 0 | 0 |
| | 14 | 6 | 0 | 0 | 0 | 0 | 0 | 0 | 0 | 0 | 0 | 0 | 0 | 0 | 0 | 0 | 0 | 0 | 0 |
| | 15 | 3 | 0 | 0 | 0 | 0 | 0 | 0 | 0 | 0 | 0 | 0 | 0 | 0 | 0 | 0 | 0 | 0 | 0 |
| | 16 | 1 | 0 | 0 | 0 | 0 | 0 | 0 | 0 | 0 | 0 | 0 | 0 | 0 | 0 | 0 | 0 | 0 | 0 |
| | 17 | 1 | 0 | 0 | 0 | 0 | 0 | 0 | 0 | 0 | 0 | 0 | 0 | 0 | 0 | 0 | 0 | 0 | 0 |

| part 3 | |
|---|---|
| M-18 (C++) | well-covered first copies ≠ 18 |

| disrupted | | 0 | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|---|---|
| well-covered | 0 | 0 | 0 | 0 | 0 | 0 | 1 | 0 | 1 | 1 | 1 | 0 | 1 | 1 | 1 | 0 | 0 | 0 | 0 |
| first | 1 | 99 | 0 | 0 | 0 | 0 | 0 | 0 | 0 | 0 | 0 | 0 | 0 | 0 | 0 | 0 | 0 | 0 | 0 |
| copies = 26 | 2 | 65 | 0 | 0 | 0 | 0 | 0 | 0 | 0 | 0 | 0 | 0 | 0 | 0 | 0 | 0 | 0 | 0 | 0 |
| | 3 | 38 | 0 | 0 | 0 | 0 | 0 | 0 | 0 | 0 | 0 | 0 | 0 | 0 | 0 | 0 | 0 | 0 | 0 |
| | 4 | 62 | 0 | 0 | 0 | 0 | 0 | 0 | 0 | 0 | 0 | 0 | 0 | 0 | 0 | 0 | 0 | 0 | 0 |
| | 5 | 76 | 0 | 0 | 0 | 0 | 0 | 0 | 0 | 0 | 0 | 0 | 0 | 0 | 0 | 0 | 0 | 0 | 0 |
| | 6 | 104 | 0 | 0 | 0 | 0 | 0 | 0 | 0 | 0 | 0 | 0 | 0 | 0 | 0 | 0 | 0 | 0 | 0 |
| | 7 | 148 | 0 | 0 | 0 | 0 | 0 | 0 | 0 | 0 | 0 | 0 | 0 | 0 | 0 | 0 | 0 | 0 | 0 |
| | 8 | 177 | 0 | 0 | 0 | 0 | 0 | 0 | 0 | 0 | 0 | 0 | 0 | 0 | 0 | 0 | 0 | 0 | 0 |
| | 9 | 188 | 0 | 0 | 0 | 0 | 0 | 0 | 0 | 0 | 0 | 0 | 0 | 0 | 0 | 0 | 0 | 0 | 0 |
| | 10 | 216 | 0 | 0 | 0 | 0 | 0 | 0 | 0 | 0 | 0 | 0 | 0 | 0 | 0 | 0 | 0 | 0 | 0 |
| | 11 | 152 | 0 | 0 | 0 | 0 | 0 | 0 | 0 | 0 | 0 | 0 | 0 | 0 | 0 | 0 | 0 | 0 | 0 |
| | 12 | 98 | 0 | 0 | 0 | 0 | 0 | 0 | 0 | 0 | 0 | 0 | 0 | 0 | 0 | 0 | 0 | 0 | 0 |
| | 13 | 43 | 0 | 0 | 0 | 0 | 0 | 0 | 0 | 0 | 0 | 0 | 0 | 0 | 0 | 0 | 0 | 0 | 0 |
| | 14 | 11 | 0 | 0 | 0 | 0 | 0 | 0 | 0 | 0 | 0 | 0 | 0 | 0 | 0 | 0 | 0 | 0 | 0 |
| | 15 | 9 | 0 | 0 | 0 | 0 | 0 | 0 | 0 | 0 | 0 | 0 | 0 | 0 | 0 | 0 | 0 | 0 | 0 |
| | 16 | 2 | 0 | 0 | 0 | 0 | 0 | 0 | 0 | 0 | 0 | 0 | 0 | 0 | 0 | 0 | 0 | 0 | 0 |
| | 17 | 1 | 0 | 0 | 0 | 0 | 0 | 0 | 0 | 0 | 0 | 0 | 0 | 0 | 0 | 0 | 0 | 0 | 0 |

The templates with disrupted MS, both the mono- and di-nucleotide repeats, fall into two cleanly separable groups: the vast majority, in which the consensus MS length of first copies all unanimously agree with the on-target length; and a much smaller number of outlier templates, in which none of their first copies have an on-target length. All outlier templates were further examined. For each of these, all first copies agree on their unexpected MS length, most commonly one base-pair less than the expected length. The lengths of unanimous templates are shown in Table 5.

TABLE 5
Lengths of unanimous templates
For the three libraries, M-18 (C++), D-26 (CA−), and D-26
(CA++), shown in FIG. 1.7, the number of templates with
unanimous first copies, for each possible length, is tabulated,
separated further by the number of first copies per template.
The vast majority of unanimous templates are of the expected
on-target length (18 or 26). Synthetic variant templates (3+
unanimous first copies) were removed before
the final error rate estimations.

| D-26 (CA−) | | number of first copies | | | |
|---|---|---|---|---|---|
| unmutated | | 1 | 2 | 3 | 4+ |
| unanimous | <=23 | 12 | 2 | 2 | 18 |
| template | 24 | 40 | 4 | 0 | 3 |
| MSL | 25 | 12 | 10 | 12 | 34 |
| | 26 | 1569 | 1454 | 1703 | 5520 |
| | 27 | 2 | 5 | 3 | 12 |
| | 28 | 1 | 0 | 0 | 0 |
| | >=29 | 1 | 0 | 0 | 0 |
| D-26 (CA++) | | number of first copies | | | |
| disrupted | | 1 | 2 | 3 | 4+ |
| unanimous | <=23 | 0 | 1 | 0 | 2 |
| template | 24 | 0 | 0 | 0 | 0 |
| MSL | 25 | 0 | 2 | 2 | 7 |
| | 26 | 86 | 43 | 41 | 665 |
| | 27 | 0 | 0 | 0 | 2 |
| | 28 | 0 | 0 | 0 | 0 |
| | >=29 | 0 | 0 | 0 | 0 |
| M-13 (C++) | | number of first copies | | | |
| disrupted | | 1 | 2 | 3 | 4+ |
| unanimous | <=15 | 0 | 0 | 0 | 2 |
| template | 16 | 0 | 0 | 0 | 1 |
| MSL | 17 | 5 | 0 | 0 | 17 |
| | 18 | 345 | 218 | 167 | 4384 |
| | 19 | 0 | 0 | 1 | 3 |
| | 20 | 0 | 0 | 0 | 0 |
| | >=21 | 0 | 0 | 0 | 0 |

The analysis of the unmutated D-26 (CA−) tract shows a more complex pattern (Table 6, part B). As before, a majority of templates are on-target, with all first copies in agreement. There is also a small proportion of outlier templates, unanimous in that no first copies have the on-target length, and as before, most of these are unanimous for another length, typically one base less than the expected length (see Table 5). It is noteworthy that relatively few of these outlier templates are +/−2 bp the target length, as would be expected from polymerase error. In addition, there is a third and fairly numerous group of templates in D-26 (CA−): those with a predominance of first copies of the on-target length, yet containing one or more first copies with an off-target length.

TABLE 6
parts A-C: Observed and simulated error moments
For the M-18 (C++), D-26 (CA−), and D-26 (CA++)
conditions, the table shows the Luria-Delbruck Diffusion (LDD)
per-round error rate parameter selected to best fit the observed data.
(Left) The results of the simulation are shown as number of reads
and first copies at length 26 and 24 (or 18 and 17), for each condition. (Right) Parallel
results after downsampling from observed data. The simulation total counts are
matched to the downsampled total counts. Further, the table
shows the proportion of first copies at each length, given N number of
unanimous reads for both simulated and downsampled data. Off-target counts
in the disrupted libraries are 0, due to the extremely low error rates of these conditions,
necessitating the estimation of error via simulation to accurately estimate error rates.
part A.

| | | D-26 (CA−) | | | |
|---|---|---|---|---|---|
| | | | | LDD simulated, | |
| | | observed | | α = 1.35E−02 | |
| MS length (L) | | 24 | 26 | 24 | 26 |
| number of reads | | 303,560.00 | 1,910,332.00 | 308,265.08 | 1,920,815.70 |
| number of first copies | | 1,047.00 | 39,557.00 | 957.93 | 39,733.44 |
| N-th | N = 1 | 0.133978 | 0.841325 | 0.135544 | 0.846076 |
| moment | N = 2 | 0.033846 | 0.729053 | 0.029580 | 0.731265 |
| for length | N = 3 | 0.017401 | 0.636207 | 0.013170 | 0.635169 |
| L | N = 4 | 0.013061 | 0.558259 | 0.009098 | 0.553591 |

part B.

| | | D-26 (CA++) | | | |
|---|---|---|---|---|---|
| | | | | LDD simulated, | |
| | | observed | | α = 4.95E−05 | |
| MS length (L) | | 24 | 26 | 24 | 26 |
| number of reads | | 2,453.00 | 4,312,537.00 | 2,482.78 | 4,315,256.48 |
| number of first copies | | 0.00 | 5,680.00 | 0.41 | 5,679.54 |
| N-th | N = 1 | 0.000578 | 0.998725 | 0.000575 | 0.999370 |
| moment | N = 2 | 0.000005 | 0.997459 | 0.000072 | 0.998820 |
| for length | N = 3 | 0.000000 | 0.996201 | 0.000055 | 0.998289 |
| L | N = 4 | 0.000000 | 0.994952 | 0.000050 | 0.997771 |

part C.

| | | M-18 (C++) | | | |
|---|---|---|---|---|---|
| | | | | LDD simulated, | |
| | | observed | | α = 2.80E−05 | |
| MS length (L) | | 17 | 18 | 17 | 18 |
| number of reads | | 509.00 | 1,612,051.00 | 532.80 | 1,612,048.90 |
| number of first copies | | 0.00 | 11,415.00 | 0.52 | 11,414.43 |
| N-th | N = 1 | 0.000324 | 0.999631 | 0.000330 | 0.999637 |
| moment | N = 2 | 0.000005 | 0.999268 | 0.000045 | 0.999323 |
| for length | N = 3 | 0.000000 | 0.998913 | 0.000035 | 0.999020 |
| L | N = 4 | 0.000000 | 0.998563 | 0.000032 | 0.998724 |

### Removing Synthetic Variants

Based on these studies, templates were declared to be synthetic variants if they had three or more first copies which were unanimous for an off-target length. For the three conditions just discussed, MS length error rates were determined following removal of the synthetic variant templates. To do this fairly, after excluding synthetic variant templates, well-covered first copies and the reads associated with them were considered if and only if they derive from well-covered templates, that is, templates with at least three well-covered first copies. MS length read error was therefore estimated from three of the five conditions shown in Table 3, part A, partitioning the error by its deviation from the expected length. The read error rates for disrupted reads in the mutated M-18 (C++) and D-26 (CA++) data are on the order of 10⁻³ or better, but remain at about 16% for the unmutated D-26 (CA−) data. Almost all of the erroneous reads in the D-26 (CA−) have MS lengths of 24. The erroneous reads for D-26 (CA++) are almost equally divided between lengths 24 and 25 (−2 and −1). The former value probably arises from residual tandem repeats following disruption. Consistent with this, error rates as a function of disruption parameters (Table 7) were examined, and it was noted that if these parameters had been more restrictive, the read error rates could be reduced further.

In Table 6, for the two mutated libraries, the read off-target rate, and number of reads retained, were analyzed as a function of the two disruption parameters. The max repeat length is varied from 3 to 18 for the M-18 library and 3 to 13 for the D-26 library, with the conversion rate thresholds varying from 0.45 to 0.55 as the strictest threshold, to 0.05 to 0.95 to the loosest threshold. Highlighted in bold are the values for the parameter range in the main analysis. Error rates can be reduced further by using stricter disruption thresholds, though yield is also affected by this choice.

TABLE 7
Error rates as functions of disruption parameters

| off-target error rate | | | | | | | total reads | | | | | | |
|---|---|---|---|---|---|---|---|---|---|---|---|---|---|
| | | conversion rate | | | | | | | conversion rate | | | | |
| | | 0.45 to | 0.35 to | 0.25 to | 0.15 to | 0.05 to | | | 0.45 to | 0.35 to | 0.25 to | 0.15 to | 0.05 to |
| M-18 (C+) | | 0.55 | 0.65 | 0.75 | 0.85 | 0.95 | M-18 (C+) | | 0.55 | 0.65 | 0.75 | 0.85 | 0.95 |
| max | 3 | 0.00006 | 0.00023 | 0.00019 | 0.00019 | 0.00019 | max | 3 | 16,498 | 142,292 | 209,475 | 209,475 | 209,475 |
| repeat | 4 | 0.00019 | 0.00028 | 0.00030 | 0.00029 | 0.00029 | repeat | 4 | 42,073 | 327,327 | 710,829 | 763,980 | 763,980 |
| length | 5 | 0.00033 | 0.00041 | 0.00039 | 0.00036 | 0.00036 | length | 5 | 53,810 | 478,676 | 1,293,599 | 1,612,051 | 1,612,051 |
| | 6 | 0.00037 | 0.00052 | 0.00052 | 0.00049 | 0.00049 | | 6 | 56,776 | 549,659 | 1,657,838 | 2,314,420 | 2,333,714 |
| | 7 | 0.00046 | 0.00064 | 0.00076 | 0.00076 | 0.00076 | | 7 | 58,761 | 578,090 | 1,912,390 | 3,010,653 | 3,099,788 |
| | 8 | 0.00046 | 0.00081 | 0.00125 | 0.00149 | 0.00152 | | 8 | 58,761 | 586,201 | 2,051,114 | 3,480,046 | 3,677,449 |
| | 9 | 0.00046 | 0.00129 | 0.00244 | 0.00346 | 0.00381 | | 9 | 58,761 | 596,911 | 2,133,730 | 3,845,816 | 4,212,495 |
| | 10 | 0.00046 | 0.00165 | 0.00364 | 0.00657 | 0.00752 | | 10 | 58,761 | 596,937 | 2,161,177 | 4,062,573 | 4,567,219 |
| | 11 | 0.00046 | 0.00165 | 0.00503 | 0.01006 | 0.01256 | | 11 | 58,761 | 596,937 | 2,181,856 | 4,201,911 | 4,829,834 |
| | 12 | 0.00046 | 0.00165 | 0.00603 | 0.01316 | 0.01732 | | 12 | 58,761 | 596,937 | 2,192,165 | 4,278,100 | 4,996,345 |
| | 13 | 0.00046 | 0.00165 | 0.00618 | 0.01533 | 0.02243 | | 13 | 58,761 | 596,937 | 2,193,567 | 4,314,939 | 5,116,197 |
| | 14 | 0.00046 | 0.00165 | 0.00618 | 0.01686 | 0.02748 | | 14 | 58,761 | 596,937 | 2,193,567 | 4,336,144 | 5,199,032 |
| | 15 | 0.00046 | 0.00165 | 0.00618 | 0.01756 | 0.03247 | | 15 | 58,761 | 596,937 | 2,193,567 | 4,343,171 | 5,253,745 |
| | 16 | 0.00046 | 0.00165 | 0.00618 | 0.01756 | 0.03645 | | 16 | 58,761 | 596,937 | 2,193,567 | 4,343,171 | 5,284,561 |
| | 17 | 0.00046 | 0.00165 | 0.00618 | 0.01756 | 0.03913 | | 17 | 58,761 | 596,937 | 2,193,567 | 4,343,171 | 5,303,142 |
| | 18 | 0.00046 | 0.00165 | 0.00618 | 0.01756 | 0.03923 | | 18 | 58,761 | 596,937 | 2,193,567 | 4,343,171 | 5,303,367 |

| off-target error rate | | | | | | | total reads | | | | | | |
|---|---|---|---|---|---|---|---|---|---|---|---|---|---|
| | | conversion rate | | | | | | | conversion rate | | | | |
| | | 0.45 to | 0.35 to | 0.25 to | 0.15 to | 0.05 to | | | 0.45 to | 0.35 to | 0.25 to | 0.15 to | 0.05 to |
| D-26 (CA+) | | 0.55 | 0.65 | 0.75 | 0.85 | 0.95 | D-26 (CA+) | | 0.55 | 0.65 | 0.75 | 0.85 | 0.95 |
| max | 3 | 0.00070 | 0.00072 | 0.00074 | 0.00073 | 0.00073 | max | 3 | 865,694 | 1,418,594 | 1,781,407 | 1,831,219 | 1,831,219 |
| repeat | 4 | 0.00071 | 0.00072 | 0.00076 | 0.00077 | 0.00077 | repeat | 4 | 1,178,692 | 2,105,597 | 2,890,865 | 3,187,149 | 3,187,149 |
| length | 5 | 0.00089 | 0.00126 | 0.00193 | 0.00195 | 0.00195 | length | 5 | 1,299,708 | 2,443,743 | 3,556,909 | 4,313,533 | 4,313,533 |
| | 6 | 0.00154 | 0.00197 | 0.00350 | 0.00469 | 0.00474 | | 6 | 1,355,061 | 2,554,528 | 3,865,372 | 5,021,134 | 5,030,078 |
| | 7 | 0.00190 | 0.00260 | 0.00589 | 0.00933 | 0.00962 | | 7 | 1,361,644 | 2,569,339 | 3,978,289 | 5,397,530 | 5,448,898 |
| | 8 | 0.00190 | 0.00322 | 0.00738 | 0.01381 | 0.01421 | | 8 | 1,361,644 | 2,581,850 | 4,022,127 | 5,575,130 | 5,637,667 |
| | 9 | 0.00190 | 0.00322 | 0.00738 | 0.01722 | 0.01870 | | 9 | 1,361,644 | 2,581,850 | 4,022,127 | 5,620,895 | 5,696,458 |
| | 10 | 0.00190 | 0.00322 | 0.00738 | 0.02062 | 0.02353 | | 10 | 1,361,644 | 2,581,850 | 4,022,127 | 5,662,875 | 5,757,976 |
| | 11 | 0.00190 | 0.00322 | 0.00738 | 0.02173 | 0.02553 | | 11 | 1,361,644 | 2,581,850 | 4,022,127 | 5,674,069 | 5,774,619 |
| | 12 | 0.00190 | 0.00322 | 0.00738 | 0.02173 | 0.02739 | | 12 | 1,361,644 | 2,581,850 | 4,022,127 | 5,674,069 | 5,790,936 |
| | 13 | 0.00190 | 0.00322 | 0.00738 | 0.02173 | 0.02739 | | 13 | 1,361,644 | 2,581,850 | 4,022,127 | 5,674,069 | 5,790,936 |

In Table 3, part B, first copy error is similarly shown, before and after removal for synthetic variants. After removal, there are 11,415 and 5,680 first copies in the M-18 (C++) and D-26 (CA++) data, respectively, 100% of which are of the expected length. First copy error for the CA− is reduced somewhat, but stands at about 3%. Most of the errors are to lengths of 24, two less than the target length, the error expected from slippage of one repeat unit.

### Example 1G: Determining Limits of Detection

As established above, disruption reduces read error rates to 10⁻³ or better. This section demonstrates that multiple disrupted reads, either from the same first copy or preferably from multiple first copies from the same initial template, achieve error rates of 10⁻⁶ or better, whereas without disruption error rates are not better than 10⁻² for CA−, the relatively stable repeat tract.

To measure error rates as a function of multiple reads the method of moments is used. For a given condition and template, the N^{th} moment of L is defined as the probability of observing unanimous agreement of length L for N reads from the same first copy. With moments, the probability of reads unanimous for length L with a configuration of N[j] reads over J first copies from the same template can be estimated by multiplying the N[j] moments of L.

For the three conditions, CA−, CA++, and C++, the moments are shown in Table 6 for both the expected lengths (26, 26 and 18) and for the most commonly observed length error, a 1-unit deletion (24, 24 and 17, respectively). Note that the N-th moments are not powers of the first moment, because the moments account for variation in error rates across first copies. This variation is inherent in exponential growth, a mathematical insight first noted by Luria-Delbruck (Luria, S. E. & Delbrück, 1943).

In the CA-data, the first moment for 24 matches the average read error rate of 1.3×10⁻¹. The second moment for that length in CA-diminishes to 3.4×10⁻² reflecting increased accuracy from two unanimous reads. The third and fourth moments continue the downward trend but will plateau, reflecting that for large N, the moments cannot be lower than the first-round error rate. This is estimated from the data to be about 1.3×10⁻² for CA−.

For the disrupted datasets, CA++ and C++, the first moments for off-target also match the average read error rates of 5.8×10⁻⁴ and 3.2×10⁻⁴, respectively. The probability of seeing two unanimous off-target reads, the 2nd moment, dramatically decreases to 5×10⁻⁶. The higher moments are vanishingly small. However, due to the number of observable first copies (5680 for CA++ and 11415 for C++, see Table 1.3) and low error rate, no first-round error in any copy are observed (FIG. 9), so these values cannot plateau. It was therefore likely that the tail of the distribution that define the higher moments is underestimated by this approach.

To obtain a better approximation for the higher moments, a Luria Delbruck Diffusion (LDD) model that simulates error during amplification is used (see Example 1D for details). In addition to providing a simulation of the higher moments, a good LDD fit to the data can provide an estimate of per round error rate. With a good estimate of the per round error rate, one can also accurately simulate any number of rounds of amplification.

A simple LDD model has four parameters: efficiency of replication (e=0.95), the number of rounds of replication (R=23), and two per round length error rates: one unit decrease (α), and one unit increase (β). For each dataset, LDD distributions are numerically generated over a grid of α and β, and the grid-point where the read error-rates (1^{st} moments) best match the empirical data are selected. Table 6 also shows the best-fit a parameter, the per round error of decreasing by one repeat unit, for each dataset along with simulations for the read and first copy error rates and the four moments.

For the CA− data, a per-round error rate of 1.36×10⁻² was estimated. As this was chosen to match the read-error rate, a good match for the first moment is seen. However, the corresponding LDD distribution also recapitulates the first copy rates and the higher moments of the sample distribution. For the disrupted datasets, CA++ and C++, the per-round error rate of 4.95×10⁻⁵ and 2.80×10⁻⁵, respectively was estimated. The expected number of first copy errors for CA++ and C++ given the size of these datasets are 0.41 (out of 5,680) and 0.52 (out of 11,415), respectively, whereas none were in fact observed. The LDD simulated estimates for the higher moments are therefore higher than the estimates made from the observed distributions.

Using the LDD sample moments, it is possible to estimate detection limits for various unanimous read configurations. Six examples are shown in Table 8 for CA− and CA++ templates. It is clear that the disrupted microsatellites are measured with at least three orders of magnitude lower error than undisrupted microsatellites. Moreover, obtaining reads from the same first copy has a higher error rate than the same number of reads from multiple first copies. Given three unanimous reads over two first copies, less than one in ten million CA++ templates with MSL of 26 would be mistaken as having a MSL of 24. Under conditions of disruption, a limit of detection is estimated to be at or below one in a million, enabling highly sensitive detection of rare microsatellite length variants in biological samples.

TABLE 8
Error rates with unanimous lengths by condition and read configuration
For the D-26 (CA−) and D-26 (CA++) conditions, the LDD simulation
results are used to show expected rates of observing length 24 in
error, depending on the number of first copies and the read configuration
across those first copies. Error rates are orders of magnitude lower
when reads are distributed over multiple first copies.

| | | | D-26 (CA−) | | D-26 (CA++) | |
|---|---|---|---|---|---|---|
| | | first | unmutated | | disrupted | |
| configuration | reads | copies | 24 | 26 | 24 | 26 |
| 1 | 1 | 1 | 1.4E−01 | 0.846 | 5.7E−04 | 0.999 |
| 2 | 2 | 1 | 3.0E−02 | 0.731 | 7.2E−05 | 0.999 |
| 3 | 3 | 1 | 1.3E−02 | 0.635 | 5.5E−05 | 0.998 |
| 1, 1 | 2 | 2 | 1.8E−02 | 0.716 | 3.3E−07 | 0.999 |
| 2, 1 | 3 | 2 | 4.0E−03 | 0.619 | 4.2E−08 | 0.998 |
| 1, 1, 1 | 3 | 3 | 2.5E−03 | 0.606 | 1.9E−10 | 0.998 |

### Example 2—Preparation and Analysis Steps of a Disrupted Microsatellite Library

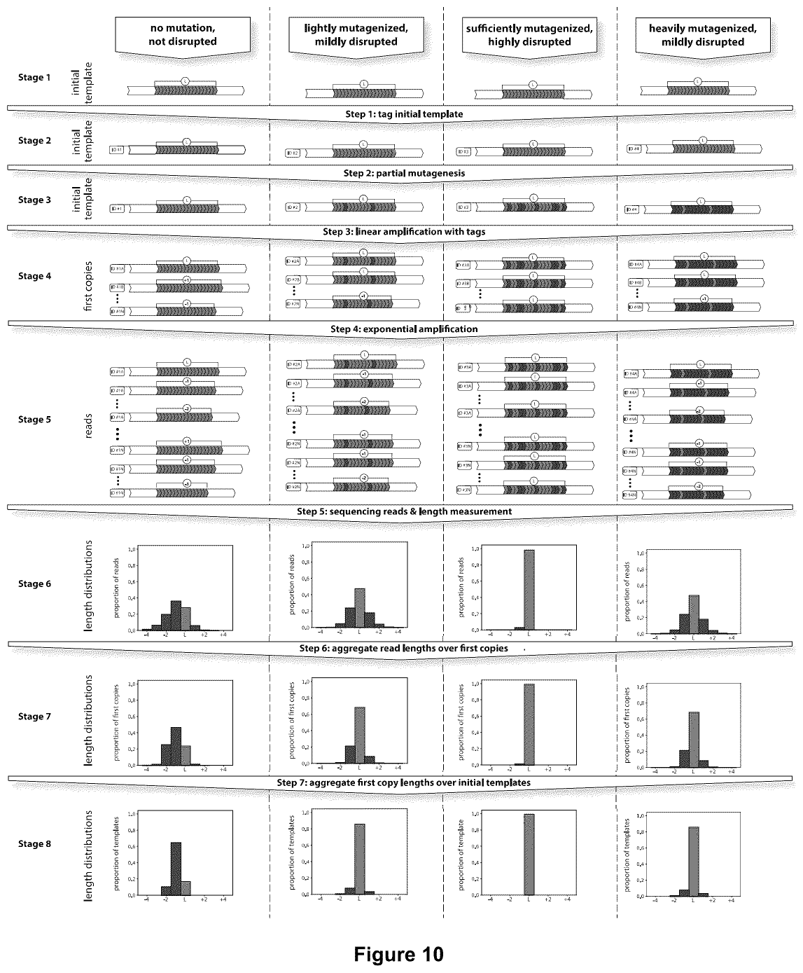

This example and corresponding FIG. 10 demonstrate the library preparation and analysis steps of a library of templates with disrupted microsatellites, beginning from biological specimen DNA. It illustrates the impact of mild and heavy disruption on the stability of microsatellite lengths during this process.

### Structure of the Initial Templates.

Initial templates can be prepared as single or double stranded DNA fragments. In this example, they are extracted from biological specimens. They can be enriched for fragments corresponding to certain genomic loci. The templates of interest have the following structural features: a microsatellite (i.e., a direct repeat motif structure, with N motifs, indicated by the arrows) and flanking sequences on either side of the microsatellite that delineate the ends of the microsatellite region and thereby determining the length L of the repeat region (as indicated in the figure). The purpose of the procedures that follow is to allow the determination of this length L accurately (i.e., with low error).

### Step 1. Tagging the Initial Templates.

Initial templates are tagged in a way such that individual templates can be distinguished, and subsequent copies retain information from the tags. When initial templates are synthetic, they may be synthesized with individual tags from the beginning, in which case a step of tagging is not needed. Without limitation, the individual tags, also referred to herein as “individual identifiers”, may be any combination of: (1) randomized nucleotide sequences added to the ends of templates by extension or ligation (i.e., varietal tags); (2) diverse fragment end positions created during extraction from biological specimens or following treatment with non-specific endonucleases; or (3) a pattern of partial mutation, as introduced into the initial template in the next step. Thus, a step of tagging is not absolutely required by the method if diverse fragment end positions, or a pattern of partial mutation, is used as an individual identifier. As a result of this tagging, two or more copies derived from the same initial template can be so identified. Aggregating read information over identifiable templates reduces error in determining the length of initial templates (see steps 6 through 8)

### Step 2. Partial Mutagenesis of the Initial Templates.

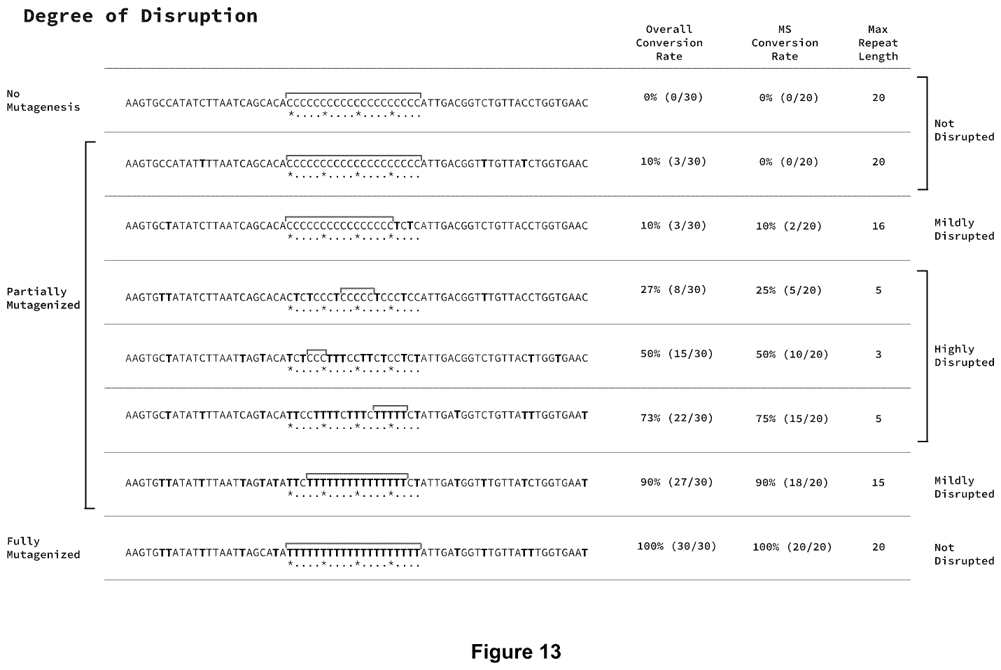

These templates are mutagenized or further mutagenized with the aim of causing disruption of the microsatellite tandem repeat structure. This can be an enzymatic or chemical procedure that alters the base pairing functionality of the initial template. In this example, the process of mutagenesis does not alter the length L between the flanks or the number of repeat units. However, the degree of change to the microsatellite might vary between different templates. Some might have no mutation, and so the structure is not disrupted (left column); some might be lightly mutagenized, resulting in a mildly disrupted repeat structure; some might be sufficiently mutagenized that the structure is highly disrupted; and some might be heavily mutagenized so that a mildly disrupted repeat structure re-emerges (right most column). This happens, for example, if almost all the C's are converted to U's. The degree of microsatellite disruption determines the replicative error of length. (See Example 1, Table 7 and Discussion, FIG. 13)

Step 3. Linear Amplification with New Tags.

This step is optional to the method, but making multiple distinct first copies by linear amplification, and collecting read information from copies of distinct first copies reduces the error in determining the length of initial templates (see steps 6, 7 and 8). This is because error rates compound during exponential amplification. If first copies are made, and each first copy is tagged individually, it is possible to determine when two sequence reads came from the same first copy. The first copies can contain errors in length, as indicated in the diagram, but these errors are reduced in templates with highly disrupted tandem repeat structures.

### Step 4. Exponential Amplification

Exponential amplification generates many more copies of each initial template or of the first copies of the initial templates. These copies retain the identify information (i.e. ID #3A) that was present on the molecules before exponential amplification. This step may involve 5 to 40 rounds of polymerase chain reaction (PCR), generating on the order of 2\hat{ }K copies, where K is the number of rounds of PCR. The high error rate of copying microsatellites leads to a high probability of the length L in a copy being different from its predecessor molecule (indicated as −1, +1, etc. in circles). Highly disrupted molecules are significantly more stable during this process, resulting in few changes in microsatellite lengths, as demonstrated in the diagram. Exponential amplification, or some other amplification process, is generally needed to generate enough molecules to make sequencing libraries and satisfy the input conditions of the sequencing platform.

### Step 5. Sequencing Reads and Length Measurement

From the many copies of the template present at step 5, some number are read out by the sequencing platform as sequencing reads. The length of the microsatellite in a sequencing read is determined by the difference in the position of the left and right flank in the read (FIG. 2). Both the left and right flank sequence (flanking portions) should be at least partially identified in a read to determine its microsatellite length L. The step 6 histograms show the distribution of read lengths observed for a template of length L, under the different disruption paradigms. The proportion of reads with MS lengths that match the length of the initial template (lighter grey) increases with mild disruption, and drastically increases with high levels of disruption.

### Step 6. Aggregate Read Lengths Over First Copies

Reads are aggregated by their first copy identity and a consensus length is determined for each first copy. These first copy consensus lengths are plotted in the step 7 histograms. Error in length measurement is reduced when looking at first copy consensus lengths compared to read lengths alone.

### Step 7. Aggregate First Copy Lengths Over Initial Templates

A further aggregation can then be done over all first copies with the same template identity to determine the template consensus length, shown in step 8. There are many ways to determine a consensus length, including but not limited to taking the plurality length, or requiring a majority length. See Example 1C for a more detailed description of possible sequence processing.

### Example 3—Enrichment of Microsatellite Loci in a Sequencing Library

This example discusses the process of enrichment of microsatellite loci in a sequencing library. It describes a possible algorithm for selecting loci for the enrichment panel; a protocol for generating enriched sequencing libraries; the analysis steps for processing sequencing reads; the method performance; and possible variations on the method.

### Example 3a: Selection of Microsatellite Loci for Enrichment Panel

The composition of panels for enrichment of two classes of microsatellites, mononucleotide C tracts, and dinucleotide AC tracts, are illustrated.

The properties for a collection of oligonucleotides used to comprise a panel and enrich microsatellites are: each oligonucleotide should flank a microsatellite locus; be of length 30 or greater to ensure good hybridization capture; the microsatellites enriched should not be so short that its length is stable under replication; the flank to flank distance should not be so long that the distance between them does not fit into a single read of a short read sequencing platform; the flank sequences themselves should map uniquely, either to the whole genome, or to the set of flanks enriched by the panel; and flanks should not contain within them sequences that would promote spurious capture of other parts of the genome and thereby reduce the purity of the captured product.

An initial list of microsatellite (MS) loci in the hg19 human reference genome was downloaded from the MicroSatellite DataBase (Avvaru, A. K., et al., 2020). C and AC microsatellites of total length 12 to 64 were selected for further consideration (N=169,926). Microsatellites on alternate haplotype contigs were excluded (leaving N=168,551). A series of checks on the uniqueness of 60 base pair flanks in the reference genome were employed to reduce the list. The following criteria were applied to both flanks: (1) all length 21 subsequences of the 60 base left and right MS flanks are unique in the hg19 reference genome (N=26,682); (2) the minimum hamming distance of the each of the 60 base flanks to the rest of the reference genome (and its reverse complement) is at least 20 (N=15,405); (3) 60 base flank sequences mapped using Bowtie2 to the hg38 reference genome, allowing indels, map only once and at the same separation as in hg19 (N=15,313). In addition, all microsatellite loci within 200 bases of another were excluded (N=14,910), and loci within or nearby the PAR regions on chromosome X and Y were excluded (N=14,893). These criteria can be relaxed or made more stringent, or additional criteria added.

From this filtered list, the two flanks from 630 C-MS and 630 AC-MS loci selected at random were chosen to make two panels, and then to test the utility of panels. Larger or smaller sets of flanks could be made. The composition of this panel is found in Table 9 (C-MS) and Table 10 (AC-MS).

TABLE 9
List of capture probe sequences in C panel
Genomic coordinates, sequences, and strand information are listed for microsatellite
flanking sequences of 630 loci containing mononucleotide-C microsatellites, used in a
hybridization capture panel.

| | start | end | | | | 5′ flank | 3′ flank | |
|---|---|---|---|---|---|---|---|---|
| | position | position | | length in | | SEQ ID | SEQ ID | MS SEQ |
| chr | (hg38) | (hg38) | strand | reference | locus id | NO | NO | ID NO |
| chr1 | 1028320 | 1028336 | + | 16 | C-1 | 1 | 631 | 1261 |
| chr1 | 1302795 | 1302807 | + | 12 | C-2 | 2 | 632 | 1262 |
| chr1 | 3900328 | 3900346 | + | 18 | C-3 | 3 | 633 | 1263 |
| chr1 | 4329942 | 4329954 | − | 12 | C-4 | 4 | 634 | 1264 |
| chr1 | 6260376 | 6260390 | + | 14 | C-5 | 5 | 635 | 1265 |
| chr1 | 7230432 | 7230444 | + | 12 | C-6 | 6 | 636 | 1266 |
| chr1 | 7493643 | 7493658 | − | 15 | C-7 | 7 | 637 | 1267 |
| chr1 | 8018373 | 8018387 | − | 14 | C-8 | 8 | 638 | 1268 |
| chr1 | 8350635 | 8350649 | + | 14 | C-9 | 9 | 639 | 1269 |
| chr1 | 10537341 | 10537353 | + | 12 | C-10 | 10 | 640 | 1270 |
| chr1 | 18528054 | 18528071 | − | 17 | C-11 | 11 | 641 | 1271 |
| chr1 | 19963046 | 19963059 | + | 13 | C-12 | 12 | 642 | 1272 |
| chr1 | 21634058 | 21634073 | − | 15 | C-13 | 13 | 643 | 1273 |
| chr1 | 22720660 | 22720673 | + | 13 | C-14 | 14 | 644 | 1274 |
| chr1 | 23531350 | 23531367 | + | 17 | C-15 | 15 | 645 | 1275 |
| chr1 | 24187434 | 24187446 | − | 12 | C-16 | 16 | 646 | 1276 |
| chr1 | 24643482 | 24643496 | + | 14 | C-17 | 17 | 647 | 1277 |
| chr1 | 24869979 | 24869992 | + | 13 | C-18 | 18 | 648 | 1278 |
| chr1 | 25090366 | 25090379 | − | 13 | C-19 | 19 | 649 | 1279 |
| chr1 | 26306829 | 26306841 | − | 12 | C-20 | 20 | 650 | 1280 |
| chr1 | 26993577 | 26993595 | − | 18 | C-21 | 21 | 651 | 1281 |
| chr1 | 27602986 | 27602998 | + | 12 | C-22 | 22 | 652 | 1282 |
| chr1 | 30893303 | 30893317 | + | 14 | C-23 | 23 | 653 | 1283 |
| chr1 | 33742577 | 33742590 | + | 13 | C-24 | 24 | 654 | 1284 |
| chr1 | 40668491 | 40668504 | − | 13 | C-25 | 25 | 655 | 1285 |
| chr1 | 44010456 | 44010471 | − | 15 | C-26 | 26 | 656 | 1286 |
| chr1 | 45629454 | 45629467 | + | 13 | C-27 | 27 | 657 | 1287 |
| chr1 | 51236498 | 51236511 | + | 13 | C-28 | 28 | 658 | 1288 |
| chr1 | 51306853 | 51306865 | + | 12 | C-29 | 29 | 659 | 1289 |
| chr1 | 52263753 | 52263768 | + | 15 | C-30 | 30 | 660 | 1290 |
| chr1 | 57061228 | 57061240 | − | 12 | C-31 | 31 | 661 | 1291 |
| chr1 | 68231857 | 68231872 | − | 15 | C-32 | 32 | 662 | 1292 |
| chr1 | 87331063 | 87331079 | + | 16 | C-33 | 33 | 663 | 1293 |
| chr1 | 92441444 | 92441456 | + | 12 | C-34 | 34 | 664 | 1294 |
| chr1 | 93988609 | 93988622 | − | 13 | C-35 | 35 | 665 | 1295 |
| chr1 | 99265144 | 99265156 | + | 12 | C-36 | 36 | 666 | 1296 |
| chr1 | 109483792 | 109483806 | − | 14 | C-37 | 37 | 667 | 1297 |
| chr1 | 155053107 | 155053120 | + | 13 | C-38 | 38 | 668 | 1298 |
| chr1 | 156248531 | 156248549 | + | 18 | C-39 | 39 | 669 | 1299 |
| chr1 | 158014680 | 158014693 | − | 13 | C-40 | 40 | 670 | 1300 |
| chr1 | 163071855 | 163071873 | + | 18 | C-41 | 41 | 671 | 1301 |
| chr1 | 164618297 | 164618310 | − | 13 | C-42 | 42 | 672 | 1302 |
| chr1 | 164674847 | 164674862 | + | 15 | C-43 | 43 | 673 | 1303 |
| chr1 | 166157036 | 166157049 | − | 13 | C-44 | 44 | 674 | 1304 |
| chr1 | 166973612 | 166973625 | + | 13 | C-45 | 45 | 675 | 1305 |
| chr1 | 201428047 | 201428060 | + | 13 | C-46 | 46 | 676 | 1306 |
| chr1 | 204412052 | 204412079 | − | 27 | C-47 | 47 | 677 | 1307 |
| chr1 | 204482109 | 204482126 | − | 17 | C-48 | 48 | 678 | 1308 |
| chr1 | 205457567 | 205457580 | − | 13 | C-49 | 49 | 679 | 1309 |
| chr1 | 212609375 | 212609388 | − | 13 | C-50 | 50 | 680 | 1310 |
| chr1 | 214465035 | 214465050 | + | 15 | C-51 | 51 | 681 | 1311 |
| chr1 | 215568485 | 215568500 | + | 15 | C-52 | 52 | 682 | 1312 |
| chr1 | 217051204 | 217051216 | − | 12 | C-53 | 53 | 683 | 1313 |
| chr1 | 218489116 | 218489128 | + | 12 | C-54 | 54 | 684 | 1314 |
| chr1 | 224205632 | 224205644 | + | 12 | C-55 | 55 | 685 | 1315 |
| chr1 | 225857384 | 225857397 | − | 13 | C-56 | 56 | 686 | 1316 |
| chr1 | 228208907 | 228208919 | − | 12 | C-57 | 57 | 687 | 1317 |
| chr1 | 228848501 | 228848514 | + | 13 | C-58 | 58 | 688 | 1318 |
| chr1 | 234429764 | 234429777 | − | 13 | C-59 | 59 | 689 | 1319 |
| chr1 | 237833351 | 237833363 | + | 12 | C-60 | 60 | 690 | 1320 |
| chr1 | 240967568 | 240967581 | − | 13 | C-61 | 61 | 691 | 1321 |
| chr1 | 242132192 | 242132207 | − | 15 | C-62 | 62 | 692 | 1322 |
| chr1 | 243473922 | 243473939 | − | 17 | C-63 | 63 | 693 | 1323 |
| chr1 | 243918679 | 243918691 | + | 12 | C-64 | 64 | 694 | 1324 |
| chr1 | 247347581 | 247347596 | + | 15 | C-65 | 65 | 695 | 1325 |
| chr2 | 4782008 | 4782020 | − | 12 | C-66 | 66 | 696 | 1326 |
| chr2 | 10659341 | 10659354 | − | 13 | C-67 | 67 | 697 | 1327 |
| chr2 | 16117209 | 16117221 | + | 12 | C-68 | 68 | 698 | 1328 |
| chr2 | 18747547 | 18747560 | − | 13 | C-69 | 69 | 699 | 1329 |
| chr2 | 23379446 | 23379467 | − | 21 | C-70 | 70 | 700 | 1330 |
| chr2 | 23525726 | 23525738 | + | 12 | C-71 | 71 | 701 | 1331 |
| chr2 | 25230790 | 25230807 | − | 17 | C-72 | 72 | 702 | 1332 |
| chr2 | 28394137 | 28394150 | + | 13 | C-73 | 73 | 703 | 1333 |
| chr2 | 46393945 | 46393960 | − | 15 | C-74 | 74 | 704 | 1334 |
| chr2 | 47172807 | 47172823 | − | 16 | C-75 | 75 | 705 | 1335 |
| chr2 | 48344840 | 48344852 | + | 12 | C-76 | 76 | 706 | 1336 |
| chr2 | 48752981 | 48752996 | − | 15 | C-77 | 77 | 707 | 1337 |
| chr2 | 51001234 | 51001246 | − | 12 | C-78 | 78 | 708 | 1338 |
| chr2 | 60410927 | 60410942 | − | 15 | C-79 | 79 | 709 | 1339 |
| chr2 | 61538041 | 61538054 | + | 13 | C-80 | 80 | 710 | 1340 |
| chr2 | 64987648 | 64987662 | + | 14 | C-81 | 81 | 711 | 1341 |
| chr2 | 66889220 | 66889232 | + | 12 | C-82 | 82 | 712 | 1342 |
| chr2 | 69644165 | 69644182 | + | 17 | C-83 | 83 | 713 | 1343 |
| chr2 | 86068386 | 86068398 | − | 12 | C-84 | 84 | 714 | 1344 |
| chr2 | 98770352 | 98770364 | + | 12 | C-85 | 85 | 715 | 1345 |
| chr2 | 103873541 | 103873554 | + | 13 | C-86 | 86 | 716 | 1346 |
| chr2 | 153872619 | 153872634 | − | 15 | C-87 | 87 | 717 | 1347 |
| chr2 | 170033581 | 170033595 | + | 14 | C-88 | 88 | 718 | 1348 |
| chr2 | 170817225 | 170817238 | + | 13 | C-89 | 89 | 719 | 1349 |
| chr2 | 175620814 | 175620826 | − | 12 | C-90 | 90 | 720 | 1350 |
| chr2 | 190860921 | 190860933 | + | 12 | C-91 | 91 | 721 | 1351 |
| chr2 | 207113607 | 207113620 | − | 13 | C-92 | 92 | 722 | 1352 |
| chr2 | 216803881 | 216803893 | − | 12 | C-93 | 93 | 723 | 1353 |
| chr2 | 217393488 | 217393500 | + | 12 | C-94 | 94 | 724 | 1354 |
| chr2 | 222318771 | 222318783 | + | 12 | C-95 | 95 | 725 | 1355 |
| chr2 | 222559161 | 222559175 | + | 14 | C-96 | 96 | 726 | 1356 |
| chr2 | 226030493 | 226030506 | − | 13 | C-97 | 97 | 727 | 1357 |
| chr2 | 230752029 | 230752043 | + | 14 | C-98 | 98 | 728 | 1358 |
| chr2 | 240724934 | 240724951 | − | 17 | C-99 | 99 | 729 | 1359 |
| chr2 | 241095060 | 241095075 | + | 15 | C-100 | 100 | 730 | 1360 |
| chr2 | 241236977 | 241236994 | − | 17 | C-101 | 101 | 731 | 1361 |
| chr3 | 12985490 | 12985502 | + | 12 | C-102 | 102 | 732 | 1362 |
| chr3 | 13145804 | 13145816 | − | 12 | C-103 | 103 | 733 | 1363 |
| chr3 | 17251425 | 17251438 | + | 13 | C-104 | 104 | 734 | 1364 |
| chr3 | 24198452 | 24198466 | + | 14 | C-105 | 105 | 735 | 1365 |
| chr3 | 31516706 | 31516718 | + | 12 | C-106 | 106 | 736 | 1366 |
| chr3 | 37453115 | 37453130 | + | 15 | C-107 | 107 | 737 | 1367 |
| chr3 | 41961550 | 41961570 | + | 20 | C-108 | 108 | 738 | 1368 |
| chr3 | 48154362 | 48154374 | + | 12 | C-109 | 109 | 739 | 1369 |
| chr3 | 49051584 | 49051596 | + | 12 | C-110 | 110 | 740 | 1370 |
| chr3 | 50321815 | 50321828 | − | 13 | C-111 | 111 | 741 | 1371 |
| chr3 | 53816133 | 53816145 | + | 12 | C-112 | 112 | 742 | 1372 |
| chr3 | 59879919 | 59879932 | + | 13 | C-113 | 113 | 743 | 1373 |
| chr3 | 62215546 | 62215558 | + | 12 | C-114 | 114 | 744 | 1374 |
| chr3 | 62402239 | 62402251 | − | 12 | C-115 | 115 | 745 | 1375 |
| chr3 | 66182716 | 66182728 | − | 12 | C-116 | 116 | 746 | 1376 |
| chr3 | 70973835 | 70973848 | + | 13 | C-117 | 117 | 747 | 1377 |
| chr3 | 71009155 | 71009168 | − | 13 | C-118 | 118 | 748 | 1378 |
| chr3 | 71389238 | 71389252 | − | 14 | C-119 | 119 | 749 | 1379 |
| chr3 | 82010525 | 82010537 | − | 12 | C-120 | 120 | 750 | 1380 |
| chr3 | 108074711 | 108074723 | − | 12 | C-121 | 121 | 751 | 1381 |
| chr3 | 120560065 | 120560080 | + | 15 | C-122 | 122 | 752 | 1382 |
| chr3 | 123373758 | 123373771 | + | 13 | C-123 | 123 | 753 | 1383 |
| chr3 | 124045749 | 124045762 | − | 13 | C-124 | 124 | 754 | 1384 |
| chr3 | 126343152 | 126343179 | + | 27 | C-125 | 125 | 755 | 1385 |
| chr3 | 126815643 | 126815656 | + | 13 | C-126 | 126 | 756 | 1386 |
| chr3 | 128491208 | 128491220 | + | 12 | C-127 | 127 | 757 | 1387 |
| chr3 | 129121068 | 129121082 | + | 14 | C-128 | 128 | 758 | 1388 |
| chr3 | 133827746 | 133827761 | + | 15 | C-129 | 129 | 759 | 1389 |
| chr3 | 156720841 | 156720853 | + | 12 | C-130 | 130 | 760 | 1390 |
| chr3 | 157860651 | 157860665 | − | 14 | C-131 | 131 | 761 | 139 |
| chr3 | 158105032 | 158105044 | + | 12 | C-132 | 132 | 762 | 1392 |
| chr3 | 191329942 | 191329956 | − | 14 | C-133 | 133 | 763 | 1393 |
| chr4 | 1570950 | 1570962 | − | 12 | C-134 | 134 | 764 | 1394 |
| chr4 | 3463450 | 3463462 | − | 12 | C-135 | 135 | 765 | 1395 |
| chr4 | 4857065 | 4857082 | + | 17 | C-136 | 136 | 766 | 1396 |
| chr4 | 7536832 | 7536844 | − | 12 | C-137 | 137 | 767 | 1397 |
| chr4 | 8143159 | 8143171 | − | 12 | C-138 | 138 | 768 | 1398 |
| chr4 | 23221317 | 23221331 | + | 14 | C-139 | 139 | 769 | 1399 |
| chr4 | 30717034 | 30717055 | − | 21 | C-140 | 140 | 770 | 1400 |
| chr4 | 37335300 | 37335312 | − | 12 | C-141 | 141 | 771 | 1401 |
| chr4 | 37797883 | 37797896 | − | 13 | C-142 | 142 | 772 | 1402 |
| chr4 | 41216419 | 41216442 | − | 23 | C-143 | 143 | 773 | 1403 |
| chr4 | 48687492 | 48687506 | − | 14 | C-144 | 144 | 774 | 1404 |
| chr4 | 54440079 | 54440094 | − | 15 | C-145 | 145 | 775 | 1405 |
| chr4 | 102799832 | 102799844 | − | 12 | C-146 | 146 | 776 | 1406 |
| chr4 | 110198074 | 110198087 | + | 13 | C-147 | 147 | 777 | 1407 |
| chr4 | 110631414 | 110631427 | − | 13 | C-148 | 148 | 778 | 1408 |
| chr4 | 112324974 | 112324986 | − | 12 | C-149 | 149 | 779 | 1409 |
| chr4 | 112652293 | 112652306 | + | 13 | C-150 | 150 | 780 | 1410 |
| chr4 | 117494525 | 117494537 | + | 12 | C-151 | 151 | 781 | 1411 |
| chr4 | 127578796 | 127578808 | − | 12 | C-152 | 152 | 782 | 1412 |
| chr4 | 140151690 | 140151702 | − | 12 | C-153 | 153 | 783 | 1413 |
| chr4 | 154743125 | 154743138 | + | 13 | C-154 | 154 | 784 | 1414 |
| chr4 | 155747961 | 155747979 | + | 18 | C-155 | 155 | 785 | 1415 |
| chr4 | 157076972 | 157076986 | − | 14 | C-156 | 156 | 786 | 1416 |
| chr4 | 160546263 | 160546275 | + | 12 | C-157 | 157 | 787 | 1417 |
| chr4 | 163858101 | 163858114 | − | 13 | C-158 | 158 | 788 | 1418 |
| chr4 | 173530413 | 173530425 | − | 12 | C-159 | 159 | 789 | 1419 |
| chr4 | 183256884 | 183256903 | + | 19 | C-160 | 160 | 790 | 1420 |
| chr4 | 184785606 | 184785621 | − | 15 | C-161 | 161 | 791 | 1421 |
| chr4 | 186892130 | 186892142 | − | 12 | C-162 | 162 | 792 | 1422 |
| chr5 | 89887 | 89899 | − | 12 | C-163 | 163 | 793 | 1423 |
| chr5 | 1752339 | 1752354 | − | 15 | C-164 | 164 | 794 | 1424 |
| chr5 | 1795802 | 1795815 | + | 13 | C-165 | 165 | 795 | 1425 |
| chr5 | 2609996 | 2610010 | − | 14 | C-166 | 166 | 796 | 1426 |
| chr5 | 14457373 | 14457387 | + | 14 | C-167 | 167 | 797 | 1427 |
| chr5 | 34840237 | 34840250 | − | 13 | C-168 | 168 | 798 | 1428 |
| chr5 | 41005413 | 41005425 | + | 12 | C-169 | 169 | 799 | 1429 |
| chr5 | 93960081 | 93960095 | + | 14 | C-170 | 170 | 800 | 1430 |
| chr5 | 97065048 | 97065061 | − | 13 | C-171 | 171 | 801 | 1431 |
| chr5 | 97182778 | 97182793 | − | 15 | C-172 | 172 | 802 | 1432 |
| chr5 | 120463788 | 120463800 | + | 12 | C-173 | 173 | 803 | 1433 |
| chr5 | 135216465 | 135216477 | + | 12 | C-174 | 174 | 804 | 1434 |
| chr5 | 136134037 | 136134053 | − | 16 | C-175 | 175 | 805 | 1435 |
| chr5 | 137079533 | 137079545 | + | 12 | C-176 | 176 | 806 | 1436 |
| chr5 | 138897294 | 138897308 | + | 14 | C-177 | 177 | 807 | 1437 |
| chr5 | 139637353 | 139637365 | + | 12 | C-178 | 178 | 808 | 1438 |
| chr5 | 140557095 | 140557108 | + | 13 | C-179 | 179 | 809 | 1439 |
| chr5 | 154191435 | 154191448 | + | 13 | C-180 | 180 | 810 | 1440 |
| chr5 | 158940763 | 158940779 | + | 16 | C-181 | 181 | 811 | 1441 |
| chr5 | 160133966 | 160133979 | + | 13 | C-182 | 182 | 812 | 1442 |
| chr5 | 168165648 | 168165664 | + | 16 | C-183 | 183 | 813 | 1443 |
| chr5 | 172189236 | 172189248 | − | 12 | C-184 | 184 | 814 | 1444 |
| chr5 | 174223802 | 174223814 | − | 12 | C-185 | 185 | 815 | 1445 |
| chr5 | 175116420 | 175116434 | − | 14 | C-186 | 186 | 816 | 1446 |
| chr5 | 176642851 | 176642865 | + | 14 | C-187 | 187 | 817 | 1447 |
| chr5 | 177300057 | 177300070 | + | 13 | C-188 | 188 | 818 | 1448 |
| chr5 | 177302735 | 177302747 | + | 12 | C-189 | 189 | 819 | 1449 |
| chr5 | 178246728 | 178246740 | − | 12 | C-190 | 190 | 820 | 1450 |
| chr5 | 178627217 | 178627230 | + | 13 | C-191 | 191 | 821 | 1451 |
| chr5 | 180101351 | 180101365 | − | 14 | C-192 | 192 | 822 | 1452 |
| chr6 | 1610015 | 1610029 | + | 14 | C-193 | 193 | 823 | 1453 |
| chr6 | 4674348 | 4674364 | − | 16 | C-194 | 194 | 824 | 1454 |
| chr6 | 6713579 | 6713592 | + | 13 | C-195 | 195 | 825 | 1455 |
| chr6 | 7106506 | 7106519 | − | 13 | C-196 | 196 | 826 | 1456 |
| chr6 | 10411046 | 10411058 | + | 12 | C-197 | 197 | 827 | 1457 |
| chr6 | 12291217 | 12291229 | + | 12 | C-198 | 198 | 828 | 1458 |
| chr6 | 12845429 | 12845443 | + | 14 | C-199 | 199 | 829 | 1459 |
| chr6 | 15304303 | 15304318 | + | 15 | C-200 | 200 | 830 | 1460 |
| chr6 | 17496027 | 17496039 | − | 12 | C-201 | 201 | 831 | 1461 |
| chr6 | 19794138 | 19794150 | + | 12 | C-202 | 202 | 832 | 1462 |
| chr6 | 23655986 | 23656001 | − | 15 | C-203 | 203 | 833 | 1463 |
| chr6 | 28988893 | 28988905 | − | 12 | C-204 | 204 | 834 | 1464 |
| chr6 | 35280516 | 35280529 | + | 13 | C-205 | 205 | 835 | 1465 |
| chr6 | 44072606 | 44072618 | + | 12 | C-206 | 206 | 836 | 1466 |
| chr6 | 47529183 | 47529200 | + | 17 | C-207 | 207 | 837 | 1467 |
| chr6 | 51804359 | 51804373 | − | 14 | C-208 | 208 | 838 | 1468 |
| chr6 | 53925134 | 53925148 | + | 14 | C-209 | 209 | 839 | 1469 |
| chr6 | 97282558 | 97282571 | − | 13 | C-210 | 210 | 840 | 1470 |
| chr6 | 101396242 | 101396254 | + | 12 | C-211 | 211 | 841 | 1471 |
| chr6 | 106716510 | 106716522 | + | 12 | C-212 | 212 | 842 | 1472 |
| chr6 | 109185922 | 109185935 | + | 13 | C-213 | 213 | 843 | 1473 |
| chr6 | 121888738 | 121888750 | + | 12 | C-214 | 214 | 844 | 1474 |
| chr6 | 136707557 | 136707569 | − | 12 | C-215 | 215 | 845 | 1475 |
| chr6 | 150612045 | 150612060 | + | 15 | C-216 | 216 | 846 | 1476 |
| chr6 | 163416054 | 163416068 | − | 14 | C-217 | 217 | 847 | 1477 |
| chr6 | 163963195 | 163963207 | − | 12 | C-218 | 218 | 848 | 1478 |
| chr6 | 165855305 | 165855319 | − | 14 | C-219 | 219 | 849 | 1479 |
| chr6 | 168274084 | 168274101 | − | 17 | C-220 | 220 | 850 | 1480 |
| chr7 | 516040 | 516052 | + | 12 | C-221 | 221 | 851 | 1481 |
| chr7 | 1413974 | 1413989 | + | 15 | C-222 | 222 | 852 | 1482 |
| chr7 | 1464760 | 1464773 | + | 13 | C-223 | 223 | 853 | 1483 |
| chr7 | 1814960 | 1814975 | − | 15 | C-224 | 224 | 854 | 1484 |
| chr7 | 2504914 | 2504932 | + | 18 | C-225 | 225 | 855 | 1485 |
| chr7 | 15097104 | 15097118 | + | 14 | C-226 | 226 | 856 | 1486 |
| chr7 | 19969968 | 19969983 | + | 15 | C-227 | 227 | 857 | 1487 |
| chr7 | 23202662 | 23202676 | + | 14 | C-228 | 228 | 858 | 1488 |
| chr7 | 23600374 | 23600387 | + | 13 | C-229 | 229 | 859 | 1489 |
| chr7 | 29293362 | 29293377 | + | 15 | C-230 | 230 | 860 | 1490 |
| chr7 | 30315727 | 30315740 | − | 13 | C-231 | 231 | 861 | 1491 |
| chr7 | 31098703 | 31098718 | − | 15 | C-232 | 232 | 862 | 1492 |
| chr7 | 32065051 | 32065064 | − | 13 | C-233 | 233 | 863 | 1493 |
| chr7 | 42210338 | 42210354 | + | 16 | C-234 | 234 | 864 | 1494 |
| chr7 | 55497554 | 55497570 | − | 16 | C-235 | 235 | 865 | 1495 |
| chr7 | 66628736 | 66628754 | + | 18 | C-236 | 236 | 866 | 1496 |
| chr7 | 73842642 | 73842656 | + | 14 | C-237 | 237 | 867 | 1497 |
| chr7 | 74372402 | 74372415 | − | 13 | C-238 | 238 | 868 | 1498 |
| chr7 | 76048981 | 76048998 | − | 17 | C-239 | 239 | 869 | 1499 |
| chr7 | 97004938 | 97004950 | + | 12 | C-240 | 240 | 870 | 1500 |
| chr7 | 106101438 | 106101467 | + | 29 | C-241 | 241 | 871 | 1501 |
| chr7 | 107563544 | 107563558 | − | 14 | C-242 | 242 | 872 | 1502 |
| chr7 | 112449826 | 112449840 | − | 14 | C-243 | 243 | 873 | 1503 |
| chr7 | 115477645 | 115477658 | − | 13 | C-244 | 244 | 874 | 1504 |
| chr7 | 121390853 | 121390872 | − | 19 | C-245 | 245 | 875 | 1505 |
| chr7 | 128834311 | 128834323 | + | 12 | C-246 | 246 | 876 | 1506 |
| chr7 | 135939576 | 135939588 | − | 12 | C-247 | 247 | 877 | 1507 |
| chr7 | 137381288 | 137381301 | + | 13 | C-248 | 248 | 878 | 1508 |
| chr7 | 139572764 | 139572781 | + | 17 | C-249 | 249 | 879 | 1509 |
| chr7 | 140260771 | 140260786 | + | 15 | C-250 | 250 | 880 | 1510 |
| chr7 | 151090236 | 151090248 | − | 12 | C-251 | 251 | 881 | 1511 |
| chr7 | 151210415 | 151210428 | − | 13 | C-252 | 252 | 882 | 1512 |
| chr7 | 155515294 | 155515308 | + | 14 | C-253 | 253 | 883 | 1513 |
| chr7 | 155815114 | 155815127 | + | 13 | C-254 | 254 | 884 | 1514 |
| chr7 | 157691067 | 157691081 | + | 14 | C-255 | 255 | 885 | 1515 |
| chr8 | 4201887 | 4201899 | − | 12 | C-256 | 256 | 886 | 1516 |
| chr8 | 10251861 | 10251873 | − | 12 | C-257 | 257 | 887 | 1517 |
| chr8 | 11268650 | 11268662 | − | 12 | C-258 | 258 | 888 | 1518 |
| chr8 | 11681492 | 11681508 | − | 16 | C-259 | 259 | 889 | 1519 |
| chr8 | 22049569 | 22049581 | + | 12 | C-260 | 260 | 890 | 1520 |
| chr8 | 23856296 | 23856309 | − | 13 | C-261 | 261 | 891 | 1521 |
| chr8 | 34075801 | 34075814 | − | 13 | C-262 | 262 | 892 | 1522 |
| chr8 | 38940650 | 38940662 | − | 12 | C-263 | 263 | 893 | 1523 |
| chr8 | 60714406 | 60714427 | + | 21 | C-264 | 264 | 894 | 1524 |
| chr8 | 67041122 | 67041134 | − | 12 | C-265 | 265 | 895 | 1525 |
| chr8 | 73627208 | 73627222 | − | 14 | C-266 | 266 | 896 | 1526 |
| chr8 | 81499569 | 81499581 | − | 12 | C-267 | 267 | 897 | 1527 |
| chr8 | 93916690 | 93916703 | − | 13 | C-268 | 268 | 898 | 1528 |
| chr8 | 100920795 | 100920808 | − | 13 | C-269 | 269 | 899 | 1529 |
| chr8 | 101109125 | 101109138 | + | 13 | C-270 | 270 | 900 | 1530 |
| chr8 | 101870897 | 101870909 | + | 12 | C-271 | 271 | 901 | 1531 |
| chr8 | 117015536 | 117015548 | + | 12 | C-272 | 272 | 902 | 1532 |
| chr8 | 122955069 | 122955082 | − | 13 | C-273 | 273 | 903 | 1533 |
| chr8 | 124611106 | 124611118 | + | 12 | C-274 | 274 | 904 | 1534 |
| chr8 | 127383955 | 127383967 | + | 12 | C-275 | 275 | 905 | 1535 |
| chr8 | 132070011 | 132070024 | − | 13 | C-276 | 276 | 906 | 1536 |
| chr8 | 133396203 | 133396218 | + | 15 | C-277 | 277 | 907 | 1537 |
| chr8 | 143189991 | 143190003 | − | 12 | C-278 | 278 | 908 | 1538 |
| chr8 | 143541824 | 143541837 | − | 13 | C-279 | 279 | 909 | 1539 |
| chr8 | 144513198 | 144513210 | − | 12 | C-280 | 280 | 910 | 1540 |
| chr9 | 2711382 | 2711394 | − | 12 | C-281 | 281 | 911 | 1541 |
| chr9 | 3962946 | 3962959 | − | 13 | C-282 | 282 | 912 | 1542 |
| chr9 | 4007897 | 4007909 | − | 12 | C-283 | 283 | 913 | 1543 |
| chr9 | 7568907 | 7568921 | + | 14 | C-284 | 284 | 914 | 1544 |
| chr9 | 16304806 | 16304818 | − | 12 | C-285 | 285 | 915 | 1545 |
| chr9 | 16847401 | 16847414 | − | 13 | C-286 | 286 | 916 | 1546 |
| chr9 | 18681697 | 18681709 | − | 12 | C-287 | 287 | 917 | 1547 |
| chr9 | 20263861 | 20263874 | + | 13 | C-288 | 288 | 918 | 1548 |
| chr9 | 33024861 | 33024877 | − | 16 | C-289 | 289 | 919 | 1549 |
| chr9 | 33935163 | 33935175 | − | 12 | C-290 | 290 | 920 | 1550 |
| chr9 | 38227059 | 38227074 | − | 15 | C-291 | 291 | 921 | 1551 |
| chr9 | 70629215 | 70629229 | − | 14 | C-292 | 292 | 922 | 1552 |
| chr9 | 91713941 | 91713954 | + | 13 | C-293 | 293 | 923 | 1553 |
| chr9 | 95569875 | 95569890 | + | 15 | C-294 | 294 | 924 | 1554 |
| chr9 | 97851939 | 97851951 | + | 12 | C-295 | 295 | 925 | 1555 |
| chr9 | 109120153 | 109120168 | − | 15 | C-296 | 296 | 926 | 1556 |
| chr9 | 109391870 | 109391883 | − | 13 | C-297 | 297 | 927 | 1557 |
| chr9 | 113883982 | 113884002 | + | 20 | C-298 | 298 | 928 | 1558 |
| chr9 | 126124205 | 126124220 | + | 15 | C-299 | 299 | 929 | 1559 |
| chr9 | 129889081 | 129889093 | − | 12 | C-300 | 300 | 930 | 1560 |
| chr9 | 131838409 | 131838421 | − | 12 | C-301 | 301 | 931 | 1561 |
| chr9 | 132481959 | 132481973 | − | 14 | C-302 | 302 | 932 | 1562 |
| chr9 | 134752419 | 134752431 | − | 12 | C-303 | 303 | 933 | 1563 |
| chr9 | 136131338 | 136131350 | − | 12 | C-304 | 304 | 934 | 1564 |
| chr9 | 138022805 | 138022817 | − | 12 | C-305 | 305 | 935 | 1565 |
| chr10 | 593485 | 593498 | + | 13 | C-306 | 306 | 936 | 1566 |
| chr10 | 3079662 | 3079674 | − | 12 | C-307 | 307 | 937 | 1567 |
| chr10 | 6200659 | 6200675 | − | 16 | C-308 | 308 | 938 | 1568 |
| chr10 | 7873617 | 7873629 | + | 12 | C-309 | 309 | 939 | 1569 |
| chr10 | 8052897 | 8052909 | − | 12 | C-310 | 310 | 940 | 1570 |
| chr10 | 11009005 | 11009019 | − | 14 | C-311 | 311 | 941 | 1571 |
| chr10 | 13689198 | 13689220 | − | 22 | C-312 | 312 | 942 | 1572 |
| chr10 | 21524591 | 21524606 | − | 15 | C-313 | 313 | 943 | 1573 |
| chr10 | 21751884 | 21751901 | − | 17 | C-314 | 314 | 944 | 1574 |
| chr10 | 48656265 | 48656278 | − | 13 | C-315 | 315 | 945 | 1575 |
| chr10 | 62981028 | 62981040 | − | 12 | C-316 | 316 | 946 | 1576 |
| chr10 | 72216480 | 72216495 | + | 15 | C-317 | 317 | 947 | 1577 |
| chr10 | 79067986 | 79067998 | − | 12 | C-318 | 318 | 948 | 1578 |
| chr10 | 96374961 | 96374977 | − | 16 | C-319 | 319 | 949 | 1579 |
| chr10 | 101844024 | 101844036 | − | 12 | C-320 | 320 | 950 | 1580 |
| chr10 | 102395101 | 102395118 | − | 17 | C-321 | 321 | 951 | 1581 |
| chr10 | 110208740 | 110208753 | + | 13 | C-322 | 322 | 952 | 1582 |
| chr10 | 123420681 | 123420694 | − | 13 | C-323 | 323 | 953 | 1583 |
| chr10 | 123576372 | 123576384 | − | 12 | C-324 | 324 | 954 | 1584 |
| chr10 | 124021236 | 124021248 | − | 12 | C-325 | 325 | 955 | 1585 |
| chr10 | 124727330 | 124727344 | − | 14 | C-326 | 326 | 956 | 1586 |
| chr10 | 125041504 | 125041518 | + | 14 | C-327 | 327 | 957 | 1587 |
| chr10 | 125161067 | 125161079 | − | 12 | C-328 | 328 | 958 | 1588 |
| chr10 | 126387889 | 126387901 | − | 12 | C-329 | 329 | 959 | 1589 |
| chr10 | 128993122 | 128993134 | + | 12 | C-330 | 330 | 960 | 1590 |
| chr10 | 129887054 | 129887067 | − | 13 | C-331 | 331 | 961 | 1591 |
| chr10 | 130786517 | 130786529 | + | 12 | C-332 | 332 | 962 | 1592 |
| chr10 | 132628463 | 132628478 | − | 15 | C-333 | 333 | 963 | 1593 |
| chr10 | 133277724 | 133277736 | − | 12 | C-334 | 334 | 964 | 1594 |
| chr11 | 1548766 | 1548778 | − | 12 | C-335 | 335 | 965 | 1595 |
| chr11 | 2138388 | 2138400 | − | 12 | C-336 | 336 | 966 | 1596 |
| chr11 | 11178731 | 11178744 | − | 13 | C-337 | 337 | 967 | 1597 |
| chr11 | 19831223 | 19831237 | − | 14 | C-338 | 338 | 968 | 1598 |
| chr11 | 27393939 | 27393954 | − | 15 | C-339 | 339 | 969 | 1599 |
| chr11 | 34182497 | 34182509 | − | 12 | C-340 | 340 | 970 | 1600 |
| chr11 | 45649450 | 45649464 | − | 14 | C-341 | 341 | 971 | 1601 |
| chr11 | 46382790 | 46382805 | + | 15 | C-342 | 342 | 972 | 1602 |
| chr11 | 46617540 | 46617552 | + | 12 | C-343 | 343 | 973 | 1603 |
| chr11 | 47400411 | 47400423 | − | 12 | C-344 | 344 | 974 | 1604 |
| chr11 | 57566947 | 57566959 | + | 12 | C-345 | 345 | 975 | 1605 |
| chr11 | 63938956 | 63938970 | − | 14 | C-346 | 346 | 976 | 1606 |
| chr11 | 64915132 | 64915144 | + | 12 | C-347 | 347 | 977 | 1607 |
| chr11 | 67415545 | 67415560 | − | 15 | C-348 | 348 | 978 | 1608 |
| chr11 | 72080266 | 72080280 | + | 14 | C-349 | 349 | 979 | 1609 |
| chr11 | 76784746 | 76784759 | − | 13 | C-350 | 350 | 980 | 1610 |
| chr11 | 93850670 | 93850683 | − | 13 | C-351 | 351 | 981 | 1611 |
| chr11 | 103051501 | 103051514 | + | 13 | C-352 | 352 | 982 | 1612 |
| chr11 | 103486639 | 103486651 | + | 12 | C-353 | 353 | 983 | 1613 |
| chr11 | 106997617 | 106997629 | + | 12 | C-354 | 354 | 984 | 1614 |
| chr11 | 112882103 | 112882115 | + | 12 | C-355 | 355 | 985 | 1615 |
| chr11 | 118972129 | 118972141 | + | 12 | C-356 | 356 | 986 | 1616 |
| chr11 | 121660406 | 121660418 | − | 12 | C-357 | 357 | 987 | 1617 |
| chr11 | 126929948 | 126929961 | + | 13 | C-358 | 358 | 988 | 1618 |
| chr11 | 128403216 | 128403230 | + | 14 | C-359 | 359 | 989 | 1619 |
| chr11 | 132347216 | 132347231 | + | 15 | C-360 | 360 | 990 | 1620 |
| chr11 | 134053142 | 134053155 | − | 13 | C-361 | 361 | 991 | 1621 |
| chr12 | 389101 | 389116 | − | 15 | C-362 | 362 | 992 | 1622 |
| chr12 | 568876 | 568889 | + | 13 | C-363 | 363 | 993 | 1623 |
| chr12 | 3247299 | 3247312 | + | 13 | C-364 | 364 | 994 | 1624 |
| chr12 | 3629848 | 3629860 | − | 12 | C-365 | 365 | 995 | 1625 |
| chr12 | 6960259 | 6960273 | − | 14 | C-366 | 366 | 996 | 1626 |
| chr12 | 7074066 | 7074078 | + | 12 | C-367 | 367 | 997 | 1627 |
| chr12 | 11689809 | 11689822 | + | 13 | C-368 | 368 | 998 | 1628 |
| chr12 | 13508327 | 13508339 | + | 12 | C-369 | 369 | 999 | 1629 |
| chr12 | 13736135 | 13736150 | − | 15 | C-370 | 370 | 1000 | 1630 |
| chr12 | 16605436 | 16605448 | + | 12 | C-371 | 371 | 1001 | 1631 |
| chr12 | 17831657 | 17831669 | + | 12 | C-372 | 372 | 1002 | 1632 |
| chr12 | 45407482 | 45407499 | + | 17 | C-373 | 373 | 1003 | 1633 |
| chr12 | 54026964 | 54026976 | − | 12 | C-374 | 374 | 1004 | 1634 |
| chr12 | 57136395 | 57136407 | + | 12 | C-375 | 375 | 1005 | 1635 |
| chr12 | 64675045 | 64675059 | + | 14 | C-376 | 376 | 1006 | 1636 |
| chr12 | 67068853 | 67068868 | + | 15 | C-377 | 377 | 1007 | 1637 |
| chr12 | 70576669 | 70576682 | − | 13 | C-378 | 378 | 1008 | 1638 |
| chr12 | 113830575 | 113830589 | − | 14 | C-379 | 379 | 1009 | 1639 |
| chr12 | 114766226 | 114766238 | + | 12 | C-380 | 380 | 1010 | 1640 |
| chr12 | 115968053 | 115968067 | + | 14 | C-381 | 381 | 1011 | 1641 |
| chr12 | 118981102 | 118981118 | + | 16 | C-382 | 382 | 1012 | 1642 |
| chr12 | 119019949 | 119019961 | + | 12 | C-383 | 383 | 1013 | 1643 |
| chr12 | 121299642 | 121299656 | + | 14 | C-384 | 384 | 1014 | 1644 |
| chr12 | 121799717 | 121799730 | − | 13 | C-385 | 385 | 1015 | 1645 |
| chr12 | 122969174 | 122969190 | + | 16 | C-386 | 386 | 1016 | 1646 |
| chr12 | 123532703 | 123532716 | − | 13 | C-387 | 387 | 1017 | 1647 |
| chr12 | 128266299 | 128266314 | − | 15 | C-388 | 388 | 1018 | 1648 |
| chr12 | 129029992 | 129030004 | + | 12 | C-389 | 389 | 1019 | 1649 |
| chr12 | 130312843 | 130312855 | + | 12 | C-390 | 390 | 1020 | 1650 |
| chr12 | 131827353 | 131827372 | − | 19 | C-391 | 391 | 1021 | 1651 |
| chr12 | 132181879 | 132181896 | + | 17 | C-392 | 392 | 1022 | 1652 |
| chr13 | 20229792 | 20229804 | + | 12 | C-393 | 393 | 1023 | 1653 |
| chr13 | 20720874 | 20720886 | + | 12 | C-394 | 394 | 1024 | 1654 |
| chr13 | 29360688 | 29360701 | + | 13 | C-395 | 395 | 1025 | 1655 |
| chr13 | 29544867 | 29544879 | − | 12 | C-396 | 396 | 1026 | 1656 |
| chr13 | 51767781 | 51767797 | − | 16 | C-397 | 397 | 1027 | 1657 |
| chr13 | 67140025 | 67140038 | + | 13 | C-398 | 398 | 1028 | 1658 |
| chr13 | 99347319 | 99347334 | + | 15 | C-399 | 399 | 1029 | 1659 |
| chr13 | 99894736 | 99894751 | − | 15 | C-400 | 400 | 1030 | 1660 |
| chr13 | 102140043 | 102140056 | + | 13 | C-401 | 401 | 1031 | 1661 |
| chr13 | 106386906 | 106386918 | − | 12 | C-402 | 402 | 1032 | 1662 |
| chr13 | 109818506 | 109818520 | − | 14 | C-403 | 403 | 1033 | 1663 |
| chr14 | 23120510 | 23120524 | − | 14 | C-404 | 404 | 1034 | 1664 |
| chr14 | 23487410 | 23487425 | − | 15 | C-405 | 405 | 1035 | 1665 |
| chr14 | 24232392 | 24232409 | + | 17 | C-406 | 406 | 1036 | 1666 |
| chr14 | 27137293 | 27137305 | − | 12 | C-407 | 407 | 1037 | 1667 |
| chr14 | 36520440 | 36520454 | + | 14 | C-408 | 408 | 1038 | 1668 |
| chr14 | 36658373 | 36658385 | − | 12 | C-409 | 409 | 1039 | 1669 |
| chr14 | 39395898 | 39395919 | − | 21 | C-410 | 410 | 1040 | 1670 |
| chr14 | 57865058 | 57865070 | + | 12 | C-411 | 411 | 1041 | 1671 |
| chr14 | 59869579 | 59869592 | − | 13 | C-412 | 412 | 1042 | 1672 |
| chr14 | 68755902 | 68755915 | + | 13 | C-413 | 413 | 1043 | 1673 |
| chr14 | 72859469 | 72859483 | + | 14 | C-414 | 414 | 1044 | 1674 |
| chr14 | 76864410 | 76864423 | − | 13 | C-415 | 415 | 1045 | 1675 |
| chr14 | 78248302 | 78248316 | + | 14 | C-416 | 416 | 1046 | 1676 |
| chr14 | 80187573 | 80187585 | − | 12 | C-417 | 417 | 1047 | 1677 |
| chr14 | 85511227 | 85511246 | − | 19 | C-418 | 418 | 1048 | 1678 |
| chr14 | 90398112 | 90398124 | − | 12 | C-419 | 419 | 1049 | 1679 |
| chr14 | 96740385 | 96740397 | + | 12 | C-420 | 420 | 1050 | 1680 |
| chr14 | 99042773 | 99042786 | + | 13 | C-421 | 421 | 1051 | 1681 |
| chr14 | 99420947 | 99420976 | − | 29 | C-422 | 422 | 1052 | 1682 |
| chr14 | 100147658 | 100147670 | + | 12 | C-423 | 423 | 1053 | 1683 |
| chr14 | 104677155 | 104677168 | − | 13 | C-424 | 424 | 1054 | 1684 |
| chr14 | 104730958 | 104730974 | − | 16 | C-425 | 425 | 1055 | 1685 |
| chr15 | 25656044 | 25656057 | − | 13 | C-426 | 426 | 1056 | 1686 |
| chr15 | 25939040 | 25939052 | + | 12 | C-427 | 427 | 1057 | 1687 |
| chr15 | 32804437 | 32804455 | − | 18 | C-428 | 428 | 1058 | 1688 |
| chr15 | 34721935 | 34721947 | + | 12 | C-429 | 429 | 1059 | 1689 |
| chr15 | 42401473 | 42401487 | + | 14 | C-430 | 430 | 1060 | 1690 |
| chr15 | 42411688 | 42411702 | − | 14 | C-431 | 431 | 1061 | 1691 |
| chr15 | 43509188 | 43509202 | − | 14 | C-432 | 432 | 1062 | 1692 |
| chr15 | 53758768 | 53758782 | − | 14 | C-433 | 433 | 1063 | 1693 |
| chr15 | 56465592 | 56465608 | + | 16 | C-434 | 434 | 1064 | 1694 |
| chr15 | 60540564 | 60540576 | + | 12 | C-435 | 435 | 1065 | 1695 |
| chr15 | 60876311 | 60876326 | − | 15 | C-436 | 436 | 1066 | 1696 |
| chr15 | 61119081 | 61119093 | − | 12 | C-437 | 437 | 1067 | 1697 |
| chr15 | 63270521 | 63270534 | − | 13 | C-438 | 438 | 1068 | 1698 |
| chr15 | 64743640 | 64743652 | − | 12 | C-439 | 439 | 1069 | 1699 |
| chr15 | 70289187 | 70289200 | + | 13 | C-440 | 440 | 1070 | 1700 |
| chr15 | 74873426 | 74873446 | + | 20 | C-441 | 441 | 1071 | 1701 |
| chr15 | 75451336 | 75451348 | + | 12 | C-442 | 442 | 1072 | 1702 |
| chr15 | 76954484 | 76954496 | − | 12 | C-443 | 443 | 1073 | 1703 |
| chr15 | 77020868 | 77020880 | − | 12 | C-444 | 444 | 1074 | 1704 |
| chr15 | 77072857 | 77072871 | + | 14 | C-445 | 445 | 1075 | 1705 |
| chr15 | 79086575 | 79086587 | + | 12 | C-446 | 446 | 1076 | 1706 |
| chr15 | 79091164 | 79091176 | + | 12 | C-447 | 447 | 1077 | 1707 |
| chr15 | 82132681 | 82132696 | − | 15 | C-448 | 448 | 1078 | 1708 |
| chr15 | 83435231 | 83435243 | − | 12 | C-449 | 449 | 1079 | 1709 |
| chr15 | 89221922 | 89221935 | + | 13 | C-450 | 450 | 1080 | 1710 |
| chr15 | 94579662 | 94579676 | − | 14 | C-451 | 451 | 1081 | 1711 |
| chr15 | 98971194 | 98971210 | − | 16 | C-452 | 452 | 1082 | 1712 |
| chr16 | 536003 | 536018 | + | 15 | C-453 | 453 | 1083 | 1713 |
| chr16 | 1271308 | 1271325 | − | 17 | C-454 | 454 | 1084 | 1714 |
| chr16 | 2087485 | 2087498 | − | 13 | C-455 | 455 | 1085 | 1715 |
| chr16 | 7718246 | 7718258 | − | 12 | C-456 | 456 | 1086 | 1716 |
| chr16 | 14355428 | 14355440 | − | 12 | C-457 | 457 | 1087 | 1717 |
| chr16 | 19590139 | 19590156 | + | 17 | C-458 | 458 | 1088 | 1718 |
| chr16 | 28973784 | 28973800 | − | 16 | C-459 | 459 | 1089 | 1719 |
| chr16 | 50562986 | 50562998 | + | 12 | C-460 | 460 | 1090 | 1720 |
| chr16 | 50858961 | 50858974 | + | 13 | C-461 | 461 | 1091 | 1721 |
| chr16 | 55479978 | 55479990 | − | 12 | C-462 | 462 | 1092 | 1722 |
| chr16 | 56945865 | 56945877 | + | 12 | C-463 | 463 | 1093 | 1723 |
| chr16 | 66021101 | 66021113 | + | 12 | C-464 | 464 | 1094 | 1724 |
| chr16 | 68085195 | 68085212 | − | 17 | C-465 | 465 | 1095 | 1725 |
| chr16 | 71467960 | 71467977 | − | 17 | C-466 | 466 | 1096 | 1726 |
| chr16 | 78513894 | 78513906 | + | 12 | C-467 | 467 | 1097 | 1727 |
| chr16 | 78594429 | 78594445 | + | 16 | C-468 | 468 | 1098 | 1728 |
| chr16 | 79587869 | 79587881 | − | 12 | C-469 | 469 | 1099 | 1729 |
| chr16 | 82700955 | 82700974 | + | 19 | C-470 | 470 | 1100 | 1730 |
| chr16 | 83027102 | 83027114 | + | 12 | C-471 | 471 | 1101 | 1731 |
| chr16 | 83131182 | 83131201 | + | 19 | C-472 | 472 | 1102 | 1732 |
| chr16 | 85253069 | 85253082 | + | 13 | C-473 | 473 | 1103 | 1733 |
| chr16 | 85312204 | 85312217 | − | 13 | C-474 | 474 | 1104 | 1734 |
| chr16 | 85617596 | 85617611 | + | 15 | C-475 | 475 | 1105 | 1735 |
| chr16 | 87562248 | 87562263 | + | 15 | C-476 | 476 | 1106 | 1736 |
| chr16 | 87819632 | 87819646 | − | 14 | C-477 | 477 | 1107 | 1737 |
| chr16 | 88748488 | 88748501 | + | 13 | C-478 | 478 | 1108 | 1738 |
| chr16 | 89973740 | 89973753 | + | 13 | C-479 | 479 | 1109 | 1739 |
| chr17 | 3037364 | 3037377 | + | 13 | C-480 | 480 | 1110 | 1740 |
| chr17 | 6451873 | 6451885 | + | 12 | C-481 | 481 | 1111 | 1741 |
| chr17 | 7238285 | 7238298 | + | 13 | C-482 | 482 | 1112 | 1742 |
| chr17 | 13786626 | 13786639 | − | 13 | C-483 | 483 | 1113 | 1743 |
| chr17 | 18244722 | 18244734 | − | 12 | C-484 | 484 | 1114 | 1744 |
| chr17 | 19340033 | 19340048 | + | 15 | C-485 | 485 | 1115 | 1745 |
| chr17 | 28813022 | 28813041 | − | 19 | C-486 | 486 | 1116 | 1746 |
| chr17 | 31536037 | 31536049 | − | 12 | C-487 | 487 | 1117 | 1747 |
| chr17 | 34623401 | 34623416 | + | 15 | C-488 | 488 | 1118 | 1748 |
| chr17 | 37094572 | 37094585 | + | 13 | C-489 | 489 | 1119 | 1749 |
| chr17 | 38564354 | 38564366 | + | 12 | C-490 | 490 | 1120 | 1750 |
| chr17 | 41499339 | 41499352 | + | 13 | C-491 | 491 | 1121 | 1751 |
| chr17 | 43021606 | 43021624 | − | 18 | C-492 | 492 | 1122 | 1752 |
| chr17 | 43025792 | 43025811 | − | 19 | C-493 | 493 | 1123 | 1753 |
| chr17 | 44314674 | 44314688 | − | 14 | C-494 | 494 | 1124 | 1754 |
| chr17 | 46152582 | 46152594 | − | 12 | C-495 | 495 | 1125 | 1755 |
| chr17 | 47780827 | 47780839 | − | 12 | C-496 | 496 | 1126 | 1756 |
| chr17 | 57682317 | 57682329 | + | 12 | C-497 | 497 | 1127 | 1757 |
| chr17 | 58692125 | 58692137 | − | 12 | C-498 | 498 | 1128 | 1758 |
| chr17 | 60136692 | 60136708 | + | 16 | C-499 | 499 | 1129 | 1759 |
| chr17 | 63704620 | 63704632 | − | 12 | C-500 | 500 | 1130 | 1760 |
| chr17 | 65304579 | 65304596 | + | 17 | C-501 | 501 | 1131 | 1761 |
| chr17 | 66992160 | 66992172 | − | 12 | C-502 | 502 | 1132 | 1762 |
| chr17 | 67572468 | 67572480 | − | 12 | C-503 | 503 | 1133 | 1763 |
| chr17 | 72119644 | 72119658 | − | 14 | C-504 | 504 | 1134 | 1764 |
| chr17 | 73466715 | 73466727 | + | 12 | C-505 | 505 | 1135 | 1765 |
| chr17 | 74840426 | 74840445 | + | 19 | C-506 | 506 | 1136 | 1766 |
| chr17 | 75630841 | 75630853 | − | 12 | C-507 | 507 | 1137 | 1767 |
| chr17 | 75784994 | 75785007 | + | 13 | C-508 | 508 | 1138 | 1768 |
| chr17 | 75920830 | 75920844 | − | 14 | C-509 | 509 | 1139 | 1769 |
| chr17 | 76077966 | 76077980 | − | 14 | C-510 | 510 | 1140 | 1770 |
| chr17 | 76470307 | 76470319 | − | 12 | C-511 | 511 | 1141 | 1771 |
| chr17 | 78039309 | 78039321 | + | 12 | C-512 | 512 | 1142 | 1772 |
| chr17 | 80396734 | 80396746 | + | 12 | C-513 | 513 | 1143 | 1773 |
| chr17 | 81923262 | 81923275 | + | 13 | C-514 | 514 | 1144 | 1774 |
| chr17 | 82092678 | 82092690 | − | 12 | C-515 | 515 | 1145 | 1775 |
| chr17 | 82242212 | 82242224 | − | 12 | C-516 | 516 | 1146 | 1776 |
| chr18 | 8540546 | 8540558 | − | 12 | C-517 | 517 | 1147 | 1777 |
| chr18 | 10475793 | 10475805 | − | 12 | C-518 | 518 | 1148 | 1778 |
| chr18 | 22087515 | 22087527 | − | 12 | C-519 | 519 | 1149 | 1779 |
| chr18 | 26862996 | 26863008 | − | 12 | C-520 | 520 | 1150 | 1780 |
| chr18 | 26863994 | 26864006 | − | 12 | C-521 | 521 | 1151 | 1781 |
| chr18 | 37318630 | 37318642 | + | 12 | C-522 | 522 | 1152 | 1782 |
| chr18 | 38709831 | 38709844 | + | 13 | C-523 | 523 | 1153 | 1783 |
| chr18 | 47520369 | 47520383 | + | 14 | C-524 | 524 | 1154 | 1784 |
| chr18 | 48947892 | 48947904 | + | 12 | C-525 | 525 | 1155 | 1785 |
| chr18 | 59267631 | 59267645 | + | 14 | C-526 | 526 | 1156 | 1786 |
| chr18 | 71130833 | 71130847 | + | 14 | C-527 | 527 | 1157 | 1787 |
| chr18 | 77271246 | 77271259 | + | 13 | C-528 | 528 | 1158 | 1788 |
| chr18 | 79642295 | 79642307 | + | 12 | C-529 | 529 | 1159 | 1789 |
| chr19 | 461586 | 461598 | − | 12 | C-530 | 530 | 1160 | 1790 |
| chr19 | 1605502 | 1605514 | − | 12 | C-531 | 531 | 1161 | 1791 |
| chr19 | 1610924 | 1610938 | − | 14 | C-532 | 532 | 1162 | 1792 |
| chr19 | 1648817 | 1648831 | − | 14 | C-533 | 533 | 1163 | 1793 |
| chr19 | 2188480 | 2188494 | + | 14 | C-534 | 534 | 1164 | 1794 |
| chr19 | 3061886 | 3061898 | − | 12 | C-535 | 535 | 1165 | 1795 |
| chr19 | 3722856 | 3722868 | − | 12 | C-536 | 536 | 1166 | 1796 |
| chr19 | 7679189 | 7679201 | + | 12 | C-537 | 537 | 1167 | 1797 |
| chr19 | 10419226 | 10419238 | − | 12 | C-538 | 538 | 1168 | 1798 |
| chr19 | 10457431 | 10457446 | − | 15 | C-539 | 539 | 1169 | 1799 |
| chr19 | 11192783 | 11192795 | + | 12 | C-540 | 540 | 1170 | 1800 |
| chr19 | 12944548 | 12944562 | − | 14 | C-541 | 541 | 1171 | 1801 |
| chr19 | 13018335 | 13018351 | − | 16 | C-542 | 542 | 1172 | 1802 |
| chr19 | 13226253 | 13226270 | − | 17 | C-543 | 543 | 1173 | 1803 |
| chr19 | 15251438 | 15251455 | − | 17 | C-544 | 544 | 1174 | 1804 |
| chr19 | 16873522 | 16873534 | + | 12 | C-545 | 545 | 1175 | 1805 |
| chr19 | 17686298 | 17686317 | + | 19 | C-546 | 546 | 1176 | 1806 |
| chr19 | 19213505 | 19213518 | − | 13 | C-547 | 547 | 1177 | 1807 |
| chr19 | 19277251 | 19277263 | − | 12 | C-548 | 548 | 1178 | 1808 |
| chr19 | 29269931 | 29269944 | + | 13 | C-549 | 549 | 1179 | 1809 |
| chr19 | 31959883 | 31959895 | − | 12 | C-550 | 550 | 1180 | 1810 |
| chr19 | 35030227 | 35030240 | − | 13 | C-551 | 551 | 1181 | 1811 |
| chr19 | 35900074 | 35900092 | + | 18 | C-552 | 552 | 1182 | 1812 |
| chr19 | 41253726 | 41253738 | + | 12 | C-553 | 553 | 1183 | 1813 |
| chr19 | 42370843 | 42370872 | − | 29 | C-554 | 554 | 1184 | 1814 |
| chr19 | 44749284 | 44749297 | − | 13 | C-555 | 555 | 1185 | 1815 |
| chr19 | 44753828 | 44753840 | − | 12 | C-556 | 556 | 1186 | 1816 |
| chr19 | 44759707 | 44759719 | + | 12 | C-557 | 557 | 1187 | 1817 |
| chr19 | 45450887 | 45450905 | + | 18 | C-558 | 558 | 1188 | 1818 |
| chr19 | 46408386 | 46408399 | + | 13 | C-559 | 559 | 1189 | 1819 |
| chr19 | 47020894 | 47020907 | + | 13 | C-560 | 560 | 1190 | 1820 |
| chr19 | 47531232 | 47531249 | − | 17 | C-561 | 561 | 1191 | 1821 |
| chr19 | 48638374 | 48638387 | − | 13 | C-562 | 562 | 1192 | 1822 |
| chr19 | 50427858 | 50427873 | + | 15 | C-563 | 563 | 1193 | 1823 |
| chr19 | 53962869 | 53962882 | − | 13 | C-564 | 564 | 1194 | 1824 |
| chr20 | 4721439 | 4721451 | + | 12 | C-565 | 565 | 1195 | 1825 |
| chr20 | 13280391 | 13280403 | + | 12 | C-566 | 566 | 1196 | 1826 |
| chr20 | 17990410 | 17990423 | − | 13 | C-567 | 567 | 1197 | 1827 |
| chr20 | 20021038 | 20021052 | − | 14 | C-568 | 568 | 1198 | 1828 |
| chr20 | 23421267 | 23421279 | + | 12 | C-569 | 569 | 1199 | 1829 |
| chr20 | 25390477 | 25390493 | + | 16 | C-570 | 570 | 1200 | 1830 |
| chr20 | 25696087 | 25696101 | + | 14 | C-571 | 571 | 1201 | 1831 |
| chr20 | 32536799 | 32536812 | − | 13 | C-572 | 572 | 1202 | 1832 |
| chr20 | 35398753 | 35398769 | − | 16 | C-573 | 573 | 1203 | 1833 |
| chr20 | 36269213 | 36269225 | − | 12 | C-574 | 574 | 1204 | 1834 |
| chr20 | 36744967 | 36744980 | − | 13 | C-575 | 575 | 1205 | 1835 |
| chr20 | 43188749 | 43188762 | − | 13 | C-576 | 576 | 1206 | 1836 |
| chr20 | 44434518 | 44434531 | − | 13 | C-577 | 577 | 1207 | 1837 |
| chr20 | 47057510 | 47057522 | + | 12 | C-578 | 578 | 1208 | 1838 |
| chr20 | 50155810 | 50155823 | − | 13 | C-579 | 579 | 1209 | 1839 |
| chr20 | 59867105 | 59867119 | − | 14 | C-580 | 580 | 1210 | 1840 |
| chr20 | 60589570 | 60589585 | − | 15 | C-581 | 581 | 1211 | 1841 |
| chr20 | 61797083 | 61797096 | + | 13 | C-582 | 582 | 1212 | 1842 |
| chr20 | 63274311 | 63274328 | + | 17 | C-583 | 583 | 1213 | 1843 |
| chr20 | 63774055 | 63774072 | + | 17 | C-584 | 584 | 1214 | 1844 |
| chr20 | 63940064 | 63940076 | − | 12 | C-585 | 585 | 1215 | 1845 |
| chr21 | 15733131 | 15733148 | + | 17 | C-586 | 586 | 1216 | 1846 |
| chr21 | 15775739 | 15775754 | + | 15 | C-587 | 587 | 1217 | 1847 |
| chr21 | 16071569 | 16071586 | + | 17 | C-588 | 588 | 1218 | 1848 |
| chr21 | 16373689 | 16373701 | − | 12 | C-589 | 589 | 1219 | 1849 |
| chr21 | 30322402 | 30322415 | − | 13 | C-590 | 590 | 1220 | 1850 |
| chr21 | 37476818 | 37476830 | + | 12 | C-591 | 591 | 1221 | 1851 |
| chr21 | 39349262 | 39349278 | + | 16 | C-592 | 592 | 1222 | 1852 |
| chr21 | 42405983 | 42405997 | − | 14 | C-593 | 593 | 1223 | 1853 |
| chr21 | 44538422 | 44538436 | + | 14 | C-594 | 594 | 1224 | 1854 |
| chr21 | 45750395 | 45750411 | + | 16 | C-595 | 595 | 1225 | 1855 |
| chr21 | 46436470 | 46436488 | + | 18 | C-596 | 596 | 1226 | 1856 |
| chr22 | 17159286 | 17159298 | + | 12 | C-597 | 597 | 1227 | 1857 |
| chr22 | 20111706 | 20111722 | + | 16 | C-598 | 598 | 1228 | 1858 |
| chr22 | 20971966 | 20971978 | − | 12 | C-599 | 599 | 1229 | 1859 |
| chr22 | 24425337 | 24425351 | + | 14 | C-600 | 600 | 1230 | 1860 |
| chr22 | 31291469 | 31291481 | + | 12 | C-601 | 601 | 1231 | 1861 |
| chr22 | 36348841 | 36348853 | + | 12 | C-602 | 602 | 1232 | 1862 |
| chr22 | 37641098 | 37641111 | + | 13 | C-603 | 603 | 1233 | 1863 |
| chr22 | 40819412 | 40819425 | − | 13 | C-604 | 604 | 1234 | 1864 |
| chr22 | 44602532 | 44602544 | + | 12 | C-605 | 605 | 1235 | 1865 |
| chr22 | 45122119 | 45122131 | + | 12 | C-606 | 606 | 1236 | 1866 |
| chr22 | 45181135 | 45181149 | + | 14 | C-607 | 607 | 1237 | 1867 |
| chr22 | 49648856 | 49648869 | + | 13 | C-608 | 608 | 1238 | 1868 |
| chr22 | 49787299 | 49787312 | + | 13 | C-609 | 609 | 1239 | 1869 |
| chr22 | 50185743 | 50185760 | − | 17 | C-610 | 610 | 1240 | 1870 |
| chr22 | 50261995 | 50262013 | + | 18 | C-611 | 611 | 1241 | 1871 |
| chr22 | 50446356 | 50446371 | + | 15 | C-612 | 612 | 1242 | 1872 |
| chrX | 5654055 | 5654067 | + | 12 | C-613 | 613 | 1243 | 1873 |
| chrX | 13837710 | 13837728 | − | 18 | C-614 | 614 | 1244 | 1874 |
| chrX | 17522493 | 17522506 | + | 13 | C-615 | 615 | 1245 | 1875 |
| chrX | 17737422 | 17737448 | + | 26 | C-616 | 616 | 1246 | 1876 |
| chrX | 24742159 | 24742171 | + | 12 | C-617 | 617 | 1247 | 1877 |
| chrX | 31265783 | 31265797 | − | 14 | C-618 | 618 | 1248 | 1878 |
| chrX | 32201469 | 32201483 | + | 14 | C-619 | 619 | 1249 | 1879 |
| chrX | 39878399 | 39878411 | − | 12 | C-620 | 620 | 1250 | 1880 |
| chrX | 40066921 | 40066934 | + | 13 | C-621 | 621 | 1251 | 1881 |
| chrX | 47561969 | 47561981 | + | 12 | C-622 | 622 | 1252 | 1882 |
| chrX | 48921964 | 48921977 | + | 13 | C-623 | 623 | 1253 | 1883 |
| chrX | 94343760 | 94343774 | + | 14 | C-624 | 624 | 1254 | 1884 |
| chrX | 104797185 | 104797201 | + | 16 | C-625 | 625 | 1255 | 1885 |
| chrX | 123961299 | 123961311 | + | 12 | C-626 | 626 | 1256 | 1886 |
| chrX | 129983426 | 129983438 | − | 12 | C-627 | 627 | 1257 | 1887 |
| chrX | 130632364 | 130632376 | − | 12 | C-628 | 628 | 1258 | 1888 |
| chrX | 142637522 | 142637534 | + | 12 | C-629 | 629 | 1259 | 1889 |
| chrX | 150898054 | 150898072 | + | 18 | C-630 | 630 | 1260 | 1890 |

TABLE 10
List of capture probe sequences in AC panel
Genomic coordinates, sequences, and strand information are listed for
microsatellite flanking sequences of 630 loci containing dinucleotide-AC
microsatellites, used in a hybridization capture panel.

| | | | | | | 5′ | 3′ | MS |
|---|---|---|---|---|---|---|---|---|
| | start | end | | length | | flank | flank | SEQ |
| | position | position | | in | locus | SEQ ID | SEQ ID | ID |
| chr | (hg38) | (hg38) | strand | reference | id | NO | NO | NO |
| chr1 | 5273026 | 5273042 | − | 16 | AC-1 | 1891 | 2521 | 3151 |
| chr1 | 5803266 | 5803292 | + | 26 | AC-2 | 1892 | 2522 | 3152 |
| chr1 | 9406555 | 9406580 | + | 25 | AC-3 | 1893 | 2523 | 3153 |
| chr1 | 21616136 | 21616191 | + | 55 | AC-4 | 1894 | 2524 | 3154 |
| chr1 | 32894445 | 32894457 | + | 12 | AC-5 | 1895 | 2525 | 3155 |
| chr1 | 33975486 | 33975513 | + | 27 | AC-6 | 1896 | 2526 | 3156 |
| chr1 | 36249685 | 36249720 | − | 35 | AC-7 | 1897 | 2527 | 3157 |
| chr1 | 42425070 | 42425082 | + | 12 | AC-8 | 1898 | 2528 | 3158 |
| chr1 | 47652657 | 47652700 | − | 43 | AC-9 | 1899 | 2529 | 3159 |
| chr1 | 48889989 | 48890018 | − | 29 | AC-10 | 1900 | 2530 | 3160 |
| chr1 | 48973051 | 48973063 | − | 12 | AC-11 | 1901 | 2531 | 3161 |
| chr1 | 52612060 | 52612072 | + | 12 | AC-12 | 1902 | 2532 | 3162 |
| chr1 | 65166310 | 65166351 | − | 41 | AC-13 | 1903 | 2533 | 3163 |
| chr1 | 67766106 | 67766149 | + | 43 | AC-14 | 1904 | 2534 | 3164 |
| chr1 | 71887992 | 71888036 | + | 44 | AC-15 | 1905 | 2535 | 3165 |
| chr1 | 85577477 | 85577491 | + | 14 | AC-16 | 1906 | 2536 | 3166 |
| chr1 | 85608106 | 85608118 | + | 12 | AC-17 | 1907 | 2537 | 3167 |
| chr1 | 87878048 | 87878073 | − | 25 | AC-18 | 1908 | 2538 | 3168 |
| chr1 | 91073372 | 91073384 | + | 12 | AC-19 | 1909 | 2539 | 3169 |
| chr1 | 95614000 | 95614041 | − | 41 | AC-20 | 1910 | 2540 | 3170 |
| chr1 | 99574683 | 99574697 | − | 14 | AC-21 | 1911 | 2541 | 3171 |
| chr1 | 101895412 | 101895448 | + | 36 | AC-22 | 1912 | 2542 | 3172 |
| chr1 | 113129531 | 113129551 | + | 20 | AC-23 | 1913 | 2543 | 3173 |
| chr1 | 118556180 | 118556203 | + | 23 | AC-24 | 1914 | 2544 | 3174 |
| chr1 | 152512802 | 152512839 | − | 37 | AC-25 | 1915 | 2545 | 3175 |
| chr1 | 154429844 | 154429857 | − | 13 | AC-26 | 1916 | 2546 | 3176 |
| chr1 | 155129513 | 155129548 | + | 35 | AC-27 | 1917 | 2547 | 3177 |
| chr1 | 157013263 | 157013300 | + | 37 | AC-28 | 1918 | 2548 | 3178 |
| chr1 | 160588982 | 160589019 | + | 37 | AC-29 | 1919 | 2549 | 3179 |
| chr1 | 163144212 | 163144224 | − | 12 | AC-30 | 1920 | 2550 | 3180 |
| chr1 | 165928155 | 165928186 | − | 31 | AC-31 | 1921 | 2551 | 3181 |
| chr1 | 168317787 | 168317810 | − | 23 | AC-32 | 1922 | 2552 | 3182 |
| chr1 | 168398963 | 168399004 | − | 41 | AC-33 | 1923 | 2553 | 3183 |
| chr1 | 169369080 | 169369093 | − | 13 | AC-34 | 1924 | 2554 | 3184 |
| chr1 | 176331456 | 176331506 | − | 50 | AC-35 | 1925 | 2555 | 3185 |
| chr1 | 181356341 | 181356363 | − | 22 | AC-36 | 1926 | 2556 | 3186 |
| chr1 | 182289911 | 182289962 | + | 51 | AC-37 | 1927 | 2557 | 3187 |
| chr1 | 182644335 | 182644347 | + | 12 | AC-38 | 1928 | 2558 | 3188 |
| chr1 | 185158268 | 185158282 | − | 14 | AC-39 | 1929 | 2559 | 3189 |
| chr1 | 186375993 | 186376022 | − | 29 | AC-40 | 1930 | 2560 | 3190 |
| chr1 | 189039446 | 189039461 | + | 15 | AC-41 | 1931 | 2561 | 3191 |
| chr1 | 203102658 | 203102694 | + | 36 | AC-42 | 1932 | 2562 | 3192 |
| chr1 | 203997182 | 203997230 | − | 48 | AC-43 | 1933 | 2563 | 3193 |
| chr1 | 205048910 | 205048960 | − | 50 | AC-44 | 1934 | 2564 | 3194 |
| chr1 | 206501638 | 206501680 | + | 42 | AC-45 | 1935 | 2565 | 3195 |
| chr1 | 214214171 | 214214218 | + | 47 | AC-46 | 1936 | 2566 | 3196 |
| chr1 | 221277846 | 221277859 | + | 13 | AC-47 | 1937 | 2567 | 3197 |
| chr1 | 222424829 | 222424841 | + | 12 | AC-48 | 1938 | 2568 | 3198 |
| chr1 | 223036108 | 223036121 | − | 13 | AC-49 | 1939 | 2569 | 3199 |
| chr1 | 230353426 | 230353440 | + | 14 | AC-50 | 1940 | 2570 | 3200 |
| chr1 | 232060618 | 232060630 | + | 12 | AC-51 | 1941 | 2571 | 3201 |
| chr1 | 233512134 | 233512149 | − | 15 | AC-52 | 1942 | 2572 | 3202 |
| chr1 | 238887748 | 238887773 | + | 25 | AC-53 | 1943 | 2573 | 3203 |
| chr1 | 241147137 | 241147149 | + | 12 | AC-54 | 1944 | 2574 | 3204 |
| chr10 | 1994136 | 1994171 | + | 35 | AC-55 | 1945 | 2575 | 3205 |
| chr10 | 3343104 | 3343138 | + | 34 | AC-56 | 1946 | 2576 | 3206 |
| chr10 | 7085186 | 7085199 | + | 13 | AC-57 | 1947 | 2577 | 3207 |
| chr10 | 7572113 | 7572126 | − | 13 | AC-58 | 1948 | 2578 | 3208 |
| chr10 | 12704059 | 12704075 | − | 16 | AC-59 | 1949 | 2579 | 3209 |
| chr10 | 16661287 | 16661329 | − | 42 | AC-60 | 1950 | 2580 | 3210 |
| chr10 | 23292998 | 23293016 | − | 18 | AC-61 | 1951 | 2581 | 3211 |
| chr10 | 23691684 | 23691737 | − | 53 | AC-62 | 1952 | 2582 | 3212 |
| chr10 | 31073663 | 31073703 | − | 40 | AC-63 | 1953 | 2583 | 3213 |
| chr10 | 43143493 | 43143537 | − | 44 | AC-64 | 1954 | 2584 | 3214 |
| chr10 | 58988933 | 58988954 | − | 21 | AC-65 | 1955 | 2585 | 3215 |
| chr10 | 60272089 | 60272127 | − | 38 | AC-66 | 1956 | 2586 | 3216 |
| chr10 | 61238230 | 61238242 | + | 12 | AC-67 | 1957 | 2587 | 3217 |
| chr10 | 61993120 | 61993150 | + | 30 | AC-68 | 1958 | 2588 | 3218 |
| chr10 | 62563328 | 62563363 | + | 35 | AC-69 | 1959 | 2589 | 3219 |
| chr10 | 69735131 | 69735143 | + | 12 | AC-70 | 1960 | 2590 | 3220 |
| chr10 | 85616976 | 85616988 | + | 12 | AC-71 | 1961 | 2591 | 3221 |
| chr10 | 87703276 | 87703316 | − | 40 | AC-72 | 1962 | 2592 | 3222 |
| chr10 | 89086320 | 89086358 | − | 38 | AC-73 | 1963 | 2593 | 3223 |
| chr10 | 89172566 | 89172602 | − | 36 | AC-74 | 1964 | 2594 | 3224 |
| chr10 | 101249577 | 101249590 | − | 13 | AC-75 | 1965 | 2595 | 3225 |
| chr10 | 101566446 | 101566458 | − | 12 | AC-76 | 1966 | 2596 | 3226 |
| chr10 | 113621314 | 113621341 | + | 27 | AC-77 | 1967 | 2597 | 3227 |
| chr10 | 113632834 | 113632848 | − | 14 | AC-78 | 1968 | 2598 | 3228 |
| chr10 | 125841187 | 125841200 | − | 13 | AC-79 | 1969 | 2599 | 3229 |
| chr11 | 2834067 | 2834079 | + | 12 | AC-80 | 1970 | 2600 | 3230 |
| chr11 | 10900297 | 10900333 | − | 36 | AC-81 | 1971 | 2601 | 3231 |
| chr11 | 19953858 | 19953882 | − | 24 | AC-82 | 1972 | 2602 | 3232 |
| chr11 | 23347472 | 23347508 | + | 36 | AC-83 | 1973 | 2603 | 3233 |
| chr11 | 23447836 | 23447867 | − | 31 | AC-84 | 1974 | 2604 | 3234 |
| chr11 | 25680078 | 25680113 | + | 35 | AC-85 | 1975 | 2605 | 3235 |
| chr11 | 38474448 | 38474491 | + | 43 | AC-86 | 1976 | 2606 | 3236 |
| chr11 | 39114247 | 39114284 | − | 37 | AC-87 | 1977 | 2607 | 3237 |
| chr11 | 56754460 | 56754476 | + | 16 | AC-88 | 1978 | 2608 | 3238 |
| chr11 | 58689177 | 58689190 | + | 13 | AC-89 | 1979 | 2609 | 3239 |
| chr11 | 70660519 | 70660531 | − | 12 | AC-90 | 1980 | 2610 | 3240 |
| chr11 | 77304988 | 77305031 | + | 43 | AC-91 | 1981 | 2611 | 3241 |
| chr11 | 79081534 | 79081561 | − | 27 | AC-92 | 1982 | 2612 | 3242 |
| chr11 | 87376057 | 87376087 | + | 30 | AC-93 | 1983 | 2613 | 3243 |
| chr11 | 89491727 | 89491766 | + | 39 | AC-94 | 1984 | 2614 | 3244 |
| chr11 | 93633463 | 93633502 | + | 39 | AC-95 | 1985 | 2615 | 3245 |
| chr11 | 105917815 | 105917855 | − | 40 | AC-96 | 1986 | 2616 | 3246 |
| chr11 | 106990585 | 106990601 | − | 16 | AC-97 | 1987 | 2617 | 3247 |
| chr11 | 109832817 | 109832838 | − | 21 | AC-98 | 1988 | 2618 | 3248 |
| chr11 | 110953986 | 110953999 | + | 13 | AC-99 | 1989 | 2619 | 3249 |
| chr11 | 116660717 | 116660733 | + | 16 | AC-100 | 1990 | 2620 | 3250 |
| chr11 | 117738113 | 117738126 | − | 13 | AC-101 | 1991 | 2621 | 3251 |
| chr11 | 121235552 | 121235584 | + | 32 | AC-102 | 1992 | 2622 | 3252 |
| chr11 | 125612783 | 125612798 | − | 15 | AC-103 | 1993 | 2623 | 3253 |
| chr11 | 125748814 | 125748835 | + | 21 | AC-104 | 1994 | 2624 | 3254 |
| chr11 | 127148450 | 127148470 | − | 20 | AC-105 | 1995 | 2625 | 3255 |
| chr12 | 152701 | 152713 | + | 12 | AC-106 | 1996 | 2626 | 3256 |
| chr12 | 4166085 | 4166098 | + | 13 | AC-107 | 1997 | 2627 | 3257 |
| chr12 | 8751503 | 8751515 | − | 12 | AC-108 | 1998 | 2628 | 3258 |
| chr12 | 10223341 | 10223370 | − | 29 | AC-109 | 1999 | 2629 | 3259 |
| chr12 | 13085665 | 13085678 | + | 13 | AC-110 | 2000 | 2630 | 3260 |
| chr12 | 26451365 | 26451408 | + | 43 | AC-111 | 2001 | 2631 | 3261 |
| chr12 | 27909439 | 27909474 | − | 35 | AC-112 | 2002 | 2632 | 3262 |
| chr12 | 31340560 | 31340572 | − | 12 | AC-113 | 2003 | 2633 | 3263 |
| chr12 | 43288359 | 43288378 | − | 19 | AC-114 | 2004 | 2634 | 3264 |
| chr12 | 55318221 | 55318264 | − | 43 | AC-115 | 2005 | 2635 | 3265 |
| chr12 | 56190258 | 56190270 | − | 12 | AC-116 | 2006 | 2636 | 3266 |
| chr12 | 57139812 | 57139825 | + | 13 | AC-117 | 2007 | 2637 | 3267 |
| chr12 | 58815083 | 58815095 | + | 12 | AC-118 | 2008 | 2638 | 3268 |
| chr12 | 61520450 | 61520472 | + | 22 | AC-119 | 2009 | 2639 | 3269 |
| chr12 | 64948574 | 64948623 | + | 49 | AC-120 | 2010 | 2640 | 3270 |
| chr12 | 65639748 | 65639791 | + | 43 | AC-121 | 2011 | 2641 | 3271 |
| chr12 | 65653655 | 65653668 | + | 13 | AC-122 | 2012 | 2642 | 3272 |
| chr12 | 65931551 | 65931590 | − | 39 | AC-123 | 2013 | 2643 | 3273 |
| chr12 | 74155091 | 74155103 | + | 12 | AC-124 | 2014 | 2644 | 3274 |
| chr12 | 75213132 | 75213167 | + | 35 | AC-125 | 2015 | 2645 | 3275 |
| chr12 | 75544924 | 75544941 | − | 17 | AC-126 | 2016 | 2646 | 3276 |
| chr12 | 79269695 | 79269714 | + | 19 | AC-127 | 2017 | 2647 | 3277 |
| chr12 | 80039018 | 80039033 | − | 15 | AC-128 | 2018 | 2648 | 3278 |
| chr12 | 83664411 | 83664431 | − | 20 | AC-129 | 2019 | 2649 | 3279 |
| chr12 | 85465254 | 85465291 | − | 37 | AC-130 | 2020 | 2650 | 3280 |
| chr12 | 88070093 | 88070105 | + | 12 | AC-131 | 2021 | 2651 | 3281 |
| chr12 | 96236589 | 96236602 | − | 13 | AC-132 | 2022 | 2652 | 3282 |
| chr12 | 96521503 | 96521548 | − | 45 | AC-133 | 2023 | 2653 | 3283 |
| chr12 | 105427778 | 105427802 | − | 24 | AC-134 | 2024 | 2654 | 3284 |
| chr12 | 108130475 | 108130521 | − | 46 | AC-135 | 2025 | 2655 | 3285 |
| chr12 | 113048989 | 113049024 | + | 35 | AC-136 | 2026 | 2656 | 3286 |
| chr12 | 114001557 | 114001574 | − | 17 | AC-137 | 2027 | 2657 | 3287 |
| chr12 | 120766100 | 120766142 | + | 42 | AC-138 | 2028 | 2658 | 3288 |
| chr12 | 127558627 | 127558642 | − | 15 | AC-139 | 2029 | 2659 | 3289 |
| chr13 | 25139859 | 25139888 | + | 29 | AC-140 | 2030 | 2660 | 3290 |
| chr13 | 26944328 | 26944373 | − | 45 | AC-141 | 2031 | 2661 | 3291 |
| chr13 | 27923032 | 27923044 | + | 12 | AC-142 | 2032 | 2662 | 3292 |
| chr13 | 37492152 | 37492172 | − | 20 | AC-143 | 2033 | 2663 | 3293 |
| chr13 | 40235907 | 40235949 | − | 42 | AC-144 | 2034 | 2664 | 3294 |
| chr13 | 47623464 | 47623478 | + | 14 | AC-145 | 2035 | 2665 | 3295 |
| chr13 | 50727214 | 50727233 | − | 19 | AC-146 | 2036 | 2666 | 3296 |
| chr13 | 62203068 | 62203085 | − | 17 | AC-147 | 2037 | 2667 | 3297 |
| chr13 | 66659804 | 66659817 | − | 13 | AC-148 | 2038 | 2668 | 3298 |
| chr13 | 70313232 | 70313246 | + | 14 | AC-149 | 2039 | 2669 | 3299 |
| chr13 | 71216982 | 71217004 | − | 22 | AC-150 | 2040 | 2670 | 3300 |
| chr13 | 84316395 | 84316410 | + | 15 | AC-151 | 2041 | 2671 | 3301 |
| chr13 | 94796168 | 94796210 | − | 42 | AC-152 | 2042 | 2672 | 3302 |
| chr13 | 96868406 | 96868423 | + | 17 | AC-153 | 2043 | 2673 | 3303 |
| chr13 | 101347140 | 101347166 | + | 26 | AC-154 | 2044 | 2674 | 3304 |
| chr13 | 101400544 | 101400581 | − | 37 | AC-155 | 2045 | 2675 | 3305 |
| chr13 | 104766495 | 104766509 | + | 14 | AC-156 | 2046 | 2676 | 3306 |
| chr13 | 106836459 | 106836477 | + | 18 | AC-157 | 2047 | 2677 | 3307 |
| chr14 | 21067761 | 21067785 | + | 24 | AC-158 | 2048 | 2678 | 3308 |
| chr14 | 38003713 | 38003727 | − | 14 | AC-159 | 2049 | 2679 | 3309 |
| chr14 | 41404996 | 41405011 | + | 15 | AC-160 | 2050 | 2680 | 3310 |
| chr14 | 44327286 | 44327298 | + | 12 | AC-161 | 2051 | 2681 | 3311 |
| chr14 | 51294075 | 51294088 | − | 13 | AC-162 | 2052 | 2682 | 3312 |
| chr14 | 55921918 | 55921931 | − | 13 | AC-163 | 2053 | 2683 | 3313 |
| chr14 | 56583990 | 56584027 | − | 37 | AC-164 | 2054 | 2684 | 3314 |
| chr14 | 57073832 | 57073845 | + | 13 | AC-165 | 2055 | 2685 | 3315 |
| chr14 | 57477486 | 57477505 | + | 19 | AC-166 | 2056 | 2686 | 3316 |
| chr14 | 59156718 | 59156749 | − | 31 | AC-167 | 2057 | 2687 | 3317 |
| chr14 | 67225926 | 67225963 | − | 37 | AC-168 | 2058 | 2688 | 3318 |
| chr14 | 67442428 | 67442441 | − | 13 | AC-169 | 2059 | 2689 | 3319 |
| chr14 | 67657598 | 67657623 | + | 25 | AC-170 | 2060 | 2690 | 3320 |
| chr14 | 69731578 | 69731625 | − | 47 | AC-171 | 2061 | 2691 | 3321 |
| chr14 | 84017033 | 84017047 | − | 14 | AC-172 | 2062 | 2692 | 3322 |
| chr14 | 88942813 | 88942825 | + | 12 | AC-173 | 2063 | 2693 | 3323 |
| chr14 | 97600226 | 97600239 | − | 13 | AC-174 | 2064 | 2694 | 3324 |
| chr14 | 98844420 | 98844434 | + | 14 | AC-175 | 2065 | 2695 | 3325 |
| chr14 | 99029822 | 99029834 | − | 12 | AC-176 | 2066 | 2696 | 3326 |
| chr14 | 99094458 | 99094479 | + | 21 | AC-177 | 2067 | 2697 | 3327 |
| chr14 | 99261878 | 99261895 | + | 17 | AC-178 | 2068 | 2698 | 3328 |
| chr15 | 31775089 | 31775119 | + | 30 | AC-179 | 2069 | 2699 | 3329 |
| chr15 | 36645494 | 36645532 | − | 38 | AC-180 | 2070 | 2700 | 3330 |
| chr15 | 36774133 | 36774164 | − | 31 | AC-181 | 2071 | 2701 | 3331 |
| chr15 | 40107533 | 40107575 | − | 42 | AC-182 | 2072 | 2702 | 3332 |
| chr15 | 44628870 | 44628885 | − | 15 | AC-183 | 2073 | 2703 | 3333 |
| chr15 | 47331277 | 47331289 | − | 12 | AC-184 | 2074 | 2704 | 3334 |
| chr15 | 48975543 | 48975560 | + | 17 | AC-185 | 2075 | 2705 | 3335 |
| chr15 | 48982679 | 48982732 | − | 53 | AC-186 | 2076 | 2706 | 3336 |
| chr15 | 53246231 | 53246268 | + | 37 | AC-187 | 2077 | 2707 | 3337 |
| chr15 | 58586916 | 58586950 | − | 34 | AC-188 | 2078 | 2708 | 3338 |
| chr15 | 66800703 | 66800735 | + | 32 | AC-189 | 2079 | 2709 | 3339 |
| chr15 | 71741684 | 71741728 | + | 44 | AC-190 | 2080 | 2710 | 3340 |
| chr15 | 83479331 | 83479351 | − | 20 | AC-191 | 2081 | 2711 | 3341 |
| chr15 | 88392218 | 88392257 | − | 39 | AC-192 | 2082 | 2712 | 3342 |
| chr15 | 100721147 | 100721183 | − | 36 | AC-193 | 2083 | 2713 | 3343 |
| chr16 | 4518154 | 4518169 | + | 15 | AC-194 | 2084 | 2714 | 3344 |
| chr16 | 10220039 | 10220052 | + | 13 | AC-195 | 2085 | 2715 | 3345 |
| chr16 | 13058751 | 13058783 | − | 32 | AC-196 | 2086 | 2716 | 3346 |
| chr16 | 19143314 | 19143326 | + | 12 | AC-197 | 2087 | 2717 | 3347 |
| chr16 | 20008079 | 20008123 | − | 44 | AC-198 | 2088 | 2718 | 3348 |
| chr16 | 22170787 | 22170800 | − | 13 | AC-199 | 2089 | 2719 | 3349 |
| chr16 | 22622485 | 22622529 | − | 44 | AC-200 | 2090 | 2720 | 3350 |
| chr16 | 25270696 | 25270708 | + | 12 | AC-201 | 2091 | 2721 | 3351 |
| chr16 | 31187213 | 31187225 | − | 12 | AC-202 | 2092 | 2722 | 3352 |
| chr16 | 51187020 | 51187071 | − | 51 | AC-203 | 2093 | 2723 | 3353 |
| chr16 | 54173946 | 54173958 | − | 12 | AC-204 | 2094 | 2724 | 3354 |
| chr16 | 63276767 | 63276789 | − | 22 | AC-205 | 2095 | 2725 | 3355 |
| chr16 | 64226570 | 64226585 | + | 15 | AC-206 | 2096 | 2726 | 3356 |
| chr16 | 65286227 | 65286269 | + | 42 | AC-207 | 2097 | 2727 | 3357 |
| chr16 | 66902100 | 66902137 | + | 37 | AC-208 | 2098 | 2728 | 3358 |
| chr16 | 79398635 | 79398650 | − | 15 | AC-209 | 2099 | 2729 | 3359 |
| chr16 | 80163777 | 80163800 | + | 23 | AC-210 | 2100 | 2730 | 3360 |
| chr16 | 84126736 | 84126751 | + | 15 | AC-211 | 2101 | 2731 | 3361 |
| chr16 | 85135615 | 85135636 | − | 21 | AC-212 | 2102 | 2732 | 3362 |
| chr17 | 17358350 | 17358381 | + | 31 | AC-213 | 2103 | 2733 | 3363 |
| chr17 | 33317886 | 33317928 | − | 42 | AC-214 | 2104 | 2734 | 3364 |
| chr17 | 34554923 | 34554938 | + | 15 | AC-215 | 2105 | 2735 | 3365 |
| chr17 | 39236064 | 39236095 | − | 31 | AC-216 | 2106 | 2736 | 3366 |
| chr17 | 40309936 | 40309953 | − | 17 | AC-217 | 2107 | 2737 | 3367 |
| chr17 | 49514056 | 49514070 | + | 14 | AC-218 | 2108 | 2738 | 3368 |
| chr17 | 50852653 | 50852695 | + | 42 | AC-219 | 2109 | 2739 | 3369 |
| chr17 | 51007110 | 51007139 | − | 29 | AC-220 | 2110 | 2740 | 3370 |
| chr17 | 65137980 | 65137994 | − | 14 | AC-221 | 2111 | 2741 | 3371 |
| chr17 | 65205024 | 65205036 | − | 12 | AC-222 | 2112 | 2742 | 3372 |
| chr17 | 66444942 | 66444954 | + | 12 | AC-223 | 2113 | 2743 | 3373 |
| chr17 | 69507332 | 69507356 | − | 24 | AC-224 | 2114 | 2744 | 3374 |
| chr17 | 71156495 | 71156527 | + | 32 | AC-225 | 2115 | 2745 | 3375 |
| chr17 | 76451779 | 76451803 | − | 24 | AC-226 | 2116 | 2746 | 3376 |
| chr17 | 81522657 | 81522670 | − | 13 | AC-227 | 2117 | 2747 | 3377 |
| chr18 | 5931560 | 5931583 | + | 23 | AC-228 | 2118 | 2748 | 3378 |
| chr18 | 28860767 | 28860798 | + | 31 | AC-229 | 2119 | 2749 | 3379 |
| chr18 | 31709553 | 31709604 | − | 51 | AC-230 | 2120 | 2750 | 3380 |
| chr18 | 34496205 | 34496245 | + | 40 | AC-231 | 2121 | 2751 | 3381 |
| chr18 | 34633662 | 34633674 | + | 12 | AC-232 | 2122 | 2752 | 3382 |
| chr18 | 35431510 | 35431526 | − | 16 | AC-233 | 2123 | 2753 | 3383 |
| chr18 | 38159575 | 38159613 | + | 38 | AC-234 | 2124 | 2754 | 3384 |
| chr18 | 47701045 | 47701057 | − | 12 | AC-235 | 2125 | 2755 | 3385 |
| chr18 | 47717937 | 47717966 | − | 29 | AC-236 | 2126 | 2756 | 3386 |
| chr18 | 49027460 | 49027503 | − | 43 | AC-237 | 2127 | 2757 | 3387 |
| chr18 | 55538026 | 55538067 | + | 41 | AC-238 | 2128 | 2758 | 3388 |
| chr18 | 56524136 | 56524159 | + | 23 | AC-239 | 2129 | 2759 | 3389 |
| chr18 | 60545292 | 60545339 | − | 47 | AC-240 | 2130 | 2760 | 3390 |
| chr18 | 62828008 | 62828045 | − | 37 | AC-241 | 2131 | 2761 | 3391 |
| chr18 | 64406019 | 64406053 | + | 34 | AC-242 | 2132 | 2762 | 3392 |
| chr18 | 66444246 | 66444258 | − | 12 | AC-243 | 2133 | 2763 | 3393 |
| chr18 | 67130891 | 67130908 | − | 17 | AC-244 | 2134 | 2764 | 3394 |
| chr18 | 71055956 | 71056004 | − | 48 | AC-245 | 2135 | 2765 | 3395 |
| chr18 | 72205692 | 72205707 | + | 15 | AC-246 | 2136 | 2766 | 3396 |
| chr18 | 73334311 | 73334354 | − | 43 | AC-247 | 2137 | 2767 | 3397 |
| chr18 | 74589244 | 74589259 | + | 15 | AC-248 | 2138 | 2768 | 3398 |
| chr18 | 77992959 | 77992972 | − | 13 | AC-249 | 2139 | 2769 | 3399 |
| chr19 | 1304563 | 1304575 | + | 12 | AC-250 | 2140 | 2770 | 3400 |
| chr19 | 1524600 | 1524632 | − | 32 | AC-251 | 2141 | 2771 | 3401 |
| chr19 | 13400195 | 13400208 | − | 13 | AC-252 | 2142 | 2772 | 3402 |
| chr19 | 28132636 | 28132661 | + | 25 | AC-253 | 2143 | 2773 | 3403 |
| chr19 | 33269330 | 33269364 | − | 34 | AC-254 | 2144 | 2774 | 3404 |
| chr19 | 33578137 | 33578165 | − | 28 | AC-255 | 2145 | 2775 | 3405 |
| chr19 | 33800199 | 33800211 | − | 12 | AC-256 | 2146 | 2776 | 3406 |
| chr19 | 35501212 | 35501226 | + | 14 | AC-257 | 2147 | 2777 | 3407 |
| chr19 | 40441325 | 40441338 | + | 13 | AC-258 | 2148 | 2778 | 3408 |
| chr2 | 5621363 | 5621377 | + | 14 | AC-259 | 2149 | 2779 | 3409 |
| chr2 | 6503432 | 6503463 | + | 31 | AC-260 | 2150 | 2780 | 3410 |
| chr2 | 7795345 | 7795387 | − | 42 | AC-261 | 2151 | 2781 | 3411 |
| chr2 | 8035588 | 8035600 | + | 12 | AC-262 | 2152 | 2782 | 3412 |
| chr2 | 11842276 | 11842324 | − | 48 | AC-263 | 2153 | 2783 | 3413 |
| chr2 | 12196457 | 12196469 | + | 12 | AC-264 | 2154 | 2784 | 3414 |
| chr2 | 18653522 | 18653538 | − | 16 | AC-265 | 2155 | 2785 | 3415 |
| chr2 | 19339740 | 19339753 | − | 13 | AC-266 | 2156 | 2786 | 3416 |
| chr2 | 19914533 | 19914548 | + | 15 | AC-267 | 2157 | 2787 | 3417 |
| chr2 | 26617120 | 26617148 | − | 28 | AC-268 | 2158 | 2788 | 3418 |
| chr2 | 27696661 | 27696677 | + | 16 | AC-269 | 2159 | 2789 | 3419 |
| chr2 | 42711783 | 42711815 | − | 32 | AC-270 | 2160 | 2790 | 3420 |
| chr2 | 47070401 | 47070413 | − | 12 | AC-271 | 2161 | 2791 | 3421 |
| chr2 | 50759826 | 50759870 | − | 44 | AC-272 | 2162 | 2792 | 3422 |
| chr2 | 54953457 | 54953469 | − | 12 | AC-273 | 2163 | 2793 | 3423 |
| chr2 | 55765032 | 55765044 | − | 12 | AC-274 | 2164 | 2794 | 3424 |
| chr2 | 58627465 | 58627486 | − | 21 | AC-275 | 2165 | 2795 | 3425 |
| chr2 | 61705891 | 61705903 | + | 12 | AC-276 | 2166 | 2796 | 3426 |
| chr2 | 62588519 | 62588551 | + | 32 | AC-277 | 2167 | 2797 | 3427 |
| chr2 | 66189715 | 66189727 | + | 12 | AC-278 | 2168 | 2798 | 3428 |
| chr2 | 70827642 | 70827657 | + | 15 | AC-279 | 2169 | 2799 | 3429 |
| chr2 | 70943187 | 70943207 | + | 20 | AC-280 | 2170 | 2800 | 3430 |
| chr2 | 71342683 | 71342697 | − | 14 | AC-281 | 2171 | 2801 | 3431 |
| chr2 | 75427136 | 75427178 | − | 42 | AC-282 | 2172 | 2802 | 3432 |
| chr2 | 77605188 | 77605209 | + | 21 | AC-283 | 2173 | 2803 | 3433 |
| chr2 | 80183884 | 80183910 | − | 26 | AC-284 | 2174 | 2804 | 3434 |
| chr2 | 85790322 | 85790334 | − | 12 | AC-285 | 2175 | 2805 | 3435 |
| chr2 | 95144909 | 95144926 | + | 17 | AC-286 | 2176 | 2806 | 3436 |
| chr2 | 100895107 | 100895120 | + | 13 | AC-287 | 2177 | 2807 | 3437 |
| chr2 | 103211514 | 103211529 | − | 15 | AC-288 | 2178 | 2808 | 3438 |
| chr2 | 104530832 | 104530848 | + | 16 | AC-289 | 2179 | 2809 | 3439 |
| chr2 | 105294307 | 105294337 | − | 30 | AC-290 | 2180 | 2810 | 3440 |
| chr2 | 105440122 | 105440134 | + | 12 | AC-291 | 2181 | 2811 | 3441 |
| chr2 | 117424784 | 117424797 | − | 13 | AC-292 | 2182 | 2812 | 3442 |
| chr2 | 118851472 | 118851509 | − | 37 | AC-293 | 2183 | 2813 | 3443 |
| chr2 | 121528603 | 121528615 | + | 12 | AC-294 | 2184 | 2814 | 3444 |
| chr2 | 121804439 | 121804454 | − | 15 | AC-295 | 2185 | 2815 | 3445 |
| chr2 | 122661750 | 122661762 | − | 12 | AC-296 | 2186 | 2816 | 3446 |
| chr2 | 133586751 | 133586795 | + | 44 | AC-297 | 2187 | 2817 | 3447 |
| chr2 | 137893068 | 137893081 | + | 13 | AC-298 | 2188 | 2818 | 3448 |
| chr2 | 141480159 | 141480194 | − | 35 | AC-299 | 2189 | 2819 | 3449 |
| chr2 | 141517273 | 141517289 | + | 16 | AC-300 | 2190 | 2820 | 3450 |
| chr2 | 144019435 | 144019483 | − | 48 | AC-301 | 2191 | 2821 | 3451 |
| chr2 | 144368870 | 144368887 | + | 17 | AC-302 | 2192 | 2822 | 3452 |
| chr2 | 146239369 | 146239408 | + | 39 | AC-303 | 2193 | 2823 | 3453 |
| chr2 | 147556100 | 147556138 | − | 38 | AC-304 | 2194 | 2824 | 3454 |
| chr2 | 149149417 | 149149433 | + | 16 | AC-305 | 2195 | 2825 | 3455 |
| chr2 | 167196412 | 167196451 | − | 39 | AC-306 | 2196 | 2826 | 3456 |
| chr2 | 170313865 | 170313878 | + | 13 | AC-307 | 2197 | 2827 | 3457 |
| chr2 | 170375721 | 170375745 | + | 24 | AC-308 | 2198 | 2828 | 3458 |
| chr2 | 175171165 | 175171184 | − | 19 | AC-309 | 2199 | 2829 | 3459 |
| chr2 | 177652123 | 177652135 | + | 12 | AC-310 | 2200 | 2830 | 3460 |
| chr2 | 178791663 | 178791689 | − | 26 | AC-311 | 2201 | 2831 | 3461 |
| chr2 | 180327353 | 180327365 | − | 12 | AC-312 | 2202 | 2832 | 3462 |
| chr2 | 198126991 | 198127012 | − | 21 | AC-313 | 2203 | 2833 | 3463 |
| chr2 | 199627446 | 199627478 | − | 32 | AC-314 | 2204 | 2834 | 3464 |
| chr2 | 201933089 | 201933105 | − | 16 | AC-315 | 2205 | 2835 | 3465 |
| chr2 | 205765349 | 205765368 | + | 19 | AC-316 | 2206 | 2836 | 3466 |
| chr2 | 206631959 | 206631973 | − | 14 | AC-317 | 2207 | 2837 | 3467 |
| chr2 | 206837280 | 206837306 | + | 26 | AC-318 | 2208 | 2838 | 3468 |
| chr2 | 213440109 | 213440124 | − | 15 | AC-319 | 2209 | 2839 | 3469 |
| chr2 | 216362283 | 216362296 | + | 13 | AC-320 | 2210 | 2840 | 3470 |
| chr2 | 217910713 | 217910762 | + | 49 | AC-321 | 2211 | 2841 | 3471 |
| chr2 | 217986730 | 217986762 | + | 32 | AC-322 | 2212 | 2842 | 3472 |
| chr2 | 218856787 | 218856822 | − | 35 | AC-323 | 2213 | 2843 | 3473 |
| chr2 | 219917944 | 219917978 | − | 34 | AC-324 | 2214 | 2844 | 3474 |
| chr2 | 220466852 | 220466875 | − | 23 | AC-325 | 2215 | 2845 | 3475 |
| chr2 | 226565658 | 226565678 | + | 20 | AC-326 | 2216 | 2846 | 3476 |
| chr2 | 227167895 | 227167908 | − | 13 | AC-327 | 2217 | 2847 | 3477 |
| chr2 | 229086910 | 229086938 | + | 28 | AC-328 | 2218 | 2848 | 3478 |
| chr2 | 232903436 | 232903461 | + | 25 | AC-329 | 2219 | 2849 | 3479 |
| chr2 | 234254256 | 234254275 | + | 19 | AC-330 | 2220 | 2850 | 3480 |
| chr2 | 236592543 | 236592555 | + | 12 | AC-331 | 2221 | 2851 | 3481 |
| chr2 | 237053674 | 237053687 | + | 13 | AC-332 | 2222 | 2852 | 3482 |
| chr2 | 238821668 | 238821682 | − | 14 | AC-333 | 2223 | 2853 | 3483 |
| chr20 | 1342147 | 1342172 | + | 25 | AC-334 | 2224 | 2854 | 3484 |
| chr20 | 11048993 | 11049022 | + | 29 | AC-335 | 2225 | 2855 | 3485 |
| chr20 | 16745165 | 16745199 | + | 34 | AC-336 | 2226 | 2856 | 3486 |
| chr20 | 19499916 | 19499932 | − | 16 | AC-337 | 2227 | 2857 | 3487 |
| chr20 | 20880235 | 20880284 | − | 49 | AC-338 | 2228 | 2858 | 3488 |
| chr20 | 22948907 | 22948922 | + | 15 | AC-339 | 2229 | 2859 | 3489 |
| chr20 | 23045210 | 23045227 | + | 17 | AC-340 | 2230 | 2860 | 3490 |
| chr20 | 36113408 | 36113455 | − | 47 | AC-341 | 2231 | 2861 | 3491 |
| chr20 | 38426995 | 38427033 | + | 38 | AC-342 | 2232 | 2862 | 3492 |
| chr20 | 41972646 | 41972658 | + | 12 | AC-343 | 2233 | 2863 | 3493 |
| chr20 | 43711429 | 43711441 | + | 12 | AC-344 | 2234 | 2864 | 3494 |
| chr20 | 46745042 | 46745060 | − | 18 | AC-345 | 2235 | 2865 | 3495 |
| chr20 | 47509448 | 47509461 | − | 13 | AC-346 | 2236 | 2866 | 3496 |
| chr20 | 50762751 | 50762781 | + | 30 | AC-347 | 2237 | 2867 | 3497 |
| chr20 | 52497640 | 52497672 | − | 32 | AC-348 | 2238 | 2868 | 3498 |
| chr20 | 52637926 | 52637955 | − | 29 | AC-349 | 2239 | 2869 | 3499 |
| chr20 | 52704971 | 52704984 | + | 13 | AC-350 | 2240 | 2870 | 3500 |
| chr21 | 17789253 | 17789287 | + | 34 | AC-351 | 2241 | 2871 | 3501 |
| chr21 | 20996105 | 20996147 | − | 42 | AC-352 | 2242 | 2872 | 3502 |
| chr21 | 34190142 | 34190162 | + | 20 | AC-353 | 2243 | 2873 | 3503 |
| chr21 | 35086392 | 35086425 | + | 33 | AC-354 | 2244 | 2874 | 3504 |
| chr21 | 36696792 | 36696808 | − | 16 | AC-355 | 2245 | 2875 | 3505 |
| chr21 | 41518473 | 41518504 | + | 31 | AC-356 | 2246 | 2876 | 3506 |
| chr21 | 42530595 | 42530655 | + | 60 | AC-357 | 2247 | 2877 | 3507 |
| chr22 | 31274832 | 31274844 | + | 12 | AC-358 | 2248 | 2878 | 3508 |
| chr22 | 32423577 | 32423607 | − | 30 | AC-359 | 2249 | 2879 | 3509 |
| chr22 | 33969491 | 33969503 | − | 12 | AC-360 | 2250 | 2880 | 3510 |
| chr22 | 35643499 | 35643525 | + | 26 | AC-361 | 2251 | 2881 | 3511 |
| chr22 | 36295401 | 36295422 | − | 21 | AC-362 | 2252 | 2882 | 3512 |
| chr3 | 14622530 | 14622562 | − | 32 | AC-363 | 2253 | 2883 | 3513 |
| chr3 | 15705328 | 15705340 | − | 12 | AC-364 | 2254 | 2884 | 3514 |
| chr3 | 18399927 | 18399939 | − | 12 | AC-365 | 2255 | 2885 | 3515 |
| chr3 | 22963762 | 22963785 | + | 23 | AC-366 | 2256 | 2886 | 3516 |
| chr3 | 35302794 | 35302838 | + | 44 | AC-367 | 2257 | 2887 | 3517 |
| chr3 | 37921607 | 37921644 | + | 37 | AC-368 | 2258 | 2888 | 3518 |
| chr3 | 48845469 | 48845481 | − | 12 | AC-369 | 2259 | 2889 | 3519 |
| chr3 | 49780309 | 49780342 | + | 33 | AC-370 | 2260 | 2890 | 3520 |
| chr3 | 53586276 | 53586320 | − | 44 | AC-371 | 2261 | 2891 | 3521 |
| chr3 | 61065268 | 61065295 | + | 27 | AC-372 | 2262 | 2892 | 3522 |
| chr3 | 61637728 | 61637741 | − | 13 | AC-373 | 2263 | 2893 | 3523 |
| chr3 | 66487585 | 66487600 | + | 15 | AC-374 | 2264 | 2894 | 3524 |
| chr3 | 83182081 | 83182109 | − | 28 | AC-375 | 2265 | 2895 | 3525 |
| chr3 | 86495672 | 86495686 | − | 14 | AC-376 | 2266 | 2896 | 3526 |
| chr3 | 97255144 | 97255181 | − | 37 | AC-377 | 2267 | 2897 | 3527 |
| chr3 | 99205670 | 99205706 | − | 36 | AC-378 | 2268 | 2898 | 3528 |
| chr3 | 109841830 | 109841859 | − | 29 | AC-379 | 2269 | 2899 | 3529 |
| chr3 | 115955645 | 115955657 | − | 12 | AC-380 | 2270 | 2900 | 3530 |
| chr3 | 117756399 | 117756411 | + | 12 | AC-381 | 2271 | 2901 | 3531 |
| chr3 | 123275925 | 123275938 | − | 13 | AC-382 | 2272 | 2902 | 3532 |
| chr3 | 126124110 | 126124148 | + | 38 | AC-383 | 2273 | 2903 | 3533 |
| chr3 | 128207566 | 128207604 | + | 38 | AC-384 | 2274 | 2904 | 3534 |
| chr3 | 134308065 | 134308077 | + | 12 | AC-385 | 2275 | 2905 | 3535 |
| chr3 | 137610467 | 137610490 | + | 23 | AC-386 | 2276 | 2906 | 3536 |
| chr3 | 142964237 | 142964267 | − | 30 | AC-387 | 2277 | 2907 | 3537 |
| chr3 | 150745240 | 150745278 | + | 38 | AC-388 | 2278 | 2908 | 3538 |
| chr3 | 157087700 | 157087720 | − | 20 | AC-389 | 2279 | 2909 | 3539 |
| chr3 | 175262575 | 175262609 | − | 34 | AC-390 | 2280 | 2910 | 3540 |
| chr3 | 175342505 | 175342533 | − | 28 | AC-391 | 2281 | 2911 | 3541 |
| chr3 | 179426975 | 179426987 | + | 12 | AC-392 | 2282 | 2912 | 3542 |
| chr3 | 186964274 | 186964286 | + | 12 | AC-393 | 2283 | 2913 | 3543 |
| chr3 | 188811351 | 188811385 | + | 34 | AC-394 | 2284 | 2914 | 3544 |
| chr3 | 188819027 | 188819071 | − | 44 | AC-395 | 2285 | 2915 | 3545 |
| chr3 | 189281542 | 189281554 | − | 12 | AC-396 | 2286 | 2916 | 3546 |
| chr4 | 3218933 | 3218947 | − | 14 | AC-397 | 2287 | 2917 | 3547 |
| chr4 | 10894438 | 10894474 | − | 36 | AC-398 | 2288 | 2918 | 3548 |
| chr4 | 13899844 | 13899895 | + | 51 | AC-399 | 2289 | 2919 | 3549 |
| chr4 | 25007578 | 25007626 | − | 48 | AC-400 | 2290 | 2920 | 3550 |
| chr4 | 25957671 | 25957730 | − | 59 | AC-401 | 2291 | 2921 | 3551 |
| chr4 | 31671125 | 31671145 | + | 20 | AC-402 | 2292 | 2922 | 3552 |
| chr4 | 33960900 | 33960938 | + | 38 | AC-403 | 2293 | 2923 | 3553 |
| chr4 | 36654887 | 36654930 | + | 43 | AC-404 | 2294 | 2924 | 3554 |
| chr4 | 38508529 | 38508567 | + | 38 | AC-405 | 2295 | 2925 | 3555 |
| chr4 | 44354216 | 44354229 | − | 13 | AC-406 | 2296 | 2926 | 3556 |
| chr4 | 45865788 | 45865800 | − | 12 | AC-407 | 2297 | 2927 | 3557 |
| chr4 | 65672409 | 65672447 | + | 38 | AC-408 | 2298 | 2928 | 3558 |
| chr4 | 65871252 | 65871272 | + | 20 | AC-409 | 2299 | 2929 | 3559 |
| chr4 | 76888192 | 76888240 | + | 48 | AC-410 | 2300 | 2930 | 3560 |
| chr4 | 81226274 | 81226297 | − | 23 | AC-411 | 2301 | 2931 | 3561 |
| chr4 | 81646328 | 81646375 | − | 47 | AC-412 | 2302 | 2932 | 3562 |
| chr4 | 83682203 | 83682245 | + | 42 | AC-413 | 2303 | 2933 | 3563 |
| chr4 | 84489018 | 84489064 | − | 46 | AC-414 | 2304 | 2934 | 3564 |
| chr4 | 90107729 | 90107760 | − | 31 | AC-415 | 2305 | 2935 | 3565 |
| chr4 | 102052609 | 102052623 | + | 14 | AC-416 | 2306 | 2936 | 3566 |
| chr4 | 104894587 | 104894612 | + | 25 | AC-417 | 2307 | 2937 | 3567 |
| chr4 | 108540472 | 108540514 | − | 42 | AC-418 | 2308 | 2938 | 3568 |
| chr4 | 109265518 | 109265568 | − | 50 | AC-419 | 2309 | 2939 | 3569 |
| chr4 | 116786381 | 116786427 | − | 46 | AC-420 | 2310 | 2940 | 3570 |
| chr4 | 128389652 | 128389698 | − | 46 | AC-421 | 2311 | 2941 | 3571 |
| chr4 | 137206883 | 137206895 | − | 12 | AC-422 | 2312 | 2942 | 3572 |
| chr4 | 145177942 | 145177955 | + | 13 | AC-423 | 2313 | 2943 | 3573 |
| chr4 | 145805125 | 145805161 | − | 36 | AC-424 | 2314 | 2944 | 3574 |
| chr4 | 145856682 | 145856724 | + | 42 | AC-425 | 2315 | 2945 | 3575 |
| chr4 | 148412821 | 148412842 | + | 21 | AC-426 | 2316 | 2946 | 3576 |
| chr4 | 151509759 | 151509788 | − | 29 | AC-427 | 2317 | 2947 | 3577 |
| chr4 | 154554344 | 154554377 | − | 33 | AC-428 | 2318 | 2948 | 3578 |
| chr4 | 157867966 | 157867985 | + | 19 | AC-429 | 2319 | 2949 | 3579 |
| chr4 | 158768315 | 158768354 | − | 39 | AC-430 | 2320 | 2950 | 3580 |
| chr4 | 171948962 | 171948987 | + | 25 | AC-431 | 2321 | 2951 | 3581 |
| chr4 | 173523993 | 173524024 | + | 31 | AC-432 | 2322 | 2952 | 3582 |
| chr4 | 181073679 | 181073716 | − | 37 | AC-433 | 2323 | 2953 | 3583 |
| chr5 | 914720 | 914741 | − | 21 | AC-434 | 2324 | 2954 | 3584 |
| chr5 | 2997205 | 2997225 | − | 20 | AC-435 | 2325 | 2955 | 3585 |
| chr5 | 5725644 | 5725674 | − | 30 | AC-436 | 2326 | 2956 | 3586 |
| chr5 | 8532870 | 8532882 | + | 12 | AC-437 | 2327 | 2957 | 3587 |
| chr5 | 9917710 | 9917726 | + | 16 | AC-438 | 2328 | 2958 | 3588 |
| chr5 | 16629570 | 16629617 | − | 47 | AC-439 | 2329 | 2959 | 3589 |
| chr5 | 22869460 | 22869491 | + | 31 | AC-440 | 2330 | 2960 | 3590 |
| chr5 | 36789014 | 36789039 | − | 25 | AC-441 | 2331 | 2961 | 3591 |
| chr5 | 38131972 | 38131989 | + | 17 | AC-442 | 2332 | 2962 | 3592 |
| chr5 | 53824869 | 53824898 | − | 29 | AC-443 | 2333 | 2963 | 3593 |
| chr5 | 59205653 | 59205699 | + | 46 | AC-444 | 2334 | 2964 | 3594 |
| chr5 | 73279446 | 73279458 | − | 12 | AC-445 | 2335 | 2965 | 3595 |
| chr5 | 74060374 | 74060387 | − | 13 | AC-446 | 2336 | 2966 | 3596 |
| chr5 | 77971951 | 77971963 | + | 12 | AC-447 | 2337 | 2967 | 3597 |
| chr5 | 78018671 | 78018701 | + | 30 | AC-448 | 2338 | 2968 | 3598 |
| chr5 | 89533569 | 89533581 | − | 12 | AC-449 | 2339 | 2969 | 3599 |
| chr5 | 100039934 | 100039949 | + | 15 | AC-450 | 2340 | 2970 | 3600 |
| chr5 | 102630348 | 102630361 | + | 13 | AC-451 | 2341 | 2971 | 3601 |
| chr5 | 105795040 | 105795087 | + | 47 | AC-452 | 2342 | 2972 | 3602 |
| chr5 | 106381874 | 106381887 | − | 13 | AC-453 | 2343 | 2973 | 3603 |
| chr5 | 108628093 | 108628109 | − | 16 | AC-454 | 2344 | 2974 | 3604 |
| chr5 | 110624008 | 110624020 | − | 12 | AC-455 | 2345 | 2975 | 3605 |
| chr5 | 116438053 | 116438073 | + | 20 | AC-456 | 2346 | 2976 | 3606 |
| chr5 | 116474278 | 116474310 | + | 32 | AC-457 | 2347 | 2977 | 3607 |
| chr5 | 135451181 | 135451193 | + | 12 | AC-458 | 2348 | 2978 | 3608 |
| chr5 | 137480303 | 137480344 | + | 41 | AC-459 | 2349 | 2979 | 3609 |
| chr5 | 139159689 | 139159701 | − | 12 | AC-460 | 2350 | 2980 | 3610 |
| chr5 | 144748341 | 144748387 | − | 46 | AC-461 | 2351 | 2981 | 3611 |
| chr5 | 146585259 | 146585302 | + | 43 | AC-462 | 2352 | 2982 | 3612 |
| chr5 | 150161415 | 150161428 | + | 13 | AC-463 | 2353 | 2983 | 3613 |
| chr5 | 154587485 | 154587506 | + | 21 | AC-464 | 2354 | 2984 | 3614 |
| chr5 | 157035448 | 157035463 | + | 15 | AC-465 | 2355 | 2985 | 3615 |
| chr5 | 158858373 | 158858386 | + | 13 | AC-466 | 2356 | 2986 | 3616 |
| chr5 | 161359440 | 161359467 | + | 27 | AC-467 | 2357 | 2987 | 3617 |
| chr5 | 165296411 | 165296424 | + | 13 | AC-468 | 2358 | 2988 | 3618 |
| chr5 | 173166004 | 173166037 | + | 33 | AC-469 | 2359 | 2989 | 3619 |
| chr5 | 174049445 | 174049463 | + | 18 | AC-470 | 2360 | 2990 | 3620 |
| chr5 | 176808837 | 176808873 | + | 36 | AC-471 | 2361 | 2991 | 3621 |
| chr6 | 11615873 | 11615896 | + | 23 | AC-472 | 2362 | 2992 | 3622 |
| chr6 | 18665733 | 18665746 | − | 13 | AC-473 | 2363 | 2993 | 3623 |
| chr6 | 21557228 | 21557240 | + | 12 | AC-474 | 2364 | 2994 | 3624 |
| chr6 | 22685540 | 22685552 | − | 12 | AC-475 | 2365 | 2995 | 3625 |
| chr6 | 25831656 | 25831668 | + | 12 | AC-476 | 2366 | 2996 | 3626 |
| chr6 | 26326576 | 26326591 | + | 15 | AC-477 | 2367 | 2997 | 3627 |
| chr6 | 35958717 | 35958730 | − | 13 | AC-478 | 2368 | 2998 | 3628 |
| chr6 | 40218844 | 40218856 | + | 12 | AC-479 | 2369 | 2999 | 3629 |
| chr6 | 43260942 | 43260958 | + | 16 | AC-480 | 2370 | 3000 | 3630 |
| chr6 | 43992143 | 43992155 | + | 12 | AC-481 | 2371 | 3001 | 3631 |
| chr6 | 45535992 | 45536004 | + | 12 | AC-482 | 2372 | 3002 | 3632 |
| chr6 | 46285124 | 46285136 | + | 12 | AC-483 | 2373 | 3003 | 3633 |
| chr6 | 50724763 | 50724781 | − | 18 | AC-484 | 2374 | 3004 | 3634 |
| chr6 | 52412748 | 52412760 | + | 12 | AC-485 | 2375 | 3005 | 3635 |
| chr6 | 67905996 | 67906016 | + | 20 | AC-486 | 2376 | 3006 | 3636 |
| chr6 | 71013985 | 71014013 | − | 28 | AC-487 | 2377 | 3007 | 3637 |
| chr6 | 82404255 | 82404267 | − | 12 | AC-488 | 2378 | 3008 | 3638 |
| chr6 | 84912911 | 84912931 | − | 20 | AC-489 | 2379 | 3009 | 3639 |
| chr6 | 90979513 | 90979551 | + | 38 | AC-490 | 2380 | 3010 | 3640 |
| chr6 | 93367616 | 93367631 | + | 15 | AC-491 | 2381 | 3011 | 3641 |
| chr6 | 100441435 | 100441449 | + | 14 | AC-492 | 2382 | 3012 | 3642 |
| chr6 | 101697340 | 101697382 | − | 42 | AC-493 | 2383 | 3013 | 3643 |
| chr6 | 108188502 | 108188552 | + | 50 | AC-494 | 2384 | 3014 | 3644 |
| chr6 | 109342833 | 109342859 | − | 26 | AC-495 | 2385 | 3015 | 3645 |
| chr6 | 112808037 | 112808055 | − | 18 | AC-496 | 2386 | 3016 | 3646 |
| chr6 | 117686852 | 117686899 | − | 47 | AC-497 | 2387 | 3017 | 3647 |
| chr6 | 124392523 | 124392536 | − | 13 | AC-498 | 2388 | 3018 | 3648 |
| chr6 | 124611705 | 124611718 | − | 13 | AC-499 | 2389 | 3019 | 3649 |
| chr6 | 131100960 | 131101006 | + | 46 | AC-500 | 2390 | 3020 | 3650 |
| chr6 | 142306681 | 142306694 | − | 13 | AC-501 | 2391 | 3021 | 3651 |
| chr6 | 150757561 | 150757580 | − | 19 | AC-502 | 2392 | 3022 | 3652 |
| chr6 | 155521023 | 155521057 | + | 34 | AC-503 | 2393 | 3023 | 3653 |
| chr6 | 156494300 | 156494337 | − | 37 | AC-504 | 2394 | 3024 | 3654 |
| chr6 | 162710374 | 162710415 | + | 41 | AC-505 | 2395 | 3025 | 3655 |
| chr6 | 164964828 | 164964849 | + | 21 | AC-506 | 2396 | 3026 | 3656 |
| chr7 | 2640392 | 2640404 | − | 12 | AC-507 | 2397 | 3027 | 3657 |
| chr7 | 6659561 | 6659575 | + | 14 | AC-508 | 2398 | 3028 | 3658 |
| chr7 | 10278035 | 10278047 | + | 12 | AC-509 | 2399 | 3029 | 3659 |
| chr7 | 11669971 | 11669992 | − | 21 | AC-510 | 2400 | 3030 | 3660 |
| chr7 | 15816101 | 15816116 | − | 15 | AC-511 | 2401 | 3031 | 3661 |
| chr7 | 18499080 | 18499103 | − | 23 | AC-512 | 2402 | 3032 | 3662 |
| chr7 | 27436646 | 27436658 | + | 12 | AC-513 | 2403 | 3033 | 3663 |
| chr7 | 31321320 | 31321356 | − | 36 | AC-514 | 2404 | 3034 | 3664 |
| chr7 | 33313036 | 33313065 | − | 29 | AC-515 | 2405 | 3035 | 3665 |
| chr7 | 35730091 | 35730107 | + | 16 | AC-516 | 2406 | 3036 | 3666 |
| chr7 | 36126622 | 36126646 | − | 24 | AC-517 | 2407 | 3037 | 3667 |
| chr7 | 41046876 | 41046888 | − | 12 | AC-518 | 2408 | 3038 | 3668 |
| chr7 | 49423670 | 49423683 | − | 13 | AC-519 | 2409 | 3039 | 3669 |
| chr7 | 53147640 | 53147653 | + | 13 | AC-520 | 2410 | 3040 | 3670 |
| chr7 | 53336267 | 53336300 | + | 33 | AC-521 | 2411 | 3041 | 3671 |
| chr7 | 54281367 | 54281380 | + | 13 | AC-522 | 2412 | 3042 | 3672 |
| chr7 | 66087243 | 66087283 | − | 40 | AC-523 | 2413 | 3043 | 3673 |
| chr7 | 68187087 | 68187120 | + | 33 | AC-524 | 2414 | 3044 | 3674 |
| chr7 | 69931112 | 69931148 | + | 36 | AC-525 | 2415 | 3045 | 3675 |
| chr7 | 70640572 | 70640594 | − | 22 | AC-526 | 2416 | 3046 | 3676 |
| chr7 | 74404520 | 74404532 | + | 12 | AC-527 | 2417 | 3047 | 3677 |
| chr7 | 74404793 | 74404805 | + | 12 | AC-528 | 2418 | 3048 | 3678 |
| chr7 | 78124681 | 78124698 | + | 17 | AC-529 | 2419 | 3049 | 3679 |
| chr7 | 79113811 | 79113837 | + | 26 | AC-530 | 2420 | 3050 | 3680 |
| chr7 | 89607949 | 89607976 | − | 27 | AC-531 | 2421 | 3051 | 3681 |
| chr7 | 104249356 | 104249387 | − | 31 | AC-532 | 2422 | 3052 | 3682 |
| chr7 | 111008579 | 111008595 | + | 16 | AC-533 | 2423 | 3053 | 3683 |
| chr7 | 115060128 | 115060142 | + | 14 | AC-534 | 2424 | 3054 | 3684 |
| chr7 | 117862056 | 117862075 | + | 19 | AC-535 | 2425 | 3055 | 3685 |
| chr7 | 119596454 | 119596491 | − | 37 | AC-536 | 2426 | 3056 | 3686 |
| chr7 | 121388235 | 121388261 | + | 26 | AC-537 | 2427 | 3057 | 3687 |
| chr7 | 124788365 | 124788396 | − | 31 | AC-538 | 2428 | 3058 | 3688 |
| chr7 | 125351681 | 125351726 | − | 45 | AC-539 | 2429 | 3059 | 3689 |
| chr7 | 135950348 | 135950363 | − | 15 | AC-540 | 2430 | 3060 | 3690 |
| chr7 | 137046728 | 137046764 | + | 36 | AC-541 | 2431 | 3061 | 3691 |
| chr7 | 137545482 | 137545496 | + | 14 | AC-542 | 2432 | 3062 | 3692 |
| chr7 | 137843756 | 137843796 | + | 40 | AC-543 | 2433 | 3063 | 3693 |
| chr7 | 145093880 | 145093901 | − | 21 | AC-544 | 2434 | 3064 | 3694 |
| chr7 | 151812948 | 151812961 | + | 13 | AC-545 | 2435 | 3065 | 3695 |
| chr7 | 152193684 | 152193700 | + | 16 | AC-546 | 2436 | 3066 | 3696 |
| chr7 | 157486364 | 157486382 | − | 18 | AC-547 | 2437 | 3067 | 3697 |
| chr8 | 2877350 | 2877369 | − | 19 | AC-548 | 2438 | 3068 | 3698 |
| chr8 | 5916479 | 5916522 | + | 43 | AC-549 | 2439 | 3069 | 3699 |
| chr8 | 16833227 | 16833239 | + | 12 | AC-550 | 2440 | 3070 | 3700 |
| chr8 | 17096955 | 17096997 | − | 42 | AC-551 | 2441 | 3071 | 3701 |
| chr8 | 18673202 | 18673219 | + | 17 | AC-552 | 2442 | 3072 | 3702 |
| chr8 | 22575902 | 22575933 | − | 31 | AC-553 | 2443 | 3073 | 3703 |
| chr8 | 29900159 | 29900193 | − | 34 | AC-554 | 2444 | 3074 | 3704 |
| chr8 | 37808558 | 37808588 | − | 30 | AC-555 | 2445 | 3075 | 3705 |
| chr8 | 56677789 | 56677815 | − | 26 | AC-556 | 2446 | 3076 | 3706 |
| chr8 | 60504361 | 60504373 | + | 12 | AC-557 | 2447 | 3077 | 3707 |
| chr8 | 64445861 | 64445874 | − | 13 | AC-558 | 2448 | 3078 | 3708 |
| chr8 | 69834086 | 69834129 | + | 43 | AC-559 | 2449 | 3079 | 3709 |
| chr8 | 69834735 | 69834780 | + | 45 | AC-560 | 2450 | 3080 | 3710 |
| chr8 | 72983955 | 72983989 | + | 34 | AC-561 | 2451 | 3081 | 3711 |
| chr8 | 74296019 | 74296045 | + | 26 | AC-562 | 2452 | 3082 | 3712 |
| chr8 | 99657470 | 99657495 | − | 25 | AC-563 | 2453 | 3083 | 3713 |
| chr8 | 100292435 | 100292469 | − | 34 | AC-564 | 2454 | 3084 | 3714 |
| chr8 | 101967840 | 101967867 | + | 27 | AC-565 | 2455 | 3085 | 3715 |
| chr8 | 102463303 | 102463317 | − | 14 | AC-566 | 2456 | 3086 | 3716 |
| chr8 | 108700130 | 108700168 | − | 38 | AC-567 | 2457 | 3087 | 3717 |
| chr8 | 108923709 | 108923753 | − | 44 | AC-568 | 2458 | 3088 | 3718 |
| chr8 | 111782619 | 111782639 | + | 20 | AC-569 | 2459 | 3089 | 3719 |
| chr8 | 113897306 | 113897345 | + | 39 | AC-570 | 2460 | 3090 | 3720 |
| chr8 | 114293551 | 114293563 | − | 12 | AC-571 | 2461 | 3091 | 3721 |
| chr8 | 115306130 | 115306143 | + | 13 | AC-572 | 2462 | 3092 | 3722 |
| chr8 | 115546581 | 115546612 | + | 31 | AC-573 | 2463 | 3093 | 3723 |
| chr9 | 3172768 | 3172798 | + | 30 | AC-574 | 2464 | 3094 | 3724 |
| chr9 | 7622615 | 7622629 | − | 14 | AC-575 | 2465 | 3095 | 3725 |
| chr9 | 7843389 | 7843402 | + | 13 | AC-576 | 2466 | 3096 | 3726 |
| chr9 | 16276520 | 16276534 | + | 14 | AC-577 | 2467 | 3097 | 3727 |
| chr9 | 24072795 | 24072840 | − | 45 | AC-578 | 2468 | 3098 | 3728 |
| chr9 | 31036268 | 31036292 | + | 24 | AC-579 | 2469 | 3099 | 3729 |
| chr9 | 32967307 | 32967357 | + | 50 | AC-580 | 2470 | 3100 | 3730 |
| chr9 | 36644209 | 36644252 | − | 43 | AC-581 | 2471 | 3101 | 3731 |
| chr9 | 36814754 | 36814766 | − | 12 | AC-582 | 2472 | 3102 | 3732 |
| chr9 | 68529164 | 68529211 | − | 47 | AC-583 | 2473 | 3103 | 3733 |
| chr9 | 70118297 | 70118348 | + | 51 | AC-584 | 2474 | 3104 | 3734 |
| chr9 | 70120254 | 70120267 | + | 13 | AC-585 | 2475 | 3105 | 3735 |
| chr9 | 75599977 | 75600015 | + | 38 | AC-586 | 2476 | 3106 | 3736 |
| chr9 | 79333729 | 79333742 | − | 13 | AC-587 | 2477 | 3107 | 3737 |
| chr9 | 89483736 | 89483748 | − | 12 | AC-588 | 2478 | 3108 | 3738 |
| chr9 | 91239745 | 91239757 | + | 12 | AC-589 | 2479 | 3109 | 3739 |
| chr9 | 94821970 | 94822012 | + | 42 | AC-590 | 2480 | 3110 | 3740 |
| chr9 | 102201485 | 102201510 | − | 25 | AC-591 | 2481 | 3111 | 3741 |
| chr9 | 105161311 | 105161342 | + | 31 | AC-592 | 2482 | 3112 | 3742 |
| chr9 | 107603638 | 107603661 | − | 23 | AC-593 | 2483 | 3113 | 3743 |
| chr9 | 114010999 | 114011011 | + | 12 | AC-594 | 2484 | 3114 | 3744 |
| chr9 | 121479711 | 121479723 | + | 12 | AC-595 | 2485 | 3115 | 3745 |
| chr9 | 122676111 | 122676139 | − | 28 | AC-596 | 2486 | 3116 | 3746 |
| chr9 | 125815693 | 125815718 | − | 25 | AC-597 | 2487 | 3117 | 3747 |
| chr9 | 127086123 | 127086135 | + | 12 | AC-598 | 2488 | 3118 | 3748 |
| chr9 | 130789525 | 130789547 | + | 22 | AC-599 | 2489 | 3119 | 3749 |
| chr9 | 132840406 | 132840448 | + | 42 | AC-600 | 2490 | 3120 | 3750 |
| chr9 | 135077150 | 135077168 | + | 18 | AC-601 | 2491 | 3121 | 3751 |
| chrX | 3603350 | 3603362 | + | 12 | AC-602 | 2492 | 3122 | 3752 |
| chrX | 11068536 | 11068574 | − | 38 | AC-603 | 2493 | 3123 | 3753 |
| chrX | 11630010 | 11630030 | − | 20 | AC-604 | 2494 | 3124 | 3754 |
| chrX | 11663768 | 11663782 | + | 14 | AC-605 | 2495 | 3125 | 3755 |
| chrX | 16007548 | 16007583 | − | 35 | AC-606 | 2496 | 3126 | 3756 |
| chrX | 16776434 | 16776474 | − | 40 | AC-607 | 2497 | 3127 | 3757 |
| chrX | 16864153 | 16864168 | + | 15 | AC-608 | 2498 | 3128 | 3758 |
| chrX | 29069628 | 29069670 | + | 42 | AC-609 | 2499 | 3129 | 3759 |
| chrX | 30746990 | 30747008 | − | 18 | AC-610 | 2500 | 3130 | 3760 |
| chrX | 40167735 | 40167747 | + | 12 | AC-611 | 2501 | 3131 | 3761 |
| chrX | 42183785 | 42183833 | − | 48 | AC-612 | 2502 | 3132 | 3762 |
| chrX | 45696781 | 45696803 | − | 22 | AC-613 | 2503 | 3133 | 3763 |
| chrX | 48471856 | 48471869 | + | 13 | AC-614 | 2504 | 3134 | 3764 |
| chrX | 72429741 | 72429754 | − | 13 | AC-615 | 2505 | 3135 | 3765 |
| chrX | 74457676 | 74457689 | + | 13 | AC-616 | 2506 | 3136 | 3766 |
| chrX | 78121402 | 78121425 | − | 23 | AC-617 | 2507 | 3137 | 3767 |
| chrX | 82818338 | 82818351 | − | 13 | AC-618 | 2508 | 3138 | 3768 |
| chrX | 100561901 | 100561918 | + | 17 | AC-619 | 2509 | 3139 | 3769 |
| chrX | 106955518 | 106955541 | + | 23 | AC-620 | 2510 | 3140 | 3770 |
| chrX | 110947528 | 110947542 | + | 14 | AC-621 | 2511 | 3141 | 3771 |
| chrX | 131203326 | 131203338 | − | 12 | AC-622 | 2512 | 3142 | 3772 |
| chrX | 136311394 | 136311430 | + | 36 | AC-623 | 2513 | 3143 | 3773 |
| chrX | 137626789 | 137626814 | − | 25 | AC-624 | 2514 | 3144 | 3774 |
| chrX | 141714377 | 141714400 | + | 23 | AC-625 | 2515 | 3145 | 3775 |
| chrX | 142998165 | 142998193 | − | 28 | AC-626 | 2516 | 3146 | 3776 |
| chrX | 146107858 | 146107870 | − | 12 | AC-627 | 2517 | 3147 | 3777 |
| chrX | 147756133 | 147756154 | + | 21 | AC-628 | 2518 | 3148 | 3778 |
| chrX | 151615298 | 151615311 | + | 13 | AC-629 | 2519 | 3149 | 3779 |
| chrX | 154675789 | 154675801 | + | 12 | AC-630 | 2520 | 3150 | 3780 |

### Example 3B: Construction of Library for Enrichment Panel

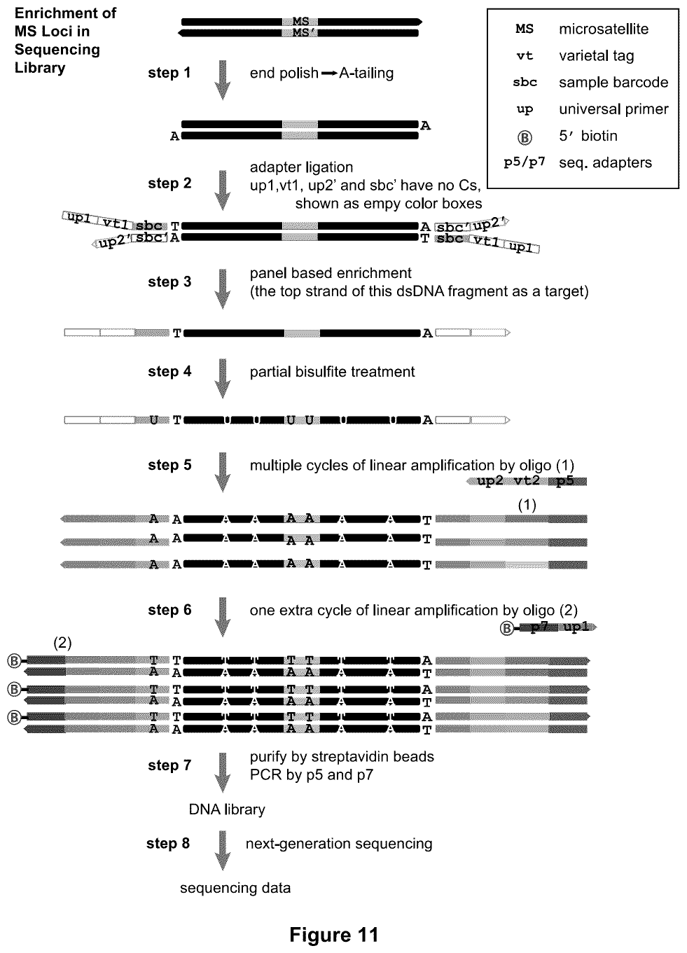

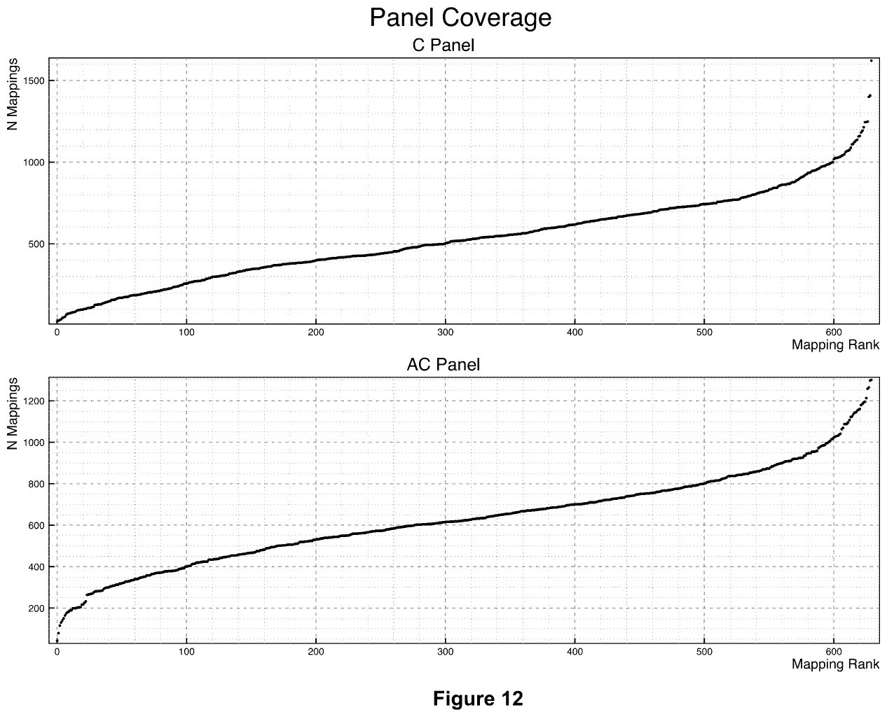

As shown in FIG. 11, DNA fragmentase is used to fragment one (1) microgram of genomic DNA to a target size of 300 bp. After end-polishing and A-tailing, the double-stranded DNA fragments is adapted with mutation-resistant fork-tailed primers augmented with a varietal tag (see Example 5). The adapted DNA fragments are then denatured and captured with either: (i) the C-MS panel of 630 loci or (ii) the AC-MS panel of 630 loci. For each panel, the DNA strand in which the microsatellite is C or AC is targeted. Sometimes this is the reference strand and sometimes the reverse complement of the reference strand.

The panels are comprised of 60 bp oligonucleotides complementary to the targeted flank sequences with a biotin covalently attached at the 5′ end. After hybridization capture of the target DNA, capture oligos are pulled down with streptavidin along with the hybridized target DNA. After release from the capture reagents, the templates are subjected to partial bisulfite conversion, as described above. Then the mutated target molecules are linearly amplified from the mutation resistant handle for 20 cycles, adding a second unique varietal tag to each linear copy and one of the two sequencing primers (read 1 primer, P5). Double-stranded DNA fragments are then generated by another linear amplification including the second sequencing primer (read 2 primer, P7). This is followed by size selection for the length range from 300 bp to 500 bp. This is then followed by 12 rounds of exponential amplification to make the final sequencing libraries.

### Example 3C: Analysis of Sequencing Reads and Method Performance

After sequencing, the resultant reads were analyzed for proper structure. From reads with the proper structure, the varietal tags and universal primer regions are extracted and then the genomic segments are mapped to the genome. Mapping was performed by first fully converting the genomic segments from each read-pair: read 1 is converted G to A and read 2 is converted C to T. This is the expected pattern of conversion from the incorporation of the sequencing primers since the linear amplification that adds the P5 primer primes off of the C->U converted template. Mapping the opposite conversion was tested and 97% of reads have a higher mapping score with read 1 converted G->A, as expected.

The inserts are then mapped to two possible genomes, one where every C is converted to T (ref_CT) and one where every G is converted to an A (ref_GA), corresponding to whether the mutated template derived from the reference strand (ref_CT) or from the reverse complement (ref_GA). The mapping with the best quality score is selected.

The proportion of mapped reads that map to the target regions is then examined. While the target accounts for only 0.002% of the genome, after enrichment 43% and 33% of mapped reads fall within the target region for C-MS and AC-MS respectively. It was also noted that coverage over target loci appears relatively uniform. (See FIG. 11)

As described above, the capture oligos are stranded, designed to be complementary to strand containing the C or AC repeat. The C and AC targets are divided by those where the C-containing repeat is on the reference strand (ref) and those where the C-containing repeat is on the complement of the reference strand (ref_complement). It was found that 99.97% of the ref reads map to the ref_CT genome and 99.98% of the ref_complement reads map the ref_A genome.

### Example 3D: Other Variations on Enrichment Library Protocol

In another realization of this process, mutagenesis and/or amplification is performed before capture, using redundancy (or not) to reduce error.

In another realization of this process, the first-round copies are partially mutagenized rather than the initial templates.

In another realization of this process, barcoded samples are pooled prior to enrichment.

In another realization of this process, linear amplification with a mixture of dNTP containing methyl-dCTP is performed before capture, followed by complete conversion of unmethylated cytosines, such that the first copies are mutagenized. Template redundancy may or may not be used to reduce error.

In another realization of this process, the library is enriched twice to improve on-target rates.

### Example 4—Whole Genome Library from Mutagenized Templates

To determine microsatellite lengths in all C containing microsatellites across the genome, a whole genome sequencing (WGS) library is generated that has undergone bisulfite mutagenesis, with varietal tags added to the initial templates and independently to the first copies. This protocol is highly similar to that shown in FIG. 11, with step 3 (panel enrichment) skipped.

The protocol for this example has the following steps:
(a) fragmentation of genomic DNA to a target size of 300 bp;
(b) end-polishing and A-tailing;
(c) adapting the double-stranded DNA fragments with mutation-resistant fork-tailed primers augmented with a varietal tag;
(d) partial mutagenesis with bisulfite treatment;
(e) linear amplification for 20 cycles, adding a second unique varietal tag to each linear copy and one of the two sequencing primers (read 1 primer, P5);
(f) one round of linear amplification to make double-stranded DNA fragments including the second sequencing primer (read 2 primer, P7);
(g) size selection for the length range from 300 bp to 500 bp;
(h) exponential amplification for 12 rounds to make the final sequencing libraries.

There are many variations that can be incorporated into this example. In particular, variation in the number of rounds of linear and exponential amplification; the precise design of the fish tail primers; and so on.

### Example 5—Applications of Measuring Microsatellite Length

### Example 5A: Determining Cancer Signature

In this example, a cancer microsatellite length signature is when a cancer has microsatellite length variation from the known germline of the host. To determine the cancer signature, the protocols described herein are applied to DNA extracted from a biopsy of a cancer, whether primary or metastatic, and to DNA extracted from the blood cells, or some other patient-derived sample, that represents the germline of that patient. The signature of the cancer is then the statistically robust differences between the cancer and normal sample over all the loci that have been examined. This can be done over the entire genome, or by using panels to enrich certain loci.

It is desirable to know the signature from the cancer for two main reasons. First, once known, the signature will help one follow the cancer load at presentation and over time (see Example 5D). It could help identify a new lesion as metastatic, and whether the cancer has spread to the lymph nodes or surgical margins. The signature may also provide a measure of proportion of DNA in a biopsy that is cancer, that is, the cancer cellularity. If the cancer is sampled from more than one location, the signature at each location will provide evidence of genomic heterogeneity and spread, and for residual disease at the surgical margins.

Second, the degree of MSLV will help determine if the patient has microsatellite instability (MSI). MSI is a useful determination, as it predicts outcome and guides therapeutic options. Unlike other methods of evaluating MSI, the methods described herein should provide a quantitative degree of instability, which may have clinical utility.

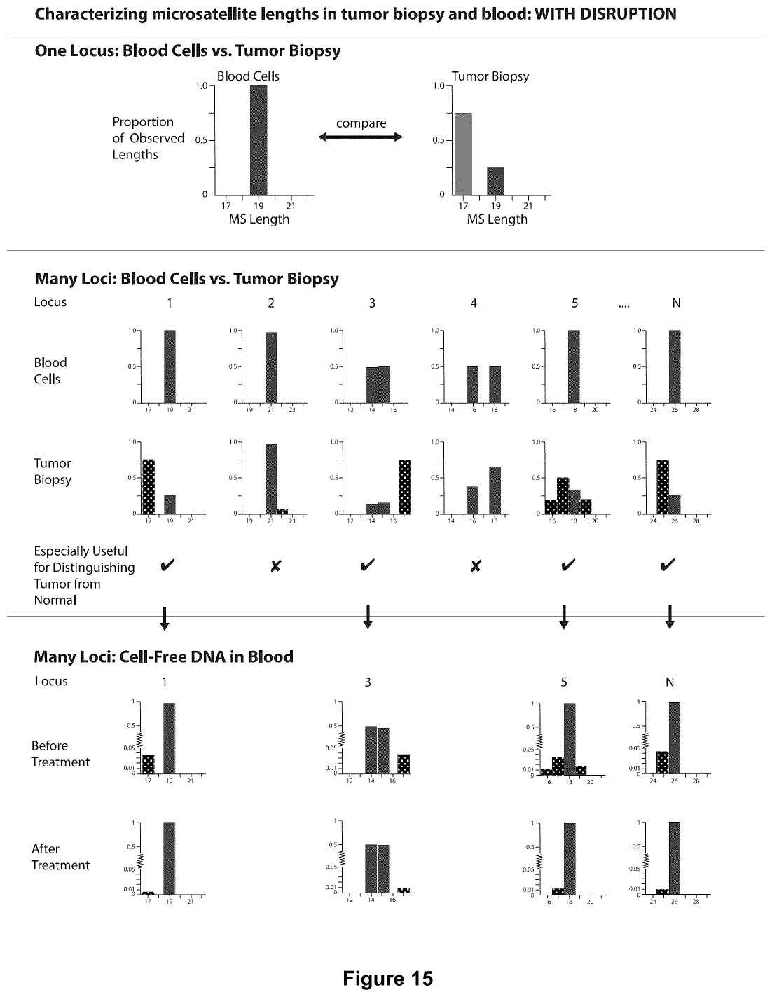

See Example 7 and FIG. 15 for further illustration on this topic.

### Example 5B: Early Detection of Cancer by Analysis Cell Free DNA in Blood

It is of value to detect a neoplasm at the earliest possible time, so action taken before a life threatening malignancy emerges. The need is especially critical in persons with a prior history of cancer, a family history of cancer, persons exposed to carcinogens, for example smokers, and in the elderly. Cancers release DNA and cells into the blood stream, and MSLV can be used to detect these at the earliest possible times. A test can be based on circulating tumor cells (ct-cells) or cell-free DNA (cf-DNA) (Georgiadis, A. et al., 2019; Abbosh, C. et al., 2017; Coombes, R. C. et al., 2019; Cristiano, S. et al., 2019; Garcia-Murillas, I. et al., 2015; Phallen, J. et al., 2017; Tie, J. et al., 2016). In this example the latter is considered because most studies, including the inventors' own, report higher signal in cf-DNA than in ct-cells. Other patient specimens could be tested, such as urine, fecal matter or sputum.

Because a prior signature is not known, it is desirable to survey many microsatellites. The analysis is performed on a panel of microsatellite loci, N, as described in FIG. 11. The presence of C in the microsatellite enables one to perform partial bisulfite mutagenesis which targets that nucleotide. N=10\hat{ }3 would likely suffice, but it will be determined empirically over time what values of N are the most cost effective, which loci might be the most valuable, and how many variant loci are needed for confident detection of neoplastic growth.

Because microsatellite lengths are so variable, any significant new growth in the body is likely to have some loci with a new MS length, differing from the germline-inherited lengths. Cell death would result in the release of such DNA into the circulation, although in small amounts. Provided a detection method is used with an error rate below the concentration of these substances in the cf-DNA, they could be detected. The methods described herein based on partial bisulfite mutagenesis of a template, further requiring at least two linear copies of that template that agree, would have an error estimated to be on the order of one part per ten million, even lower with three linear copies, and far below the needed error rate for MSLV detection of rare molecules in cf-DNA.

Any result would need to be considered in the context of normal variation. Some amount of variation beyond the germline might be common in healthy persons. For example, somatic clones probably arise during development that would be characterized by somatic variation at some loci characteristic for an adult tissue. As regeneration happens throughout life, this tissue specific signature for healthy persons may vary. When those cells die, their DNA would also appear in cf-DNA. So, for example, someone with active hepatitis, might be releasing DNA from hepatic cells, and this might be detectable by the methods described herein. Thus, each person will have a baseline of low frequency length variants, but one which might vary depending on their condition and age.

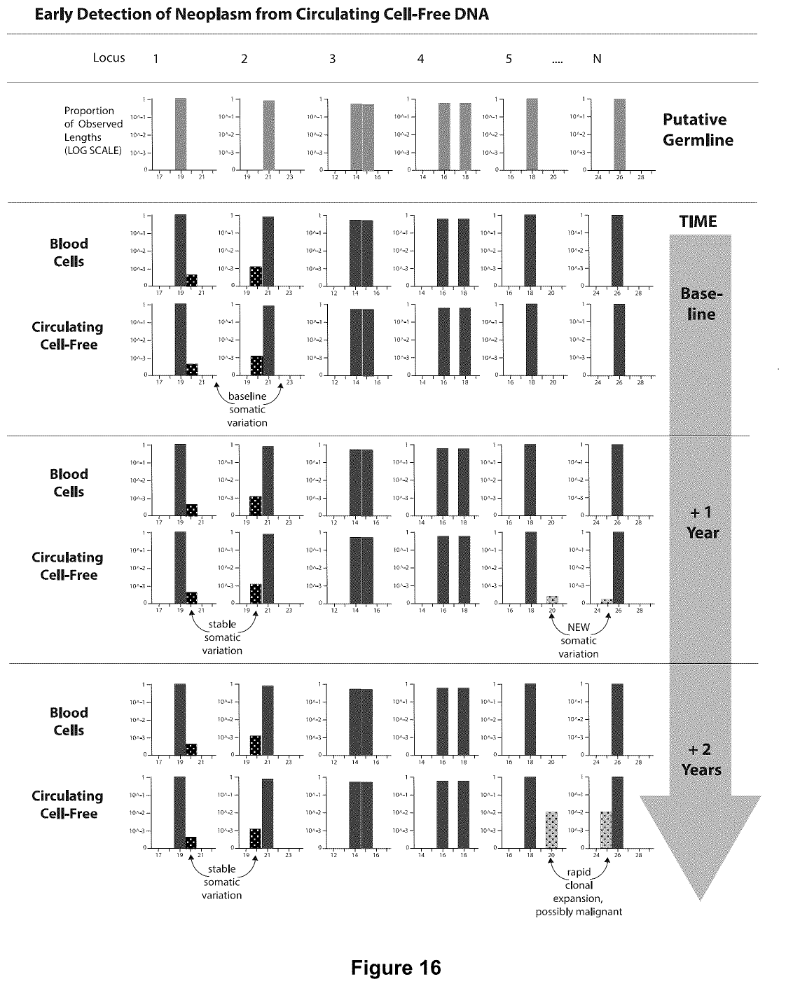

Ideally, a person at risk should be monitored over time. Any detection of a set of loci with new lengths would be re-examined on a repeat assay. An increase in the frequency of any of them would be an alarming signal, evidence of a clone which is expanding. This would signal the need for further actions, such as imaging or additional blood tests. Also, one would like to distinguish signals that emanate from an expanding new growth from the signal that might come from the blood cells themselves, which are probably in a constant state of expansion and replacement, for example secondary to inflammation and infections. Thus, in addition to the dynamic monitoring of MSLV, one would look for MSLV in the blood cells over the same panel of loci. See Example 8 and FIG. 16 for further illustration on this topic.

### Example 5C: Detecting Cancer in Exploratory Biopsies

A common clinical problem is to determine from a biopsy whether a person has a malignant growth and requires further treatment. This occurs for examples when examining a skin growth, biopsies of polyps during colonoscopy, or while searching for prostate cancer in core biopsies. In these cases, signature is not initially known, but one can reasonably expect the proportion of malignant cells would be high, as in the first application example. In addition to finding the proportion of a sample that is new growth, the signature of the new growth is thereby determined; the degree of MSI (microsatellite instability) is determined; and by multiple site biopsies and examination of signature in blood one can also measure tumor heterogeneity and spread.

### Example 5D: Measuring Cancer Load by Analysis of Cells and Cell Free DNA in Blood

Solid cancers leave traces in the blood (in the form of cell-free DNA and cells) and other samples such as cerebral-spinal fluid, urine, sputum and fecal matter that can be monitored without signature. Assays without signature require that large numbers of loci are assayed, because of the need to detect new emergent resistant cancer clones.

### Example 5E: Monitoring Residual Cancer

One clear application of the method is to monitoring residual disease in the case of leukemia. In this instance sample depth should be very high, on the order of 10\hat{ }6, to be able to detect very low levels of residual disease. Thus, for this application, signature would make the assays affordable, as the number of loci could then be reduced to 10 or 20 known loci variants in the presenting leukemia.

Because new leukemic clones can become chemo-resistant, and such cells might have new MSLV not detected at presentation, the broader screen, higher with higher number of loci and lower depth, such as described in application Example 1, might also be warranted.

### Example 5F: Monitoring Inflammatory Responses

The inflammatory response is characterized by the recruitment of cells of various lineages from the lymph nodes and bone marrow into the blood and to the site of inflammation. Often the cells are recruited from pre-existing somatic clones, and further clonal proliferation occurs. During this process the clonal expansions may generate new somatic variants of MSL, which can then be used to characterize that inflammatory response, and to detect its recurrence. For example, someone infected by a specific pathogen or having an autoimmune flare-up might mount an immune response characterized by the appearance in the blood of a specific MSLV spectra. The reappearance of that spectra again, perhaps years later, might indicate a similar inflammatory process has been triggered.

### Example 5G: Assessment of Male Sterility

Sometimes the germ line in males becomes dominated by a mutant clone, or a few mutant clones, especially as males age. This reduces fertility and can be associated with the emergence of de novo genetic disorders. This would be discoverable by the application of the method described herein to sperm DNA. In particular the abundance of a variant MS or a set of abundant MS not present in the patient's blood, could signal this as a cause. This would have application in the testing of sperm donors.

### Example 5H: Other Uses

Other possible applications are briefly listed here. Microsatellite length variation accurately and quantitatively measured, can be used to decipher the composition of complex populations, which is useful in agricultural and animal breeding. For the same reason, there are forensic applications in human populations, where individuals in large populations must be identified or traced. Other possible uses are testing pharmacologic and cosmetic compounds for their mutagenic properties.

### Example 6—Distinguishing Mixtures of Populations with Different Microsatellite Lengths

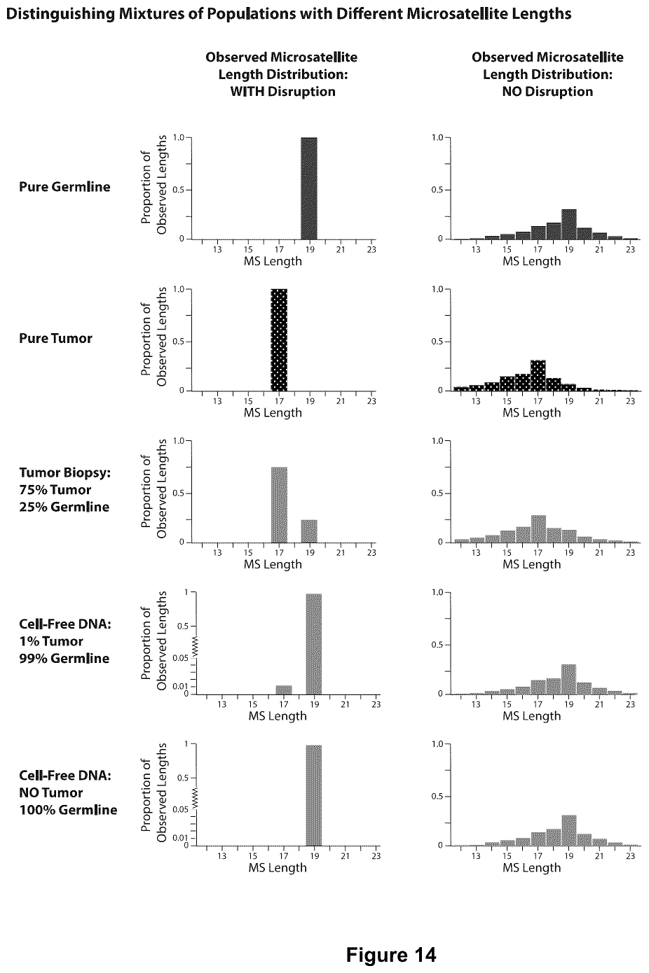

In this example (FIG. 14) the ability to distinguish DNA from mixed population of cells is illustrated, with and without microsatellite disruption, at various ratios of mixture. For simplicity, a locus is illustrated where one population of cells is homozygous for length 19 (the germ line) and the other is homozygous for length 17 (the tumor), as shown in the top two panels in dark grey and black with white dots, respectively. The mixture at 75%, 1% and 0% tumor are shown in grey, below. The distribution of lengths with disruption is shown on the left and the length distributions without disruption is seen on the right. It is evident in this simple case that by comparing the top right panel and the middle right panel one can infer the presence of length 17 in the tumor. One would not be able to say much more. There could be minor variants in the tumor that would be missed in analysis of reads without disruption; yet seen when the lengths are determined from the reads with disrupted microsatellites. The difference between the left and right become clear when looking at disproportionate mixtures. Without disruption, it is effectively impossible to distinguish a population of 1% tumor cells from a population without tumor (lower right two panels). This is straightforward task when distributions are derived from reads with disruption (lower left two panels).

### Example 7—Determining and Detecting the Tumor Microsatellite Signature in a Tumor Biopsy and Cell-Free DNA

FIG. 15 illustrates how one would profile the new variants in a tumor, and then monitor the tumor load in the patients before and after therapy.

In the top panel, analysis for one locus is illustrated, comparing DNA from blood cells to the DNA from a biopsy of a tumor. The observed length in blood is mainly or entirely 19 bp, while in the biopsy the length 19 is observed, but a new length of 17 is also observed, which in this case is the major component. The length of 19 is expected of the normal host stroma present in every cancer biopsy, while the ne length of 17 is inferred to be from the patient's cancer. From its abundance one can also infer that many, perhaps all, of the cells of the cancer have lost any of the normal allele length at this locus.

In the middle set of panels, a similar scenario at N loci for that patient is displayed. Locus 1 is the case just examined. At locus 2 a new but minor variant is observed, perhaps originating from the tumor, but certainly not present in the majority of cancer cells. At locus three, the patient's germline is heterozygous, and the cancer has developed a new microsatellite length. At the fourth locus, the patient is homozygous, but no new variant is seen in the cancer. At the fifth locus, more than one new variant is seen in the cancer, perhaps reflecting that the cancer has clonal substructure. The N-th locus is similar to the first. All of these loci may be useful for searching the cell free component of the blood, but first, third, fifth and N-th are especially useful, as they show some of the signature microsatellite length variation of the cancer. In the bottom panels, only those loci in the analysis of the cell free DNA are shown.

In the bottom panels the analysis of the blood before and after therapy is shown. At all the indicated loci, signature of the cancer is observed. The cancer is still present in the patient, although its load has been diminished by the therapy. Notably, not all variants from the fifth locus are present after treatment, suggesting that cells from this lineage of the cancer have been more successfully removed.

### Example 8—Early Detection of a Neoplasm from Circulating Cell-Free DNA

FIG. 16 illustrates how a neoplasm in the body might be detected from a time series of measurements of the cell free component of blood: baseline measurement at time zero, and further measurements in each of the two following years. Signal is detectable on the assumption that (1) cells from a neoplasm die and release their DNA into the circulation, where they join the DNA released by death of other normal stromal cells; (2) the neoplasm is largely clonal, or has major clonal components; (3) early in the process of clonal growth the neoplasm acquires new variation at a set of microsatellite loci; and (4) the population continues to expands over time.

On the top panel a putative profile of microsatellite lengths at N loci (gray) is shown. Some loci are homozygous (1, 2, 5 and N) and some are heterozygous (3 and 4). At time zero, the baseline, the main pattern is observed in the DNA of blood cells (dark grey) and also in the cell free component of blood (dark grey). In addition, there may well be somatic variation that has arisen at some loci at the time the patient's cell free circulating DNA is first assayed (the pair of arrows on the left, loci 1 and 2, in black with white dots). The source of these variants may well be clonal subpopulations of the normal components of the blood. The proportion of these microsatellite length variants may wax and wane during the patient's lifetime, but are relatively stable, and in any are seen to reflect variants in the blood cell population.

In year one, new somatic variation appears at loci 5 and N (right arrows, grey with black dots), and this variation is not seen in the blood cells. By the second year, these new variants have undergone a rapid clonal expansion, and the possibility of a malignancy should be considered as the source.

### Example 9—Estimating Error in Length Determination Using Independent Paired Reads

It is straightforward to calculate error rates in microsatellite length measurement when the ground truth (i.e. actual microsatellite length) is known, such as in the model experiments based on synthetic templates. When that truth is not known, it is still possible to calculate error rates provided that one has two independent reads that match for the same template molecule or the same first copy of a template molecule. Under some simplifying assumptions (for example, a read has not mutated twice, that all errors lead to the same length) one can calculate error from the proportion of all matched pairs that do not agree on the length. The proportion will be 2×(1−p)×p where p is the error rate. This method is called the method of matched pair read agreement. In Table 11A and Table 11B calculations were carried out on the data from synthetic templates, and the values almost exactly match those expected from read errors calculated from the ground truth.

TABLE 11A

| | computed from pairs of reads | | | |
|---|---|---|---|---|
| | | | | pair |
| | match | mismatch | total | error rate |
| disrupted C-18 | 956575411 | 691628 | 957267039 | 0.00072 |
| disrupted CA−26 | 13033464130 | 31977608 | 13065441738 | 0.00245 |
| unmutated C-18 | 103781 | 49602 | 153383 | 0.32339 |
| unmutated CA−26 | 215185682 | 79719814 | 294905496 | 0.27032 |

TABLE 11B

| | computed from read error (synth error removed) | | | | |
|---|---|---|---|---|---|
| | | | | error | pair |
| | on-target | off-target | total | rate | error rate |
| disrupted C-18 | 1612051 | 585 | 1612636 | 0.00036 | 0.00073 |
| disrupted CA−26 | 4312537 | 5442 | 4317979 | 0.00126 | 0.00252 |
| unmutated C-18 | 44531 | 116821 | 161352 | 0.72401 | 0.39964 |
| unmutated CA−26 | 1910332 | 359410 | 2269742 | 0.15835 | 0.26655 |

From the paired agreement error rate when reads are from the same first copy and the paired agreement rate when the paired reads are from different first copies of the same initial template one can compute first round error rate. From those three rates one can estimate the rate of false positives with any number of independent reads of known provenance.

Data from Panels Confirm Low Error Rates Upon Partial Mutagenesis

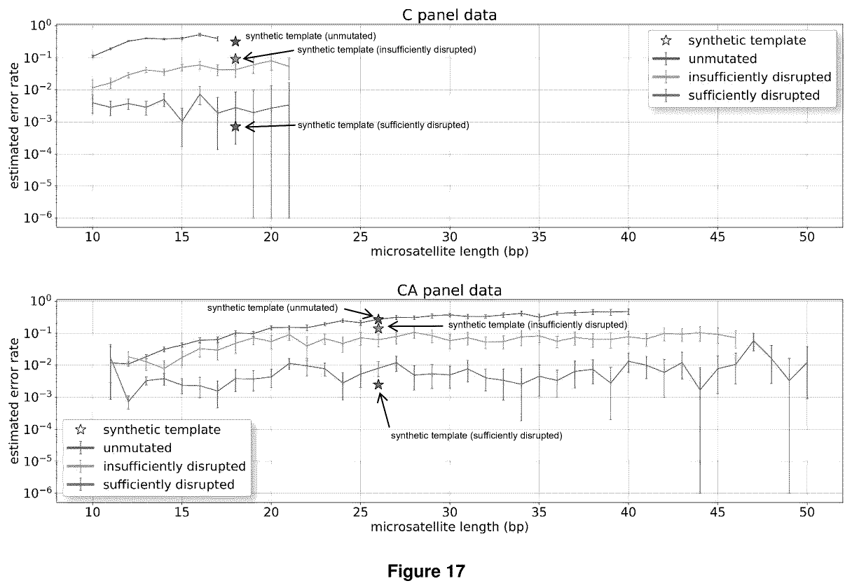

Thousands of reads were obtained from panel-enriched microsatellites of a human cell line. The DNA fragments were end labeled with varietal tags and primer binding sites for amplification, and then the DNA was enriched with the panels described elsewhere in this application. Enriched DNA was then either untreated or treated with bisulfite, and then used to generate multiple first copies of the fragments. These were next amplified to prepare sequencing libraries, and sequenced. Processing of the reads included mapping the reads to the loci of the panel, identifying each read by its template and first copy tags, determining the disruption index as described in other sections of this application, and determining the length of the microsatellite. The data was further analyzed to determine the error rate as a function of initial microsatellite length, both for C- and AC-microsatellites. To do this, how often two reads from the same template molecule agree on the microsatellite length was determined using the method of matched pair read agreement. The results confirm the predictions from the studies on synthetic templates: error rates are several orders of magnitude lower for disrupted templates than for unmutated templates, and the degree of disruption matters. More detail is found in the description of FIG. 17 in the Brief Description of the Drawings.

### Example 10—Directed Partial Mutagenesis

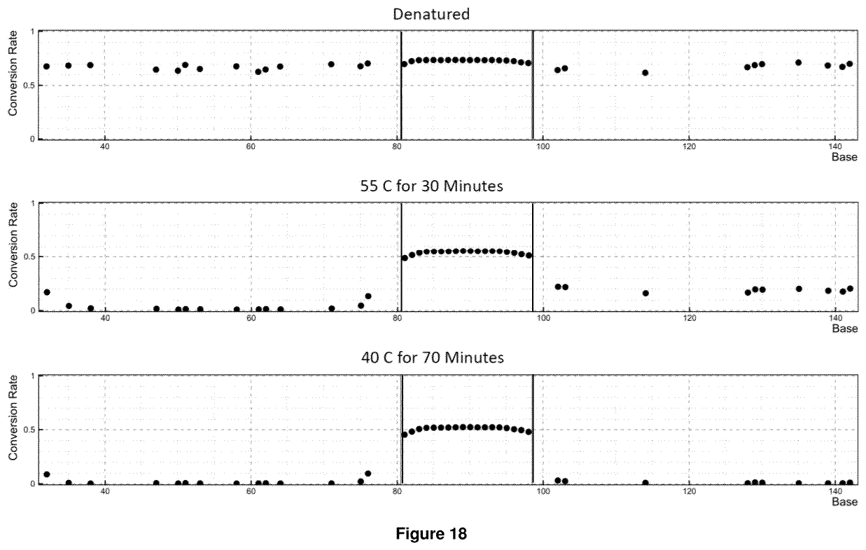

In an optimal scenario, panels would be used to enrich microsatellites in which the microsatellite has been partially but heavily mutagenized, but the flanks used for enrichment have not. In this example, a protocol is provided which demonstrates that directed bisulfite mutagenesis works as predicted (See FIG. 18).

In this example, the inventors designed and tested a synthetic template comprised of a mononucleotide C repeat of 18 bases and two flanks, a left and a right. Blockers, sequences complementary to the flanks, were obtained. We let template and excess blocker molecules anneal, and then templates alone (FIG. 18, panel A), or templates with blockers (FIG. 18, panels B and C) were treated to bisulfite mutagenesis. We used two different treatment protocols, which differed mainly in the temperature of the reaction: 55 degrees centigrade for 30 minutes (FIG. 18, panel B), and 40 degrees for 70 minutes (FIG. 18, panel C). FIG. 18 shows every cytosine in the synthetic template: its position in the template displayed on the X-axis, and the proportion of conversion from C to T on the Y-axis. FIG. 18 shows that the blockers protect the flanks from mutagenesis almost entirely at the lower of the two temperatures.

### Example 11—Eccentricity, a Measures of Microsatellite Length Deviation

By taking a sample from a normal tissue (such as blood), the germline state of the individual is clearly evident at each locus. Typically, there are two cases: (1) there is a single allele length accounting for most of the templates (mono-allelic) or (2) two allele lengths that occur at about 50% (bi-allelic). Allele frequency calculations are adjusted to account for bias due to coverage and likelihood of disruption: shorter microsatellites are more likely to be observed in a read and more likely to be disrupted for the same mutation rate as a longer microsatellite.

Given the germline state at a locus, one can measure the eccentricity of sample to its germline state. For a given locus with germline allele state (A, B) and a frequency distribution of lengths P(l), the K-eccentricity of P with respect to (A, B) is defined as:

$${E}_{K} ( A , B , P ) = {\sum }_{l} P ( l ) min {( \vert A - l \vert , \vert B - l \vert )}^{K}$$

If the sample lengths at a locus are limited to those of the germline, the eccentricity is zero. Otherwise, the larger the weight of a non-germline allele and the greater its distance from the nearest germline allele, the larger the eccentricity value at the locus.

For a panel consisting of many loci, the eccentricity at each locus is computed and these values are summarized by calculating the mean eccentricity over all mono-C and all d-AC loci. The average eccentricity may also be computed for mono-C or di-AC loci after first restricting to loci that exhibit low eccentricity in a normal sample.

### Example 12—Microsatellite Instability (MSI)

A large proportion of tumors are deficient for mismatch-repair and exhibit elevated levels of variation in microsatellite lengths compared to germline. Tumors of this type are said to exhibit “microsatellite instability” and are labeled as MSI-high. Tumors that do no exhibit this increased variation are called MSI-low. Tumor from patients with and without microsatellite instability were examined using the blood as a source of the germline state. In the patients with microsatellite instability, a significant fraction was detected of microsatellite loci exhibiting a dominant length in the tumor that is distinct from the lengths observed in the blood of the patient.

It was observed that the MSI-high patients have tumors with non-germline lengths at 50-80% of mono-C alleles and from 20-40% of di-AC alleles. In contrast, it was observed that MSI-low patients have tumors with non-germline lengths at 5-10% of mono-C alleles and 2-5% of di-AC alleles. In practice, the inventors expect that MSI-high will include non-germline lengths at 20-80% of mono-C alleles and 10-50% of di-AC loci. The inventors expect that MSI-low tumors will include non-germline lengths at less than 15% of mono-C alleles and less than 5% of di-AC loci.

Thus, it was observed that MSI tumors have extreme eccentricity compared to blood and non-MSI tumors, and thus MSL determination and eccentricity forms a method for distinguishing tumor types.

### Example 13—Blood Vs Cell-Free

Although the inventors have not yet measured MSLV in cell-free DNA, the inventors expect that most of the DNA read in cell-free derive from the blood. Samples from both blood cells and the cell-free component of the blood are collected. By comparing the profile from blood cells to the profile from the cell-free, hematopoietic clonality in microsatellite length can be subtracted from the observations in the cell-free component. Any residual eccentricity beyond expectation indicate an expanding clone. For improved capture efficiency, multiplex accurate sensitive quantification (MASQ) protocol is adapted for targeting microsatellites.

### Example 14—Early Detection of MSI Tumors

In silico experiments were performed in which templates were sampled from tumor and blood DNA in different proportions. The eccentricity of the sample compared to the germline variants known in the blood was then computed, as described above. Using a value of K=2, the average eccentricity over all mono-C loci was computed, restricted to loci with low eccentricity in the blood (as in 1). In simulations it was observed that where the tumor proportion equals or exceeds 1 part in 200, the average eccentricity of the mixed sample exceeded the average eccentricity of the blood sample in ten thousand simulations.

### Example 15—Serial Monitoring for Detection

The emergence of a new clone will be best be detected with prior information about the cell-free state of the individual. Following the individual over time will provide the best indication of a new clone. Existing work shows a distribution of allele lengths present in the blood. That distribution is likely to be quasi-stable over time. By serial monitoring of a patient over time, the emergence of a new length in the cell-free component of the blood is distinguished from regular variation, signaling the emergence of a new clone.

### Example 16—Analysis of Microsatellites in Patients with Endometrial Tumors

DNA from tumor and blood from patients with endometrial tumors were collected. Two of the tumors (patients 53 and 55) were classified as microsatellite unstable or “MSI-high.” One of the tumors (patient 61) was classified as microsatellite stable or MSI-low. First, the DNA was fragmented and adapted with mutation resistant primers. Separately, for the blood and the tumor DNA from each patient (6 samples), enriched mutated libraries were prepared as before. The libraries were sequenced on a MiSeq instrument generating between 1 and 2 million reads per sample. Reads were mapped and analyzed as before with measurements determining the length of the microsatellite and if the microsatellite was sufficiently disrupted.

Using only disrupted reads and templates with more than one first copy and a sufficiently high consensus among reads (better than 95%), the template was assigned a microsatellite length and counted. From the blood data, the germline alleles were determined when there was sufficient coverage (20 or more templates in the major allele) and a sample was called heterozygous at a locus if the frequency of the second allele exceeded 40% the coverage of the major allele. These conditions were used and major alleles (homozygous or heterozygous) were determined in both the blood sample and the tumor sample.

TABLE 12

| | mono C | | | di AC | | |
|---|---|---|---|---|---|---|
| | | | tumor | | | tumor |
| | | | alleles | | | alleles |
| pa- | number | germline | absent in | number | germline | absent in |
| tient | of loci | alleles | germline | of loci | alleles | germline |
| 53 | 616 | 981 | 711 | 602 | 825 | 244 |
| 55 | 619 | 1008 | 587 | 590 | 834 | 178 |
| 61 | 603 | 948 | 68 | 519 | 657 | 8 |

The table above is first divided into the 630 mono-C loci and the 630 di-AC loci. Then, for each patient, the table shows the number of loci with sufficient coverage to call a genotype in both the tumor and the normal samples. The next column shows the total number of germline alleles summed over all well covered loci. The last column shows the number of significant tumor alleles that were not present in the germline alleles.

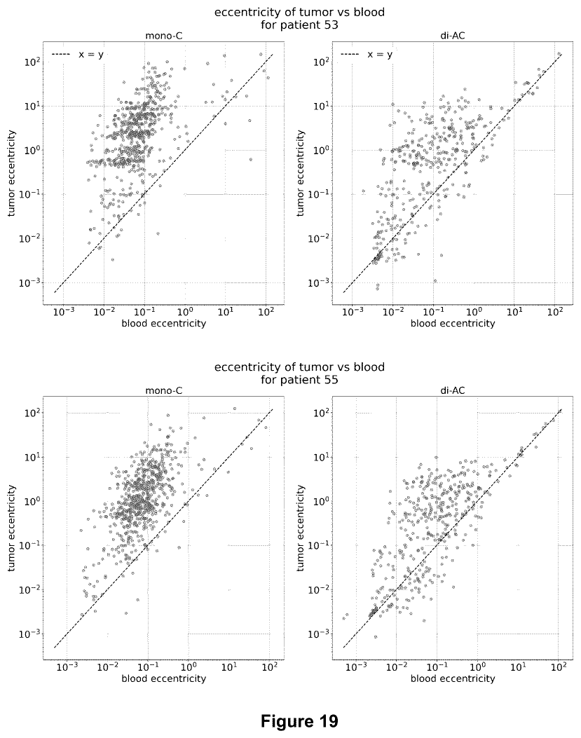

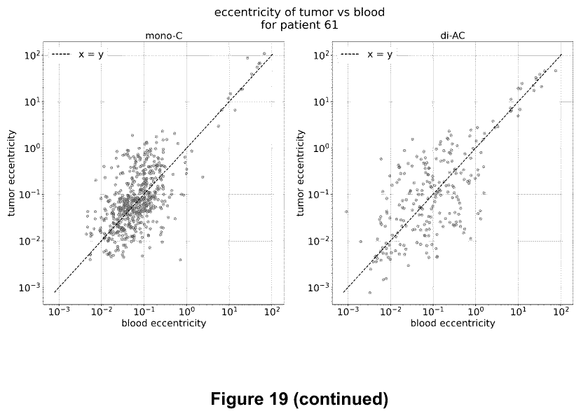

For each locus, the eccentricity in the blood and in the tumor for well-covered loci was also calculated. The plot of tumor vs normal eccentricity are provided in FIG. 19. For both the high-MSI patients (53 and 55) the eccentricity in the tumor exceeded the blood in ˜94% of mono-C loci and ˜62% of di-AC loci. In contrast, the low MSI patient (61), the tumor eccentricity exceeded the blood 51% of the time (practically random) and for the di-AC loci, the tumor eccentricity exceeded the blood 38% of the time.

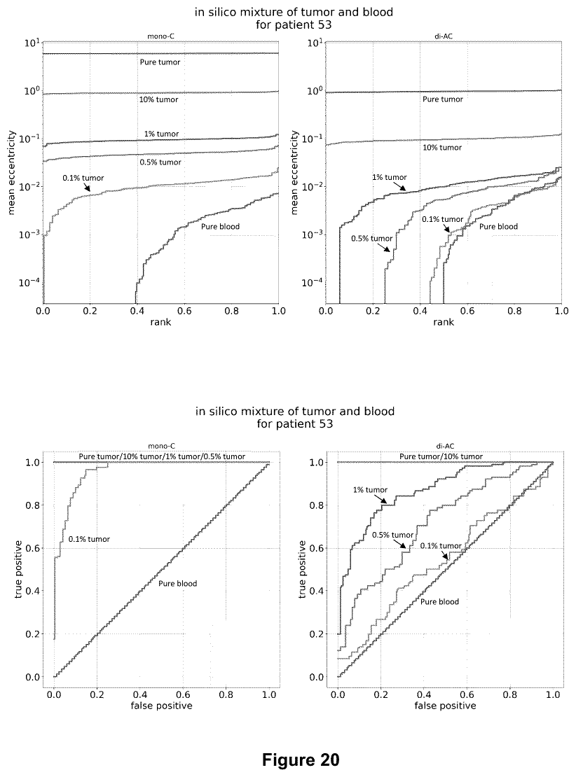

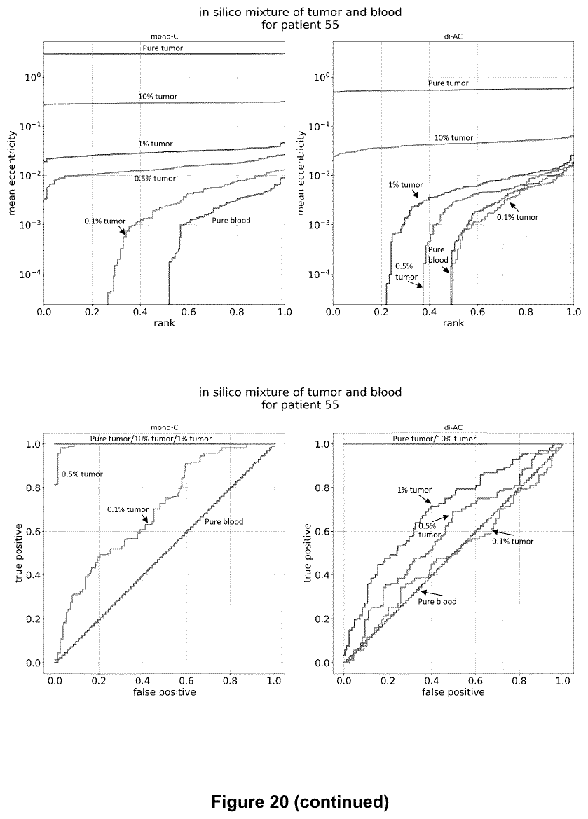

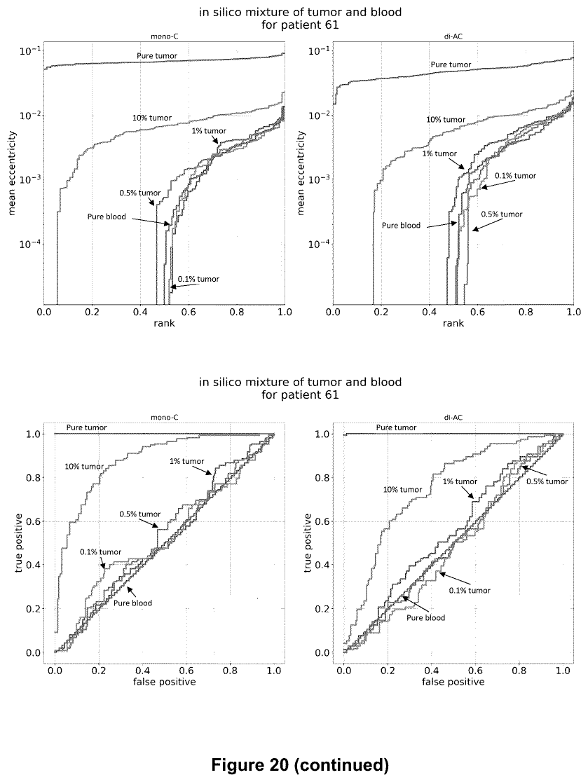

In silico mixing of the blood and tumor samples was performed at various levels to simulate a cell-free profile. The tumor and blood data were sampled with replacement at different ratios while keeping the coverage fixed. The pure blood data sampled only from the blood while the 1% tumor data sampled 1% of its templates from the tumor and 99% from the blood. The pure blood sample was used to identify microsatellites with low hematopoietic instability, restricting to loci where the blood eccentricity was below 1 (as in the plots shown in FIG. 19, these are 97% of mono-C and 94% of di-AC.) The mean eccentricity value for the C and AC for each simulation is then recorded, always using the genotype determined from the blood. 10,000 simulations were performed for each concentration. In the plots shown in FIG. 20, the distribution of mean eccentricity values for the three patients and the accompanying power curves were computed using the pure blood data as the null distribution. These plots show that for high-MSI tumors, one can determine that a mixture containing greater than 0.5% of tumor against a background of 99.5% blood differs significantly from blood alone. With sufficient instability, even 0.1% tumor is measurable with a low false positive rate. In contrast, this approach is not well-powered for low-MSI tumors.

### DISCUSSION

### Template and First Copy Identity

In the first example, in which it was demonstrated that partial mutagenesis reduces replication error of microsatellite length, read redundancy is used to reduce error of length determination. Varietal tags are used to identify initial templates and their multiple individual first copies. Varietal tags are described, for example, in U.S. Pat. No. 9,404,156, the entire contents of which is specifically incorporated herein by reference.

Two varietal tags are incorporated into the synthetic templates, one of the left and one on the right of the sequences that flank the microsatellite tract. A third varietal tag is added by priming during the first-round replication of the synthetic templates. Generally speaking, in that example these varietal tags are identifiers that enable one to aggregate read lengths with the molecule from which they derive. As shown and stated herein, aggregating read length information over a first copy or an initial template reduces error when unanimity or even a consensus of those read lengths is sought. Redundant read coverage over a template thus reduces error in length determination and increases accuracy, but this cannot be accomplished without identity of the initial template or its individual first copies.

Identity of templates and their first copies does not require varietal tags. It can be achieved by other means. For example, the template may have a relatively unique set of end fragment sequences, caused for example by processes of degradation during sample processing that distinguish one template from another. Alternatively, partial mutagenesis applied to the template or its first copies will often create a random and unique pattern of sequence conversion, individually marking that DNA fragment.

### Methods of Mutagenesis

In Example 1 above, bisulfite treatment of the original template is used for partial mutagenesis. It has well-known performance characteristics, in particular the nearly random conversion of C to U, which base pairs with A and so is later read as a T (Kumar, V. et al., 2018; Levy, D. & Wigler, 2014).

However, other methods for partial mutagenesis could be used. For examples, the templates could be methylated, and then treated enzymatically. Or first copies could be mutagenized, either by bisulfite, or enzymatically after incorporation of methyl cytosine into the first copies, or after incorporating other nonstandard nucleotides that cause mutation.

Described herein in more detail is a method for enzymatic mutagenesis with methyl cytosine. In the template replication step, instead of using a mixture of only standard nucleotides, 5-methyl-dCTP is added into the standard nucleotide mixture to achieve a 1:1 ratio of standard dCTP versus 5-methyl-dCTP in the product. The incorporation of methyl-C in the PCR or template replication product is a random process.

Enzymatic Methyl-seq Conversion Module (NEB, E7125S) is used to convert standard cytosine in the PCR template to uracil, while leaving 5-methylcytosine (5mC) unchanged. This comprises two steps. The first step uses the TET2 enzyme to oxidize 5-methylcytosine into 5-carboxycytosine so that it will be protected from being converted by APOBEC. The second step uses the APOBEC enzyme to convert the non-oxidized cytosines to uracils. APOBEC itself has a heavily biased C-to-T conversion rate based on sequence context, so using APOBEC alone will not achieve a random mutation pattern.

When amplifying the APOBEC-treated product, it was discovered that adding 5-methyl-dCTP (same concentration as standard dCTP) into the standard PCR nucleotide mixture will significantly increase the yield for long (5 or 10 kb) products, probably because 5-methyl-dCTP takes slightly larger steric space than standard dCTP and this difference can be used for compensating the space taken by 5-carboxycytosine in the template.

In other implementations nonstandard nucleotides such as inosine can be incorporated into first copies. These will have altered base-pairing activities. The degree of incorporation will determine the degree of mutagenesis. In another implementation, other enzymatic treatments can be used, such as adenine deaminase, which converts adenine directly to inosine in the template. In still another implementation nick translation can be used to alter the nucleotide composition of the template directly by replacement with a nonstandard nucleotide.

### Impact of Partial Mutagenesis on Error Rates

Disruption of the tandem repeat structure reduces its replicative error rate. The degree of disruption, defined as the number of tandem repeat units that have been altered by mutagenesis and the length of the longest remaining tract of repeat units, is directly related to this reduction (FIG. 13). As the extent of mutagenesis is increased, the extent of disruption increases, but only up to a point. As complete mutagenesis is approached, a new tandem repeat can appear replacing the original one, causing a new instability in the replicative error. This is evident in Table 7 and FIG. 10.

### Directed Partial Mutagenesis

In embodiments of the invention, the repeat region of a microsatellite is partially mutagenized but the sequence of one or both of the flanking regions is left intact. In some embodiments of the invention, sample DNA is first enriched, and then mutagenized, and then replicated/amplified. But in some cases, the yield of the initial templates may be increased in an assay if sample DNA is partially mutagenized first and the sample containing mutagenized templates is then replicated/amplified and enriched with flanks from the desired loci. Unfortunately, if mutagenesis changes the flanks, enrichment may be less efficient. However, if the mutagenesis is directed to the repeat structure, and spares the flanks, efficiency of enrichment can be preserved even if sample DNA is partially mutagenized before replication, amplification, and enrichment.

Several techniques can be used to direct mutagenesis, and with bisulfite mutagenesis this is particularly straightforward. Bisulfite mutagenesis requires single stranded templates. Hence for bisulfite mutagenesis, sample DNA is typically melted, and then treated, resulting in the possibility of converting any given C in the sequence to a T. However, if after melting, an excess of synthetic oligonucleotides complementary to the flank sequence is added, a partially double stranded molecule is obtained, with the flank regions being double stranded and the repeat region single stranded. Exposing these partially double stranded partially single stranded DNA molecules to the partial bisulfite mutagenesis protocol results in the partial mutagenesis of the repeat region, but little to no mutagenesis of the flanks.

### Methods of Processing Panel-Based Partial Mutagenesis Sequence Data

Given that the average user who wishes to measure MSLV at loci enriched in panels may not possess the informatics and genomics expertise to process partially mutagenized sequence data, we provide here an example of the database and source code that could accompany a given panel-based kit, customized for that panel and method of mutagenesis.

### Overview

Most sequencing analysis programs begin with a mapper that provides coordinates of a sequence read to a reference genome. Such methods are problematic after partial mutagenesis, since the mutagenized sequence will not match the reference genome. Many potential users will not be familiar with the techniques needed to do that. Existing mutagenized sequence data alignment software is also biased against aligning reads with microsatellites of non-reference length. Moreover, mapping to the entire genome is a waste of compute time and power, given that one cares about only the mapping of reads to a fixed and known number of loci, the loci enriched by the panel. The total number of base pairs of interest are one-ten thousandth or so of the entire genome. Given that the loci of interest are small in number, and we know what can result from a given partial mutagenesis procedure, we design a method of exact matching to a relatively small alignment index database. The mapping to this database, provided with the kit, can be done rapidly on a personal computer with source code supplied with the kit

The method can be broken into three parts:
(a) An alignment index is prepared in advance, custom designed to the panel, and provided to the user;
(b) the alignment index is used to identify pairs of flank sequence matches for each locus; and
(c) from the paired flank matches, the length of the repeat sequence is measured, the repeat sequence is captured, and the disruption indices computed.

### Algorithmic Details:

Panel flank sequences have low complexity, so low that even short subsequences of the flanks immediately adjacent to each microsatellite can be unique within the set of all possible flank sequences, even while allowing for sequence conversion from partial mutagenesis. These short subsequences are recorded in the alignment database in all possible variations that can arise from partial mutagenesis, indexed to the flank from which they arise, and the precise number of bases to the boundary of the repeat sequence itself. The database structure is optimized for exact match search speed (using either a suffix array, a suffix tree, FM index, a burrows wheeler transform, or the like). Preparing this database is done once for each given panel design, and sent to the user.

A fast, unique exact matching algorithm is used to search for matches between each read and the database. If a read has a match to both flanks of a panel locus and does not match both flanks of any other locus, then that read can be confidently assigned to the panel locus matched. If a read matches the control template flanks, then that read can be confidently assigned to a control template. Since the resulting database is orders of magnitude smaller than the entire genome and the matching algorithm is exact, the alignment process is much faster than possible using standard alignment software.

Once a read has one match to a left flank subsequence and one to a right flank subsequence, the coordinates of those matches in the read are known, from which the microsatellite length is readily computed (from the distance to the microsatellite boundary contained in the index), and the microsatellite length start and stop position in the read is known, and its disruption indices are readily computed. The output data is then associated with the locus or control, as well as the template, first copy tags and/or sample tags. From this data the distribution of lengths over the various provenances can be computed, and statistical inferences made.

### General Disruption Indices

It is evident from experiments involving synthetic templates and panel-enriched microsatellite loci that the degree of disruption determines error rates. Such error rates can be conveniently measured in pairs of reads from the same template, or in pairs of reads from different first copies of the same initial templates, as seen in FIG. 17.

For the examples described herein, a combination of two disruption indices were used: setting a threshold for the proportion of C converted to T in the microsatellite; and setting a threshold for the maximum length of a repeated sequence remaining in the mutagenized microsatellite. However, any number of other disruption indices can be made, and any method for quantifying or thresholding the degree of disruption of a repeat structure are referred to herein as a generalized disruption index. Another such index, called the k-mer trace, is described here to illustrate some the structural features of a disrupted microsatellite that might be pertinent to its error rate.

The k-Mer Trace

Let S be the set of k-mers in the microsatellite. For this example, set k=5. For each 5-mer in S, compute as follows: For each instance of two exact k-mer matches in the microsatellite determine 1/d″ where d is the distance in bases of that match within the satellite, and n is a parameter that can be set as desired. Summing over all such matches gives us the “k-mer trace.” The intuition behind this measure is that the likelihood of a polymerase slipping during a replication through a repeat sequence is related to the likelihood that nearby there is an exact match k bases long for the sequence just replicated.

### Degree of Disruption and Error Rate

There are many choices for disruption indices, and parameters to choose in setting these, and for determining thresholds. In this section a general approach to using any given disruption index is provided. For any such index, one can use the method illustrated in Example 9 and FIG. 17, the method of matched pair read agreement, to determine the error rates for those reads. Note that to apply this method to sequence data, it is necessary to have at least some identity for the template and ideally for first copies of those templates, so that matched read pairs can be found. Also note that the selection of parameters and thresholds can be chosen for each specific microsatellite length and type.

### Kits

Standardized kits can be used to assess microsatellite lengths for a given set of microsatellites and a given mutagenesis protocol. The important elements of those kits will be customized components, while other components may be standardized reagents purchased from vendors. The important elements will include: (a) panels for enrichment, based on either the microsatellite flanks or the microsatellite sequence itself; (b) control templates to determine yield, degree of mutagenesis, and error rates based on degree of disruption, which control templates will match the panels and method of mutagenesis; and (c) oligonucleotide adaptors for conferring identities and replication primer binding sites and primers, which adaptors will match control templates and method of mutagenesis. A kit may also comprise oligonucleotides complementary to the flanking portions of microsatellites to protect the flanking portions from partial mutagenesis to facilitate directed partial mutagenesis as described above.

Below is an example of a complete kit.

### Kit Based on Bisulfite Mutagenesis

The following kit is designed and compiled for genomic DNA as starting material undergoing panel enrichment and bisulfite treatment (FIG. 11).

In step 1, genomic DNA is fragmentated, end polished, and 3′-dA tailed by NEBNext® Ultra™ II FS DNA Library Prep Kit for Illumina (NEB). As this kit combines all the reagents of the above three reactions, enabling these steps to be performed in the same tube without clean-ups, it avoids the sample loss and reduces bench time.

Double-stranded synthetic control templates are spiked-in each sample after step 1. These control templates have varietal tags, flanking sequences that can be captured by panels, template and sample barcodes; and universal primers. These control templates are used to monitor yield of the entire process.

Fish-tail adapters are ligated to the dsDNA (see below for details). They are designed to contain universal primers, varietal tags, and sample barcodes, which are resistant to bisulfite conversion.

In step 3, DNA fragments containing target MS loci are enriched by panels. Blockers are added to the capture solution to enhance the specificity of target enrichment by blocking non-specific hybridization between fish-tail adapter sequences.

Single-stranded synthetic control templates (I) are spiked in just before the mutagenesis step. This collection of control templates have microsatellites of known length and composition to match the panel and mutagenesis protocol, and flanking sequences that identify and delineate them. They are used to assess the efficacy of the disruption and its effects on replicative error rates, and enable the production of Look-up tables.

Enriched DNA fragments are partially bisulfite converted by EZ DNA Methylation-Direct Kit (Zymo Research) (step 4). This kit is chosen because it is designed to minimize template degradation and the loss of DNA during treatment and clean-up.

Additional single-stranded synthetic control templates (II) are added. This collection of control templates contain microsatellites with known disruptions. They are used in the production of the Look-up tables.

By using a primer containing one of the two sequencing primers (read 1 primer, P5), a varietal tag, and up2 (step 5), the first-round copies are generated via multiple cycles of linear amplification in NEBNext Q5U Master Mix (NEB). This master mix contains modified Q5® High Fidelity DNA Polymerase, optimized for amplification of uracil-containing templates. Double-stranded DNA fragments are obtained by another round of linear amplification using a primer containing the second sequencing primer (read 2 primer, P7) and up1 (step 6).

After size selection for the length range from 300 bp to 500 bp, exponential amplification in NEBNext Ultra II Q5 Master Mix (NEB) is carried out to make the final sequencing libraries (step 7). All libraries are prepared with indexed sample primers to enable pooling of samples

### FISH-TAIL Adaptors

Two oligonucleotides are designed to prepare a fish-tail primer. The first oligonucleotide contains a universal primer binding site, a varietal tag, and a sample barcode, while the second contains a different universal primer binding site and a sequence complementary to the above sample barcode. These two oligonucleotides are annealed to each other via the sample barcode sequences to form fish-tail primers with a 3′ thymine overhang. These fish-tail primers are different from those adaptors used in duplex sequencing which do not have sample barcodes and are difficult to prepare. Fish-tail primers have the following four properties: (1) the initial templates are tagged with varietal tags to facilitate the error rate estimations; (2) each of the modified templates has distinct universal primer binding sites at 5′ and 3′ ends, respectively, so linear replication can be applied to these templates to generate first copies; (3) modified initial templates, including a unique sample barcode for each sample, can be pooled before enrichment to increase the efficiency; (4) the universal primer binding sites are resistant to mutagenesis, which maintains the high efficiency of replication or amplification after the process of mutagenesis.

### Synthetic Control Templates

Control templates are used to monitor the performance features of the process: yield, degree of mutagenesis, and replication error rates. The double stranded control templates have sequences that result in enrichment by the panel, and “private” universal primers so they can be assayed throughout the process to monitor yield. The single stranded control molecules are added before (I) and after (II) mutagenesis. Those added before contain microsatellites. Those added after contain microsatellites with specified disruptions. Their flanks distinguish them from other templates. Their purpose is to provide measures of replication error. Both the single and double stranded control molecules have varietal tags for counting, and their precise sequence is matched to the methods of mutagenesis and the panels.

### Flanking Sequences and Provenance

Useful templates containing microsatellites, whether synthetic of biological origin, have flanking sequences that delineate the microsatellites. The flanking sequences are typically modified, either by ligation or by primer extension to include useful primer binding sites and identifiers. The distance between the flanking sequences is used to measure the microsatellite length. But more than that they define the provenance of the templates. For example, the flanks identify the microsatellite locus in the genome if the template originated from a biological sample, or as a synthetic template that may have been added as a control. Additionally, varietal tags may identify initial template molecules, or first copies of them. Sample barcodes are added and needed if samples are pooled.

### BIBLIOGRAPHY

Avvaru, A. K., Sharma, D., Verma, A., Mishra, R. K. & Sowpati, D. T. MSDB: a comprehensive, annotated database of microsatellites. Nucleic Acids Res 48, D155-D159, doi: 10.1093/nar/gkz886 (2020).
Bacher, J. W. et al. Development of a fluorescent multiplex assay for detection of MSI-High tumors. Dis Markers 20, 237-250, doi: 10.1155/2004/136734 (2004).
Boland, C. R. et al. A National Cancer Institute Workshop on Microsatellite Instability for cancer detection and familial predisposition: development of international criteria for the determination of microsatellite instability in colorectal cancer. Cancer Res 58, 5248-5257 (1998).
Bonneville, R. et al. Landscape of Microsatellite Instability Across 39 Cancer Types. JCO Precis Oncol 2017, 1-15, doi: 10.1200/PO.17.00073 (2017).
Bonneville, R. et al. Landscape of microsatellite instability across 39 cancer types. JCO precision oncology 1, 1-15 (2017).
Brouwer, J. R., Willemsen, R. & Oostra, B. A. Microsatellite repeat instability and neurological disease. Bioessays 31, 71-83, doi: 10.1002/bies.080122 (2009).
Clarke, L. A., Rebelo, C. S., Goncalves, J., Boavida, M. G. & Jordan, P. PCR amplification introduces errors into mononucleotide and dinucleotide repeat sequences. Mol Pathol 54, 351-353, doi: 10.1136/mp.54.5.351 (2001).
Eshleman, J. R. & Markowitz, S. D. Mismatch repair defects in human carcinogenesis. Hum Mol Genet 5 Spec No, 1489-1494, doi: 10.1093/hmg/5.supplement_1.1489 (1996).
Fujimoto, A. et al. Comprehensive analysis of indels in whole-genome microsatellite regions and microsatellite instability across 21 cancer types. Genome Res 30, 334-346, doi: 10.1101/gr.255026.119 (2020).
Fungtammasan, A. et al. Accurate typing of short tandem repeats from genome-wide sequencing data and its applications. Genome Res 25, 736-749, doi: 10.1101/gr.185892.114 (2015).
Georgiadis, A. et al. Noninvasive Detection of Microsatellite Instability and High Tumor Mutation Burden in Cancer Patients Treated with PD-1 Blockade. Clin Cancer Res 25, 7024-7034, doi: 10.1158/1078-0432.CCR-19-1372 (2019).
Goriely, A. & Wilkie, A. O. Paternal age effect mutations and selfish spermatogonial selection: causes and consequences for human disease. Am J Hum Genet 90, 175-200, doi: 10.1016/j.ajhg.2011.12.017 (2012).
Hause, R. J., Pritchard, C. C., Shendure, J. & Salipante, S. J. Classification and characterization of microsatellite instability across 18 cancer types. Nat Med 22, 1342-1350, doi: 10.1038/nm.4191 (2016).
Hause, R. J., Pritchard, C. C., Shendure, J. & Salipante, S. J. Classification and characterization of microsatellite instability across 18 cancer types. Nature medicine 22, 1342-1350 (2016).
Highnam, G. et al. Accurate human microsatellite genotypes from high-throughput resequencing data using informed error profiles. Nucleic Acids Research 41, e32-e32, doi: ARTN e3210.1093/nar/gks981 (2013).
Jaiswal, S. & Ebert, B. L. Clonal hematopoiesis in human aging and disease. Science 366, doi: 10.1126/science.aan4673 (2019).
Kim, T. M., Laird, P. W. & Park, P. J. The landscape of microsatellite instability in colorectal and endometrial cancer genomes. Cell 155, 858-868, doi: 10.1016/j.cell.2013.10.015 (2013).
Kim, T.-M., Laird, P. W. & Park, P. J. The landscape of microsatellite instability in colorectal and endometrial cancer genomes. Cell 155, 858-868 (2013).
Kumar, V. et al. Partial bisulfite conversion for unique template sequencing. Nucleic Acids Res 46, e10, doi: 10.1093/nar/gkx1054 (2018).
Kunkel, T. A. Frameshift mutagenesis by eucaryotic DNA polymerases in vitro. J Biol Chem 261, 13581-13587 (1986).
Lai, Y., Shinde, D., Arnheim, N. & Sun, F. The mutation process of microsatellites during the polymerase chain reaction. J Comput Biol 10, 143-155, doi: 10.1089/106652703321825937 (2003).
Levy, D. & Wigler, M. Facilitated sequence counting and assembly by template mutagenesis. Proc Natl Acad Sci USA 111, E4632-4637, doi: 10.1073/pnas. 1416204111 (2014).
Loman, N. J. et al. Performance comparison of benchtop high-throughput sequencing platforms. Nat Biotechnol 30, 434-439, doi: 10.1038/nbt.2198 (2012).
Luria, S. E. & Delbrück, M. Mutations of bacteria from virus sensitivity to virus resistance. Genetics 28, 491 (1943).
Lynch, H. T. et al. Review of the Lynch syndrome: history, molecular genetics, screening, differential diagnosis, and medicolegal ramifications. Clin Genet 76, 1-18, doi: 10.1111/j.1399-0004.2009.01230.x (2009).
Middha, S. et al. Reliable Pan-Cancer Microsatellite Instability Assessment by Using Targeted Next-Generation Sequencing Data. JCO Precis Oncol 2017, 1-17, doi: 10.1200/PO.17.00084 (2017).
Minoche, A. E., Dohm, J. C. & Himmelbauer, H. Evaluation of genomic high-throughput sequencing data generated on Illumina HiSeq and genome analyzer systems. Genome Biol 12, R112, doi: 10.1186/gb-2011-12-11-r112 (2011).
Moffitt, A. B. et al. Multiplex accurate sensitive quantitation (MASQ) with application to minimal residual disease in acute myeloid leukemia. Nucleic Acids Res 48, e40, doi: 10.1093/nar/gkaa090 (2020).
Murphy, K. M. et al. Comparison of the microsatellite instability analysis system and the Bethesda panel for the determination of microsatellite instability in colorectal cancers. J Mol Diagn 8, 305-311, doi: 10.2353/jmoldx.2006.050092 (2006).
Nakamura, K. et al. Sequence-specific error profile of Illumina sequencers. Nucleic Acids Res 39, e90, doi: 10.1093/nar/gkr344 (2011).
Ranum, L. P. & Day, J. W. Dominantly inherited, non-coding microsatellite expansion disorders. Curr Opin Genet Dev 12, 266-271, doi: 10.1016/s0959-437x(02) 00297-6 (2002).
Shi, J. et al. Discovery of cancer drug targets by CRISPR-Cas9 screening of protein domains. Nature biotechnology 33, 661-667 (2015).
Shinde, D., Lai, Y., Sun, F. & Arnheim, N. Taq DNA polymerase slippage mutation rates measured by PCR and quasi-likelihood analysis: (CA/GT) n and (A/T) n microsatellites. Nucleic Acids Res 31, 974-980, doi: 10.1093/nar/gkg178 (2003).
Silveira, A. B. et al. High-Accuracy Determination of Microsatellite Instability Compatible with Liquid Biopsies. Clin Chem 66, 606-613, doi: 10.1093/clinchem/hvaa013 (2020).
Snell, R. G. et al. Relationship between trinucleotide repeat expansion and phenotypic variation in Huntington's disease. Nature genetics 4, 393-397 (1993).
Stoler, N. & Nekrutenko, A. Sequencing error profiles of Illumina sequencing instruments. NAR Genom Bioinform 3, 1qab019, doi: 10.1093/nargab/1qab019 (2021).
Verkerk, A. J. et al. Identification of a gene (FMR-1) containing a CGG repeat coincident with a breakpoint cluster region exhibiting length variation in fragile X syndrome. Cell 65, 905-914, doi: 10.1016/0092-8674(91) 90397-h (1991).
Zavodna, M., Bagshaw, A., Brauning, R. & Gemmell, N. J. The accuracy, feasibility and challenges of sequencing short tandem repeats using next-generation sequencing platforms. PLOS One 9, e113862, doi: 10.1371/journal.pone.0113862 (2014).
Abbosh, C. et al. Phylogenetic ctDNA analysis depicts early-stage lung cancer evolution. Nature 545, 446 (2017).
Coombes, R. C. et al. Personalized detection of circulating tumor DNA antedates breast cancer metastatic recurrence. Clinical Cancer Research, clincanres. 3663.2018 (2019).
Cristiano, S. et al. Genome-wide cell-free DNA fragmentation in patients with cancer. Nature, 1 (2019).
Garcia-Murillas, I. et al. Mutation tracking in circulating tumor DNA predicts relapse in early breast cancer. Science translational medicine 7, 302ra133-302ra133 (2015).
Phallen, J. et al. Direct detection of early-stage cancers using circulating tumor DNA. Science translational medicine 9, eaan2415 (2017).
Tie, J. et al. Circulating tumor DNA analysis detects minimal residual disease and predicts recurrence in patients with stage II colon cancer. Science translational medicine 8, 346ra392-346ra392 (2016).

1. A method for measuring microsatellite lengths of initial nucleic acid templates each of which comprise a microsatellite and two flanking portions, which flanking portions delineate the microsatellite, the method comprising:
(a) generating partially mutagenized templates by:
(i) partial mutagenesis of the initial nucleic acid templates;
(ii) partial mutagenesis during production of first copies of the initial nucleic acid templates; and/or
(iii) generating first copies of the initial nucleic acid templates followed by partial mutagenesis of the first copies of the initial nucleic acid templates;
(b) making a sequencing library from the partially mutagenized templates;
(c) sequencing the library from step (b) to generate sequence reads;
(d) selecting a set of sequence reads in which the microsatellite
(i) has been disrupted by mutagenesis so that:
(1) at least two, preferably more of the tandem repeat units in the microsatellite have been mutagenized; and
(2) the microsatellite in the sequence read has no more than 7, preferably 5 or fewer, tandem repeats in a row; or
(ii) exceeds a disruption index so that the error rate between matched pairs is less than 2%, less than 1%, or preferably less than 0.5%, wherein matched pairs are independent reads that share a template, first copy, or locus; and
(e) measuring microsatellite lengths from the set of sequence reads selected in step (d), wherein the measurement is the distance between the flanking portions that delineate the microsatellite of the initial templates.

2. (canceled)

3. The method of claim 1, wherein the partial mutagenesis comprises:
(a) chemical mutagenesis;
(b) enzymatic mutagenesis; or
(C) incorporating nonstandard nucleotides during a step of replication.

4. The method of claim 1, wherein the partial mutagenesis comprises:
(a) treating the initial nucleic acid template or a first copy of the initial nucleic acid template with an enzyme that deaminates nucleotides, preferably wherein the enzyme that deaminates nucleotides is adenine deaminase;
(b) deamination of cytosines, preferably deamination of cytosines by bisulfite mutagenesis;
(c) nick translation of the initial nucleic acid template or a first copy of the initial nucleic acid template to replace nucleotides of the template with nonstandard nucleotides having altered base-pairing activity;
(d) copying the initial nucleic acid templates or first copies of the initial nucleic acid templates in the presence of a mixture of standard nucleotides and nonstandard nucleotides to generate copies comprising standard and nonstandard nucleotides, wherein the nonstandard nucleotides have altered base-pairing activity, preferably wherein the nonstandard nucleotides are deoxyinosine triphosphate;
(e) the steps of:
(i) copying the initial nucleic acid templates or first copies of the initial nucleic acid templates in the presence of a mixture of standard nucleotides and nonstandard nucleotides to generate copies comprising standard and nonstandard nucleotides; and
(ii) subjecting the copies comprising standard and nonstandard nucleotides to a chemical or enzymatic treatment that alters the base-pairing activity of a standard nucleotide without altering the base-pairing activity of its corresponding nonstandard nucleotide,
preferably wherein:
(1) the nonstandard nucleotides are 5-methylcytosine, preferably wherein chemical or enzymatic treatment comprises using a TET2 enzyme to oxidize 5-methylcytosine into 5-carboxycytosine;
(2) the chemical or enzymatic treatment comprises using an APOBEC enzyme to convert cytosines to uracils;
preferably wherein:
(a) the partial mutagenesis is followed by production of first copies in the presence of a mixture of 5-methyl-dCTP and standard nucleotides, preferably wherein 5-methyl-dCTP is present in the mixture at about the same concentration as dCTP; or
(f) the steps of:
(i) copying the initial nucleic acid templates or first copies of the initial nucleic acid templates in the presence of a mixture of standard nucleotides and nonstandard nucleotides to generate copies comprising standard and nonstandard nucleotides; and
(ii) subjecting the copies comprising standard and nonstandard nucleotides to a chemical or enzymatic treatment that alters the base-pairing activity of the nonstandard nucleotide without altering the base-pairing activity of its corresponding standard nucleotide.

5. The method of claim 1, comprising a step of protecting the flanking portions of the initial nucleic acid templates or first copies of the initial nucleic acid templates from partial mutagenesis, preferably wherein protecting the flanking portions of the initial nucleic acid templates or first copies of the initial nucleic acid templates comprises using an excess of oligonucleotides complementary to the flanking portions to protect the flanking portions from partial mutagenesis, preferably wherein the partial mutagenesis comprises deamination of cytosines, preferably deamination of cytosines by bisulfite mutagenesis.

6. The method of claim 1, wherein the initial nucleic acid templates:
(a) are from a biological sample, preferably wherein the biological sample is:
(i) from a tissue biopsy;
(ii) from blood or a blood product;
(iii) from excreta, preferably urine or fecal matter; or
(iv) sputum;
(b) are copies of nucleic acids from a biological sample, preferably wherein the biological sample is:
(i) from a tissue biopsy;
(ii) from blood or a blood product;
(iii) from excreta, preferably urine or fecal matter; or
(iv) sputum; or
(c) are synthetic templates, preferably wherein the synthetic templates each comprise two flanking portions and a microsatellite of known composition n and length, each optionally comprising a varietal tag, a sample barcode, and/or a universal primer binding site, wherein the microsatellite:
(i) comprises nucleotides susceptible to being altered by the step of partial mutagenesis; or
(ii) comprises a known pattern of mutation;
(d) were prepared by random fragmentation of nucleic acids, preferably wherein the random fragmentation was by:
(i) a natural process, preferably degradation of nucleic acids;
(ii) shearing; and/or
(iii) endonucleases,
(e) were prepared by restriction endonuclease cleavage of nucleic acids;
(f) are in a sample that has been enriched for microsatellites by a panel comprising oligonucleotides with sequence complementarity to:
(i) one or more microsatellite flanking portions; or
(ii) a microsatellite repeat motif;
(g) are in a sample enriched for microsatellites and the method comprises a step of enriching a sample comprising a population of nucleic acid templates for microsatellites using a panel comprising oligonucleotides with sequence complementarity to:
(i) one or more microsatellite flanking portions; or
(ii) a microsatellite repeat motif;
(h) comprise one or more adaptors, preferably wherein the one or more adaptors convey template identity and/or sample identity, more preferably wherein the one or more adaptors comprise one or more or all of the following:
(i) a varietal tag;
(ii) a sample barcode;
(iii) a universal primer binding site;
(iv) a purification moiety, preferably biotin; and
(v) a sequencing primer binding site more preferably wherein the one or more adaptors:
(1) consist of nucleotides that are:
(i) not susceptible to being altered in the step of partial mutagenesis; or
(ii) complementary to nucleotide that are not susceptible to being altered in the step of partial mutagenesis;
(2) are added to the initial nucleic templates, first copies of the initial nucleic acid templates, and/or partially mutagenized templates by:
(i) ligation; or
(ii) primer extension; and/or
(3) have a fish-tail structure.

7. (canceled)

8. The method of any one of claims 1 to 7, wherein:
(a) step (a) comprises a step of enriching partially mutagenized templates for microsatellites using a panel comprising oligonucleotides with sequence complementarity to:
(i) one or more microsatellite flanking portions; or
(ii) a microsatellite repeat motif;
preferably wherein the panel is:
(1) a panel of hybridization capture probes; or
(2) a panel of primers to initiate replication; and/or
(b) step (a), part (i) comprises generating first copies of the partially mutagenized templates, preferably wherein one or more adaptors are added to the first copies, more preferably wherein the one or more adaptors comprise one or more or all of the following:
(i) a varietal tag;
(ii) a sample barcode;
(iii) a universal primer binding site; and
(iv) a purification moiety, preferably biotin.

9. The method of claim 1, wherein the initial nucleic acid templates and/or first copies thereof comprise one or more individual identifiers, preferably wherein the one or more individual identifiers comprise:
(a) fragment end sequences if the initial nucleic acid template is from a biological sample and is randomly fragmented;
(b) fragment end sequences if the initial nucleic acid template was prepared using random fragmentation;
(c) a mutational pattern caused by the step of partial mutagenesis, wherein the step of partial mutagenesis is partial random mutagenesis;
(d) a varietal tag attached to the initial nucleic acid templates or first copies of the initial nucleic acid templates;
(e) a sequence of the flanking portions of the microsatellite, which:
(i) specify the locus of the microsatellite in a reference genome if the initial nucleic acid template is from a biological sample; or
(ii) specify a synthetic nucleic acid molecule if the initial nucleic acid template is a synthetic template; or
(f) any combination of the above.

10-12. (canceled)

13. The method of claim 1, wherein step (b) comprises amplification of the partially mutagenized templates, preferably wherein amplification comprises linear amplification, exponential amplification, or both;
more preferably wherein amplification is with:
(i) a DNA polymerase;
(ii) a RNA polymerase; or
(iii) a reverse transcriptase.
even more preferably wherein:
(1) amplification is with primers consisting of nucleotides that are not susceptible to being altered in the step of partial mutagenesis;
(2) amplification is by polymerase chain reaction (PCR); and/or
(3) step (b) comprises:
(a) end-polishing, A-tailing, and sequencing adaptor ligation; and/or
(b) enriching the partially mutagenized templates for microsatellites, before or after amplification, preferably wherein enriching is with a panel comprising oligonucleotides with sequence complementarity to:
(i) one or more microsatellite flanking portions; or
(ii) a microsatellite repeat motif.
preferably wherein the panel is:
(1) a panel of hybridization capture probes; or
(2) a panel of primers to initiate replication;
optionally wherein the partially mutagenized templates comprise a purification moiety and step (b) comprises purifying the partially mutagenized templates using the purification moiety, preferably prior to a step of exponential amplification, preferably wherein the purification moiety is biotin and the partially mutagenized templates are purified by binding of the purification moiety to streptavidin.

14-16. (canceled)

17. The method of claim 1, wherein:
(a) the sequence reads:
(i) are single reads or paired end reads;
(ii) have sample barcode sequences; and/or
(iii) have varietal tag sequences; and/or
(b) the microsatellites:
(i) are at least four repeat units in length;
(ii) comprise repeat units, each of which is no more than 10 nucleotides;
(iii) are at least 12 nucleotides in length;
(iv) are mononucleotide tracts, preferably mono-C tracts;
(v) are dinucleotide tracts, preferably C/G tracts or C/A tracts;
(vi) comprise cytosines;
(vii) comprise adenines;
(viii) are susceptible to a method of partial mutagenesis;
(ix) are known to have unstable replication;
(x) are more than 5 repeat units in length, more than 7 repeat units in length, more than 10 repeat units in length, more than 15 repeat units in length, more than 20 repeat units in length, more than 30 repeat units in length, between 6 and 70 repeat units in length, between 6 and 32 repeat units in length or between 12 and 64 repeat units in length; and/or
(xi) are from a genome of an organism and adjoin flanking portions in the genome of the organism, wherein a flanking portion together with the microsatellite map uniquely to the genome to define the locus of the microsatellite, and wherein the flanking portions delineate the length of the microsatellite.

18. (canceled)

19. The method of claim 1, comprising establishing one or more provenances of the sequence reads, preferably wherein the one or more provenances are:
(a) a locus in a reference genome and the provenance is established using a sequence in one or both of the flanking portions;
(b) a synthetic nucleic acid template and the provenance is established using a sequence in one or both of the flanking portions;
(c) an initial nucleic acid template with a specific individual identifier and the provenance is established using said individual identifier;
(d) a first copy of an initial nucleic acid template with a specific individual identifier and the provenance is established using said individual identifier;
(e) a partially mutagenized template with a specific individual identifier and the provenance is established using said individual identifier;
(f) a partially mutagenized first copy of an initial nucleic acid template with a specific individual identifier and the provenance is established using said individual identifier;
(g) a sample with a specific sample barcode and the provenance is established using said sample barcode; and/or
(h) a partially mutagenized template with a specific degree of microsatellite disruption preferably wherein the provenance is established based a common maximum repeat length and/or a common proportion of mutagenized bases.
preferably wherein the individual identifier comprises:
(iii) fragment end sequences if the initial nucleic acid template is from a biological sample and is randomly fragmented;
(ii) fragment end sequences if the initial nucleic acid template was prepared using random fragmentation;
(iii) a mutational pattern caused by the step of partial mutagenesis, wherein the step of partial mutagenesis is partial random mutagenesis;
(iv) a varietal tag attached to the initial nucleic acid templates or first copies of the initial nucleic acid templates;
(v) a sequence of the flanking portions of the microsatellite, which
(1) specify the locus of the microsatellite in a reference genome if the initial nucleic acid template is from a biological sample; or
(2) specify a synthetic nucleic acid molecule if the initial nucleic acid template is a synthetic template; or
(vi) any combination of the above.

20. The method of claim 1, further comprising generating a distribution of microsatellite read lengths by counting the number of microsatellites of a given length across all measured microsatellite lengths in a set of sequence reads having a shared provenance, wherein the shared provenance is selected from the group consisting of:
(a) sample;
(b) locus;
(c) synthetic template;
(d) initial template identity;
(e) first copy identity; or
(f) degree of disruption;
preferably comprising generating a distribution of consensus microsatellite lengths, wherein:
(i) the consensus microsatellite lengths derive from the distribution of microsatellite read lengths over a set of identified templates by applying a consensus rule;
(ii) the consensus microsatellite lengths derive from the distribution of microsatellite read lengths over a set of identified first copies by applying a consensus rule; or
(iii) the consensus microsatellite lengths derive from a distribution of consensus microsatellite lengths over a set of identified first copies sharing a set of identified initial nucleic acid templates by applying a consensus rule;
more preferably wherein the consensus rule is chosen from the group consisting of:
(i) a unanimity rule, in which the consensus microsatellite length is the only microsatellite length in the distribution and all other microsatellite lengths have a count of zero;
(ii) a plurality rule, in which the consensus microsatellite length is the most common microsatellite length in the distribution;
(iii) the majority P-rule, in which the consensus microsatellite length is the microsatellite length with a count that is greater than or equal to N×P where N is the total number of microsatellite lengths and P is greater than or equal to 0.5;
optionally wherein:
(i) the sequencing library is enriched for microsatellites by a panel comprising oligonucleotides with sequence complementarity to one or more microsatellite flanking portions at one or more loci and optionally comprises synthetic nucleic acid templates of known microsatellite composition, length, and degree of disruption; and
(ii) distributions of microsatellite lengths at one or more loci are generated by:
(1) using an exact matching algorithm to identify matches between the sequence reads and an alignment index database, wherein the alignment index database comprises, for each locus corresponding to a microsatellite of the panel and for each synthetic nucleic acid template, if present:
(1) a subsequence for each flanking portion and all possible variations that can arise from partial mutagenesis; and
(2) the distance of each said subsequence to the microsatellite;
(2) for each sequence read that matches a subsequence of the alignment index database measuring the microsatellite length using the distance in the alignment index database; and
(3) counting the number of microsatellites of a given length across all measured microsatellite lengths in a set of sequence reads having a shared provenance, wherein the shared provenance is selected from the group consisting of:
(1) sample;
(2) locus;
(3) synthetic template;
(4) initial template identity;
(5) first copy identity; and/or
(6) degree of disruption.

21-22. (canceled)

23. The method of claim 1, wherein the step of selecting a set of sequence reads comprises:
(a) using a look-up table of disruption and error rates to estimate the replication error associated with a given pattern of disruption in a sequence read;
(b) selecting sequence reads based on the estimated replication error.

24-25. (canceled)

26. The method of claim 1, comprising estimating a confidence interval for the proportion of initial templates with a microsatellite length L at a single locus in a single sample, wherein the confidence interval is estimated using the distribution of measured microsatellite lengths in reads with a given degree of disruption and a look-up table of disruption and error rates, preferably wherein:
(a) the look-up table of disruption and error rates was generated using synthetic templates that match the microsatellite tracts of the initial templates in composition and length; or
(b) the look-up table of disruption and error rates was generated by:
(i) preparing distributions of read lengths and consensus read lengths of synthetic nucleic acid templates of known microsatellite composition, length, and degree of disruption;
(ii) from the distributions of read lengths and consensus read lengths, estimating an error rate per round of replications as a function of the degree of disruption; and
(iii) from the estimated error rate per round of replication, generating a look-up table of the moments of error as a function of the degree of disruption and number of rounds of replication;
more preferably wherein the confidence interval is used:
(a) to determine the profile of microsatellite length variation in a tumor;
(b) to genotype an individual;
(c) to detect disease loci;
(d) for early detection of cancer; or
(e) to determine the health of a sampled tissue.

27. (canceled)

28. A method of comparing two or more samples over one or more microsatellite loci for microsatellites of length L comprising:
(a) estimating a confidence interval for each sample, each locus, and each microsatellite length L according to the method of claim 26; and
(b) comparing the estimated confidence intervals;
preferably wherein the two or more samples are from:
(i) different tissues of the same person, preferably a tumor biopsy and a blood sample;
(ii) same tissues sampled at different times, preferably blood, before and after a treatment;
(iii) same tissue, fractionated into components, preferably blood cells and cell-free components of blood; or
(iv) different persons, preferably for forensics, parentage determination, and population studies.

29. (canceled)

30. A method of measuring deviation of microsatellite lengths at one or more loci in a sample relative to microsatellite lengths at the one or more loci of a mono-allelic or bi-allelic baseline sample, the method comprising:
(a) determining a distribution of microsatellite lengths P(l) in the sample at each of the one or more loci according to the method of any one of claims 20 to 22,
(b) calculating a K-eccentricity E_{K}(A,B,P) of the sample to the baseline sample at each of the one or more loci, wherein for a given locus with baseline microsatellite lengths (A, B)
$${E}_{K} ( A , B , P ) = {\sum }_{l} P ( l ) min {( \vert A - l \vert , \vert B - l \vert )}^{K}$$
wherein K is a positive integer and the K-eccentricity E_{K}(A,B,P) is a measure of the deviation of microsatellite lengths at each locus in the sample relative to the microsatellite lengths (A, B) at the locus of a mono-allelic or bi-allelic baseline sample;
preferably wherein:
(1) the mono-allelic or bi-allelic baseline sample is a germline sample;
(ii) the sample:
(1) is a blood sample, preferably blood cells or cell-free component of the blood sample; or
(2) is a sample from a tumor; and/or
(iii) the method further comprises:
(1) calculating the mean K-eccentricity of all mono-C and all d-AC loci of the sample for which a distribution of microsatellite lengths P(l) was determined; and/or
(2) calculating the mean K-eccentricity of all mono-C and all d-AC loci that exhibit low eccentricity in a normal sample.

31. (canceled)

32. A sequencing library:
(a) comprising nucleic acid templates, wherein at least 5%, preferably at least 10%, more preferably at least 208, and more preferably at least 30% of the nucleic acid templates comprise:
(i) a mutagenized microsatellite in which:
(1) at least two, preferably more of the tandem repeat units in the microsatellite have been mutagenized; and
(2) the mutagenized microsatellite has no more than 7, preferably 5 or fewer, tandem repeats in a row; and
(ii) two flanking portions;
preferably wherein the sequencing library:
(1) comprises at least 100, at least 1,000, at least 1×10⁴, at least 3×10⁴, or at least 6×10⁴ nucleic acid templates; and/or
(2) was enriched from a whole genome; or
(b) comprising nucleic acid templates which comprise:
(i) a mutagenized microsatellite in which:
(1) at least two, preferably more of the tandem repeat units in the microsatellite have been mutagenized; and
(2) the mutagenized microsatellite has no more than 7, preferably 5 or fewer, tandem repeats in a row; and
(ii) flanking portions which are not mutagenized;
preferably wherein the sequencing library is a whole genome sequencing library.

33-35. (canceled)

36. A panel comprising a set of 6 or more oligonucleotides with sequences complementary to a flanking portion of microsatellites that are susceptible to the partial mutagenesis of claim 1, preferably wherein:
(a) the microsatellites are at least four repeat units in length;
(b) the microsatellites comprise repeat units, each of which is no more than 10 nucleotides;
(c) the microsatellites are at least 12 nucleotides in length;
(d) the microsatellites are mononucleotide tracts, preferably mono-C tracts;
(e) the microsatellites are dinucleotide tracts, preferably C/G tracts or C/A tracts;
(f) the microsatellites comprise cytosines;
(g the microsatellites comprise adenines;
(h) the microsatellites are known to have repeat length variability;
(i) the microsatellites are more than 5 repeat units in length, more than 7 repeat units in length, more than 10 repeat units in length, more than 15 repeat units in length, more than 20 repeat units in length, more than 30 repeat units in length, between 6 and 70 repeat units in length, between 6 and 32 repeat units in length or between 12 and 64 repeat units in length;
(j) the microsatellite comprises a flanking portion which, together with the microsatellite, maps uniquely to the genome; and/or
(k) the oligonucleotides are complementary to flanking portions that do not hybridize to other flanking portions;
more preferably wherein the panel is:
(i) a panel of hybridization capture probes; or
(ii) a panel of primers to initiate replication.

37-38. (canceled)

39. The panel of claim 36, wherein the sequences of the oligonucleotides are complementary to one or more of the sequences set forth in SEQ ID NOs: 1-1260 and 1891-3150.

40. A kit for performing the method of claim 1.

41. A kit comprising the panel of claim 36, further comprising one or more or all of the following:
(a) synthetic nucleic acid templates, each comprising two flanking portions and a microsatellite of known composition and length, each optionally comprising a varietal tag, a sample barcode, and/or a universal primer binding site, wherein the microsatellite:
(i) comprises nucleotides susceptible to being altered by a step of partial mutagenesis; or
(ii) comprises a known pattern of mutation;
(b) oligonucleotide adaptors, optionally fish-tail adaptors, each of which comprise one or more of the following:
(i) a varietal tag;
(ii) a sample barcode;
(iii) a universal primer binding site; and
(iv) a purification moiety, preferably biotin;
(c) primers to initiate replication, wherein the primers are complementary to:
(i) a universal primer binding site;
(ii) a flanking portion of a synthetic nucleic acid template;
(iii) a flanking portion of an initial nucleic acid template, which initial nucleic acid templates comprise a microsatellite and two flanking portions, optionally wherein the sequences of the primers are complementary to one or more of the sequences set forth in SEQ ID NOs: 1-1260 and 1891-3150; and/or
(iv) a flanking portion of a first copy of an initial nucleic acid template, which initial nucleic acid templates comprise a microsatellite and two flanking portions; and
(d) a set of oligonucleotide blockers complementary to flanking portions of the microsatellites of the panel, optionally wherein the sequences of the oligonucleotide blockers are complementary to one or more of the sequences set forth in SEQ ID NOs: 1-1260 and 1891-3150;
wherein the oligonucleotide adaptors and primers to initiate replication preferably consist of nucleotides which (1) are not susceptible to being altered by a step of partial mutagenesis, and/or (2) are complementary to nucleotides which are not susceptible to being altered by a step of partial mutagenesis;
the kit preferably further comprising:
(i) enzymes or chemicals for partial mutagenesis of initial nucleic acid templates; and/or
(ii) computer-readable media comprising:
(1) an alignment index database comprising, for each locus corresponding to a microsatellite of the panel of the kit, and for each synthetic nucleic acid template of the kit, if present:
(a) a subsequence for each flanking portion and all possible variations that can arise from partial mutagenesis; and
(b) the distance of each said subsequence to the microsatellite; and/or
(2) software for matching sequence reads to subsequences of the alignment index database; and/or
(iii) each of the following:
(1) double-stranded synthetic nucleic acid templates, comprising a flanking portion complementary to a sequence of the panel;
(2) single-stranded synthetic nucleic acid templates, each comprising a microsatellite comprising nucleotides susceptible to being altered by a step of partial mutagenesis; and
(3) single-stranded synthetic nucleic acid templates, each comprising a microsatellite with a known pattern of mutation.

42. (canceled)
